SEQUENCE LISTING (71 sequences)

SEQ ID NO: 1 - DNA, 1359 bp, Artificial Sequence
  PAH
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 2 - DNA, 1359 bp, Artificial Sequence
  Codon optimized PAH (Opt2)
atgagtacggctgtgctcgagaatccaggtttgggccgaaagctgtctgattttggacaggagacatcttatattgaagacaactgcaaccagaatggtgcgatatcccttattttttctctgaaagaagaagtaggtgcgctggcaaaggtcttgcggctgtttgaagagaacgatgttaatcttactcatattgagtccagaccatcacggctgaaaaaagacgagtacgaattttttactcacttggacaaacgaagcttgccggctcttactaatatcattaagatcctccggcatgacataggggcgacagtgcatgagctttcaagggataaaaagaaagataccgtcccctggtttccaaggaccatacaagaactcgaccgattcgcgaaccagatcctttcatatggtgctgagttggatgctgaccaccccggcttcaaagacccggtctaccgagcgcggcggaaacaatttgctgacatcgcatacaattacaggcatggccagccaattcctagagtagaatacatggaagaagagaaaaaaacctggggtaccgtcttcaagacgctgaaatcattgtataaaactcatgcatgttacgaatataaccatatttttccgttgctcgagaaatattgcgggttccacgaagataacatcccacaactcgaggatgtatctcagttcctccagacctgtacggggtttcgacttaggcctgtcgcgggtttgctcagttctcgagacttcctgggtggattggcgtttcgggtattccattgcacgcagtatatccgacacggaagtaagccaatgtacacgccagaacccgatatctgtcacgaattgcttggacacgttcctctgttttctgatcgatcattcgctcagttttcacaggaaatcggcctggcatctttgggagcgccggatgaatatattgagaagctcgctacaatttactggttcacggtagaatttgggttgtgcaagcagggtgatagtattaaagcatacggtgcgggattgctgtcctcattcggggagcttcagtattgcctgtccgagaaacccaagctgttgccgttggaattggaaaaaaccgctatccaaaattacacagtaacggagttccaacctttgtactacgtagccgagtcatttaacgatgcaaaggagaaggtcagaaattttgctgcgacgatacccagaccgttctcagtaaggtacgatccttacactcagaggattgaagtcctggataatacgcaacagctcaagatcctggcagactccataaattctgaaatcggcatcttgtgttcagcactgcaaaagataaaataa

SEQ ID NO: 3 - DNA, 897 bp, Artificial Sequence
  PAH 3′UTR sequence (897 nucleotides)
agccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagattaattaaaatttgggacctgcttcattcaagcttcatatatgctttgcagagaactcataaaggagcatataaggctaaatgtaaaacccaagactgtcattagaattgaattattgggcttaatataaatcgtaacctatgaagtttattttttattttagttaactatgattccaattactactttgttattgtacctaagtaaattttctttaagtcagaagcccattaaaatagttacaagcattgaacttctttagtattatattaatataaaaacatttttgtatgttttattgtaatcataaatactgctgtataaggtaataaaactctgcacctaatccccataacttccagtatcattttccaattaattatcaagtctgttttgggaaacactttgaggacatttatgatgcagcagatgttgactaaaggcttggttggtagatattcaggaaatgttcactgaataaataagtaaatacattattgaaaagcaaatctgtataaatgtgaaatttttatttgtattagtaataaaacattagtagtttaaacaaaaaaaaaaaaaaaaaaaaaaaaaaaaaaactcgactctagatt

SEQ ID NO: 4 - DNA, 289 bp, Artificial Sequence
  PAH 3′UTR sequence (289 nucleotides)
agccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagatta

SEQ ID NO: 5 - DNA, 120 bp, Artificial Sequence
  Prothrombin enhancer (Pro)
gcgagaacttgtgcctccccgtgttcctgctctttgtccctctgtcctacttagactaatatttgccttgggtactgcaaacaggaaatgggggagggacaggagtagggcggagggtag

SEQ ID NO: 6 - DNA, 397 bp, Artificial Sequence
  Human alpha-1 antitrypsin promoter (hAAT)
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 7 - DNA, 573 bp, Artificial Sequence
  Rabbit beta globin intron
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 8 - DNA, 544 bp, Artificial Sequence
  Human beta globin intron
ggatcctgagaacttcagggtgagtctatgggacgcttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 9 - DNA, 13 bp, Artificial Sequence
  1X Hepatocyte Nuclear Factor 1 (1XHNF1)
gttaatcattaac

SEQ ID NO: 10 - DNA, 65 bp, Artificial Sequence
  5X Hepatocyte Nuclear Factor 1 (5XHNF1)
gttaatcattaacgttaatcattaacgttaatcattaacgttaatcattaacgttaatcattaac

SEQ ID NO: 11 - DNA, 30 bp, Artificial Sequence
  1X Hepatocyte Nuclear Factor 1/4 (1XHNF1/4)
gttaatcattaacgcttgtactttggtaca

SEQ ID NO: 12 - DNA, 90 bp, Artificial sequence
  3X Hepatocyte Nuclear Factor 1/4 (3XHNF1/4)
gttaatcattaacgcttgtactttggtacagttaatcattaacgcttgtactttggtacagttaatcattaacgcttgtactttggtaca

SEQ ID NO: 13 - DNA, 53 bp, Artificial sequence
  PAH shRNA sequence #1
tcgcatttcatcaagattaatctcgagattaatcttgatgaaatgcgattttt

SEQ ID NO: 14 - DNA, 53 bp, Artificial sequence
  PAH shRNA sequence #2
actcataaaggagcatataagctcgagcttatatgctcctttatgagtttttt

SEQ ID NO: 15 - DNA, 228 bp, Artificial sequence
  Rous Sarcoma virus (RSV) promoter
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 16 - DNA, 180 bp, Artificial sequence
  5′ Long terminal repeat (LTR)
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 17 - DNA, 41 bp, Artificial sequence
  Psi Packaging signal
tacgccaaaaattttgactagcggaggctagaaggagagag

SEQ ID NO: 18 - DNA, 233 bp, Artificial sequence
  Rev response element (RRE)
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 19 - DNA, 118 bp, Artificial sequence
  Central polypurine tract (cPPT)
ttttaaaagaaaaggggggattggggggtacagtgcaggggaaagaatagtagacataatagcaacagacatacaaactaaagaattacaaaaacaaattacaaaattcaaaatttta

SEQ ID NO: 20 - DNA, 590 bp, Artificial sequence
  Long WPRE sequence
aatcaacctctgattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 21 - DNA, 250 bp, Artificial sequence
  delta U3 3′LTR
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 22 - DNA, 217 bp, Artificial sequence
  H1 Promoter
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactt

SEQ ID NO: 23 - DNA, 642 bp, Artificial sequence
  CMV enhancer/chicken beta actin promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 24 - DNA, 1503 bp, Artificial sequence
  HIV Gag
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 25 - DNA, 1872 bp, Artificial sequence
  HIV Pol
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 26 - DNA, 867 bp, Artificial sequence
  HIV Int
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 27 - DNA, 234 bp, Artificial sequence
  HIV RRE
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 28 - DNA, 351 bp, Artificial sequence
  HIV Rev
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 29 - DNA, 615 bp, Artificial sequence
  CMV Promoter
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactg

SEQ ID NO: 30 - DNA, 1536 bp, Artificial sequence
  VSV-G Envelope Glycoprotein
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtga

SEQ ID NO: 31 - DNA, 141 bp, Artificial sequence
  Left ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 32 - DNA, 228 bp, Artificial sequence
  PolyA
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 33 - DNA, 141 bp, Artificial sequence
  Right ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 1470 bp, Artificial sequence
  E2A
ttaaaagtcgaaggggttctcgcgctcgtcgttgtgcgccgcgctggggagggccacgttgcggaactggtacttgggctgccacttgaactcggggatcaccagtttgggcactggggtctcggggaaggtctcgctccacatgcgccggctcatctgcagggcgcccagcatgtcaggcgcggagatcttgaaatcgcagttggggccggtgctctgcgcgcgcgagttgcggtacactgggttgcagcactggaacaccatcagactggggtacttcacactagccagcacgctcttgtcgctgatctgatccttgtccaggtcctcggcgttgctcaggccgaacggggtcatcttgcacagctggcggcccaggaagggcacgctctgaggcttgtggttacactcgcagtgcacgggcatcagcatcatccccgcgccgcgctgcatattcgggtagagggccttgacgaaggccgcgatctgcttgaaagcttgctgggccttggccccctcgctgaaaaacaggccgcagctcttcccgctgaactgattattcccgcacccggcatcatggacgcagcagcgcgcgtcatggctggtcagttgcaccacgctccgtccccagcggttctgggtcaccttggccttgctgggttgctccttcagcgcacgctgcccgttctcactggtcacatccatctccaccacgtggtccttgtggatcatcaccgtcccatgcagacacttgagctggccttccacctcggtgcagccgtggtcccacagggcactgccggtgcactcccagttcttgtgcgcgatcccgctgtggctgaagatgtaaccttgcaacaggcgacccatgatggtgctaaagctcttctgggtggtgaaggtcagttgcagaccgcgggcctcctcgttcatccaggtctggcacatcttttggaagatctcggtctgctcgggcatgagcttgtaagcatcgcgcaggccgctgtcgacgcggtagcgttccatcagcacattcatggtatccatgcccttctcccaggacgagaccagaggcagactcagggggttgcgcacgttcaggacaccgggggtcgcgggctcgacgatgcgttttccgtccttgccttccttcaacagaaccggcggctggctgaatcccactcccacgatcacggcttcttcctggggcatctcttcgtctgggtctaccttggtcacatgcttggtctttctggcttgcttcttttttggagggctgtccacggggaccacgtcctcctcggaagacccggatcccacccgctgatactttcggcgcttggttggcagaggaggtggcggcgaggggctcctctcctgctccggcggatagcgcgctgaaccgtggccccggggcggagtggcctctcggtccatgaaccggcgcacgtcctgactgccgccggccat

SEQ ID NO: 35 - DNA, 393 bp, Artificial sequence
  E4
tcatgtatctttattgatttttacaccagcacgggtagtcagtctcccaccaccagcccatttcacagtgtaaacaattctctcagcacgggtggccttaaatagggcaatattctgattagtgcgggaactggacttggggtctataatccacacagtttcctggcgagccaaacgggggtcggtgattgagatgaagccgtcctctgaaaagtcatccaagcgagcctcacagtccaaggtcacagtattatgataatctgcatgatcacaatcgggcaacaggggatgttgttcagtcagtgaagccctggtttcctcatcagatcgtggtaaacgggccctgcgatatggatgatggcggagcgagctggattgaatctcggtttgcat

SEQ ID NO: 36 - DNA, 162 bp, Artificial sequence
  VA RNA
agcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttt

SEQ ID NO: 37 - DNA, 2208 bp, Artificial sequence
  AAV2 Rep
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 38 - DNA, 1866 bp, Artificial sequence
  AAV2 Cap
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 39 - DNA, 1608 bp, Artificial sequence
  AAV8 Cap
atggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 40 - DNA, 2214 bp, Artificial sequence
  AAV DJ Cap
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 41 - DNA, 960 bp, Artificial sequence
  Chicken beta actin intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 42 - DNA, 448 bp, Artificial sequence
  Rabbit beta globin poly A
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 43 - DNA, 31 bp, Artificial sequence
  Primer
taagcagaattcatgaatttgccaggaagat

SEQ ID NO: 44 - DNA, 36 bp, Artificial sequence
  Primer
ccatacaatgaatggacactaggcggccgcacgaat

SEQ ID NO: 45 - DNA, 2745 bp, Artificial sequence
  Gag, Pol, Integrase fragment
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 46 - DNA, 1586 bp, Artificial Sequence
  DNA Fragment
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 47 - DNA, 1614 bp, Artificial sequence
  DNA fragment
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 48 - DNA, 884 bp, Artificial sequence
  RSV promoter and HIV Rev
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 49 - DNA, 1104 bp, Artificial sequence
  Elongation Factor-1 alpha (EF1-alpha) promoter
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 50 - DNA, 511 bp, Artificial sequence
  Promoter; PGK
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 51 - DNA, 1162 bp, Artificial sequence
  Promoter; UbC
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 52 - DNA, 120 bp, Artificial sequence
  Poly A; SV40
gtttattgcagcttataatggttacaaataaagcaatagcatcacaaatttcacaaataaagcatttttttcactgcattctagttgtggtttgtccaaactcatcaatgtatcttatca

SEQ ID NO: 53 - DNA, 227 bp, Artificial sequence
  Poly A; bGH
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 54 - DNA, 1695 bp, Artificial sequence
  Envelope; RD114
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 55 - DNA, 2013 bp, Artificial sequence
  Envelope; GALV
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 56 - DNA, 1530 bp, Artificial sequence
  Envelope; FUG
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 57 - DNA, 1497 bp, Artificial sequence
  Envelope; LCMV
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 58 - DNA, 1692 bp, Artificial sequence
  Envelope; FPV
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 59 - DNA, 1266 bp, Artificial sequence
  Envelope; RRV
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 60 - DNA, 1938 bp, Artificial sequence
  Envelope; MLV 10A1
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 61 - DNA, 2030 bp, Artificial sequence
  Envelope; Ebola
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 62 - DNA, 218 bp, Artificial sequence
  Thyroxin binding globulin promoter (TBG)
ctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgag

SEQ ID NO: 63 - DNA, 523 bp, Artificial sequence
  DNA fragment containing prothrombin enhancer and human alpha-1 anti-trypsin promoter
gcgagaacttgtgcctccccgtgttcctgctctttgtccctctgtcctacttagactaatatttgccttgggtactgcaaacaggaaatgggggagggacaggagtagggcggagggtagcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 64 - DNA, 558 bp, Artificial sequence
  DNA fragment containing prothrombin enhancer, human alpha-1 anti-trypsin promoter, and five HNF1 binding sites
gttaatcattaacgttaatcattaacgttaatcattaacgttaatcattaacgttaatcattaacatcgatgcgagaacttgtgcctccccgtgttcctgctctttgtccctctgtcctacttagactaatatttgccttgggtactgcaaacaggaaatgggggagggacaggagtagggcggagggtaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 65 - DNA, 589 bp, Artificial sequence
  DNA fragment containing prothrombin enhancer, human alpha-1, anti-trypsin promoter, and three HNF1/HNF4 binding sites
gttaatcattaacgcttgtactttggtacagttaatcattaacgcttgtactttggtacagttaatcattaacgcttgtactttggtacaatcgatgcgagaacttgtgcctccccgtgttcctgctctttgtccctctgtcctacttagactaatatttgccttgggtactgcaaacaggaaatgggggagggacaggagtagggcggagggtagcccggggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 66 - DNA, 24 bp, Artificial sequence
  Taqman Probe
tcgtgaaagctcatggacagtggc

SEQ ID NO: 67 - DNA, 22 bp, Artificial sequence
  Taqman Forward Primer
agatcttgaggcatgacattgg

SEQ ID NO: 68 - DNA, 22 bp, Artificial sequence
  Taqman Reverse Primer
gtccagctcttgaatggttctt

SEQ ID NO: 69 - DNA, 20 bp, Artificial sequence
  Actin Probe
agcgggaaatcgtgcgtgac

SEQ ID NO: 70 - DNA, 21 bp, Artificial sequence
  Actin Forward Primer
ggacctgactgactacctcat

SEQ ID NO: 71 - DNA, 22 bp, Artificial sequence
  Actin Reverse Primer
cgtagcacagcttctccttaat

(19) United States
(12) Patent Application Publication
(10) Pub. No.: US 2020/0318081 A1
(43) Pub. Date: Oct. 8, 2020

(54) VECTORS WITH PROMOTER AND ENHANCER COMBINATIONS FOR TREATING PHENYLKETONURIA

(71) Applicant: American Gene Technologies International Inc., Rockville, MD (US)

(72) Inventors: Tyler Lahusen, Rockville, MD (US); Charles David Pauza, Rockville, MD (US)

(21) Appl. No.: 16/652,867

(22) PCT Filed: Oct. 2, 2018
(86) PCT No.: PCT/US2018/053919

§ 371 Date: Apr. 1, 2020

Related U.S. Application Data
(60) Provisional application No. 62/566,979, filed on Oct. 2, 2017.

(51) Int. Cl. C12N 7/00; C12N 9/02; C07K 14/805; C07K 14/435; C12N 15/11; C12N 15/86; A61K 48/00; C07K 14/005; C07K 14/81; A61P 43/00

(52) U.S. Cl. CPC C12N 7/00; C12N 2740/15031; C12Y 114/16001; C07K 14/805; C07K 14/435; C12N 15/11; C12N 15/86; A61K 48/0058; C07K 14/005; C07K 14/8125; A61P 43/00; C12N 2830/008; C12N 2740/15043; C12N 2740/15042; C12N 2310/531; C12N 9/0071

(57) ABSTRACT

A lentiviral vector system for expressing a lentiviral particle is disclosed. The lentiviral vector system includes a therapeutic vector. The lentiviral vector system produces a lentiviral particle for upregulating PAH expression in the cells of a subject afflicted with phenylketonuria (PKU).

### CROSS-REFERENCE TO RELATED APPLICATION

This application claims priority to U.S. Provisional Patent Application No. 62/566,979 filed on Oct. 2, 2017, and entitled “VECTORS WITH PROMOTER AND ENHANCER COMBINATIONS FOR TREATING PHENYLKETONURIA”, the disclosure of which is incorporated herein by reference in its entirety.

### FIELD

Aspects of the disclosure relate to genetic medicines for treating phenylketonuria (PKU). More specifically, aspects of the disclosure relate to lentiviral vectors, including PAH-containing lentiviral vectors, whose expression is controlled by various promoter and enhancer combinations.

### BACKGROUND

Phenylketonuria (PKU) refers to a heterogeneous group of disorders that can lead to intellectual disability, seizures, behavioral problems, and impaired growth and development in affected children if left untreated. The mechanisms by which hyperphenylalaninemia results in intellectual impairment reflect the surprising toxicity of high dose phenylalanine and involve hypomyelination or demyelination of nervous system tissues. PKU has an average reported incidence rate of 1 in 12,000 in North America, affecting males and females equally. The disorder is most common in people of European or Native American Ancestry and reaches much higher levels in the eastern Mediterranean region.

Neurological changes in patients with PKU have been demonstrated within one month of birth, and magnetic resonance imaging (MRI) in adult PKU patients has shown white matter lesions in the brain. The size and number these lesions relate directly to blood phenylalanine concentration. The cognitive profile of adolescents and adults with PKU compared with control subjects can include significantly reduced IQ, processing speed, motor control and inhibitory abilities, and reduced performance on tests of attention.

The majority of PKU is caused by a deficiency of hepatic phenylalanine hydroxylase (PAH). PAH is a multimeric hepatic enzyme that catalyzes the hydroxylation of phenylalanine (Phe) to tyrosine (Tyr) in the presence of molecular oxygen and catalytic amounts of tetrahydrobiopterin (BH₄), its nonprotein cofactor. In the absence of sufficient expression of PAH, phenylalanine levels in the blood increase leading to hyperphenylalaninemia and harmful side effects in PKU patients. Decreased or absent PAH activity can lead to a deficiency of tyrosine and its downstream products, including melanin, 1-thyroxine and the catecholamine neurotransmitters including dopamine.

PKU can be caused by mutations in PAH and/or a defect in the synthesis or regeneration of PAH cofactors (i.e., BH₄). Notably, several PAH mutations have been shown to affect protein folding in the endoplasmic reticulum resulting in accelerated degradation and/or aggregation due to missense mutations (63%) and small deletions (13%) in protein structure that attenuate or largely abolish enzyme catalytic activity.

In general, three major phenotypic groups are used to classify PKU based on blood plasma Phe levels, dietary tolerance to Phe and potential responsiveness to therapy. These groups include classical PKU (Phe>1200 μM) atypical or mild PKU (Phe is 600-1200 μM) and permanent mild hyperphenylalaninemia (HPA, Phe 120-600 μM).

Detection of PKU relies on universal newborn screening (NBS). A drop of blood collected from a heel stick is tested for phenylalanine levels in a screen that is mandatory in all 50 states of the USA.

Currently, lifelong dietary restriction of Phe and BH₄ supplementation are the only two available treatment options for PKU, where early therapeutic intervention is critical to ensure optimal clinical outcomes in affected infants. However, costly medication and special low-protein foods imposes a major burden on patients that can lead to malnutrition, psychosocial or neurocognitive complications notably when these products are not fully covered by private health insurance. Moreover, BH₄ therapy is primarily effective for treatment of mild hyperphenylalaninemia as related to defects in BH₄ biosynthesis, whereas only 20-30% of patients with mild or classical PKU are responsive. Thus, there is an urgent need for new treatment modalities for PKU as an alternative to burdensome Phe-restriction diets. Thus, it would be desirable to develop an alternative method for the treatment of phenylketonuria.

Genetic medicines have the potential to effectively treat PKU. Genetic medicines may involve delivery and expression of genetic constructs for the purposes of disease therapy or prevention. Expression of genetic constructs may be modulated by various promoters, enhancers, and/or combinations thereof.

### SUMMARY

In an aspect of the disclosure, a viral vector comprises a therapeutic cargo portion, wherein the therapeutic cargo portion comprises a PAH sequence or a variant thereof, a promoter, and a liver-specific enhancer, wherein the PAH sequence or the variant thereof is operatively controlled by both the promoter and the liver-specific enhancer.

In embodiments, the liver-specific enhancer comprises a prothrombin enhancer. In embodiments, the promoter is a liver-specific promoter. In embodiments, the liver-specific promoter comprises a hAAT promoter. In embodiments, the therapeutic cargo portion further comprises a beta globin intron. In embodiments, the therapeutic cargo portion further comprises at least one hepatocyte nuclear factor binding site. In embodiments, the at least one hepatocyte nuclear factor binding site is disposed upstream of the prothrombin enhancer. In embodiments, the at least one hepatocyte nuclear factor binding site is disposed downstream of the prothrombin enhancer.

In embodiments, the PAH sequence or the variant thereof is truncated. In embodiments, the portion of the PAH sequence or the variant thereof is truncated is the 3′ untranslated region (UTR) of the PAH sequence or the variant thereof.

In embodiments, the PAH sequence or the variant thereof comprises a sequence having at least 80%, or at least 85%, or at least 90%, or at least 95% percent identity with:

(SEQ ID NO: 1)
ATGTCCACTGCGGTCCTGGAAAACCCAGGCTTGGGCAGGAAACTCT
CTGACTTTGGACAGGAAACAAGCTATATTGAAGACAACTGCAATCA
AAATGGTGCCATATCACTGATCTTCTCACTCAAAGAAGAAGTTGGT
GCATTGGCCAAAGTATTGCGCTTATTTGAGGAGAATGATGTAAACC
TGACCCACATTGAATCTAGACCTTCTCGTTTAAAGAAAGATGAGTA
TGAATTTTTCACCCATTTGGATAAACGTAGCCTGCCTGCTCTGACAA
ACATCATCAAGATCTTGAGGCATGACATTGGTGCCACTGTCCATGA
GCTTTCACGAGATAAGAAGAAAGACACAGTGCCCTGGTTCCCAAG
AACCATTCAAGAGCTGGACAGATTTGCCAATCAGATTCTCAGCTAT
GGAGCGGAACTGGATGCTGACCACCCTGGTTTTAAAGATCCTGTGT
ACCGTGCAAGACGGAAGCAGTTTGCTGACATTGCCTACAACTACCG
CCATGGGCAGCCCATCCCTCGAGTGGAATACATGGAGGAAGAAAA
GAAAACATGGGGCACAGTGTTCAAGACTCTGAAGTCCTTGTATAAA
ACCCATGCTTGCTATGAGTACAATCACATTTTTCCACTTCTTGAAAA
GTACTGTGGCTTCCATGAAGATAACATTCCCCAGCTGGAAGACGTT
TCTCAATTCCTGCAGACTTGCACTGGTTTCCGCCTCCGACCTGTGGC
TGGCCTGCTTTCCTCTCGGGATTTCTTGGGTGGCCTGGCCTTCCGAG
TCTTCCACTGCACACAGTACATCAGACATGGATCCAAGCCCATGTA
TACCCCCGAACCTGACATCTGCCATGAGCTGTTGGGACATGTGCCC
TTGTTTTCAGATCGCAGCTTTGCCCAGTTTTCCCAGGAAATTGGCCT
TGCCTCTCTGGGTGCACCTGATGAATACATTGAAAAGCTCGCCACA
ATTTACTGGTTTACTGTGGAGTTTGGGCTCTGCAAACAAGGAGACT
CCATAAAGGCATATGGTGCTGGGCTCCTGTCATCCTTTGGTGAATT
ACAGTACTGCTTATCAGAGAAGCCAAAGCTTCTCCCCCTGGAGCTG
GAGAAGACAGCCATCCAAAATTACACTGTCACGGAGTTCCAGCCCC
TGTATTACGTGGCAGAGAGTTTTAATGATGCCAAGGAGAAAGTAA
GGAACTTTGCTGCCACAATACCTCGGCCCTTCTCAGTTCGCTACGA
CCCATACACCCAAAGGATTGAGGTCTTGGACAATACCCAGCAGCTT
AAGATTTTGGCTGATTCCATTAACAGTGAAATTGGAATCCTTTGCA
GTGCCCTCCAGAAAATAAAGTAA;
(SEQ ID NO: 2)
ATGAGTACGGCTGTGCTCGAGAATCCAGGTTTGGGCCGAAAGCTGT
CTGATTTTGGACAGGAGACATCTTATATTGAAGACAACTGCAACCA
GAATGGTGCGATATCCCTTATTTTTTCTCTGAAAGAAGAAGTAGGT
GCGCTGGCAAAGGTCTTGCGGCTGTTTGAAGAGAACGATGTTAATC
TTACTCATATTGAGTCCAGACCATCACGGCTGAAAAAAGACGAGTA
CGAATTTTTTACTCACTTGGACAAACGAAGCTTGCCGGCTCTTACTA
ATATCATTAAGATCCTCCGGCATGACATAGGGGCGACAGTGCATGA
GCTTTCAAGGGATAAAAAGAAAGATACCGTCCCCTGGTTTCCAAGG
ACCATACAAGAACTCGACCGATTCGCGAACCAGATCCTTTCATATG
GTGCTGAGTTGGATGCTGACCACCCCGGCTTCAAAGACCCGGTCTA
CCGAGCGCGGCGGAAACAATTTGCTGACATCGCATACAATTACAG
GCATGGCCAGCCAATTCCTAGAGTAGAATACATGGAAGAAGAGAA
AAAAACCTGGGGTACCGTCTTCAAGACGCTGAAATCATTGTATAAA
ACTCATGCATGTTACGAATATAACCATATTTTTCCGTTGCTCGAGAA
ATATTGCGGGTTCCACGAAGATAACATCCCACAACTCGAGGATGTA
TCTCAGTTCCTCCAGACCTGTACGGGGTTTCGACTTAGGCCTGTCGC
GGGTTTGCTCAGTTCTCGAGACTTCCTGGGTGGATTGGCGTTTCGG
GTATTCCATTGCACGCAGTATATCCGACACGGAAGTAAGCCAATGT
ACACGCCAGAACCCGATATCTGTCACGAATTGCTTGGACACGTTCC
TCTGTTTTCTGATCGATCATTCGCTCAGTTTTCACAGGAAATCGGCC
TGGCATCTTTGGGAGCGCCGGATGAATATATTGAGAAGCTCGCTAC
AATTTACTGGTTCACGGTAGAATTTGGGTTGTGCAAGCAGGGTGAT
AGTATTAAAGCATACGGTGCGGGATTGCTGTCCTCATTCGGGGAGC
TTCAGTATTGCCTGTCCGAGAAACCCAAGCTGTTGCCGTTGGAATT
GGAAAAAACCGCTATCCAAAATTACACAGTAACGGAGTTCCAACC
TTTGTACTACGTAGCCGAGTCATTTAACGATGCAAAGGAGAAGGTC
AGAAATTTTGCTGCGACGATACCCAGACCGTTCTCAGTAAGGTACG
ATCCTTACACTCAGAGGATTGAAGTCCTGGATAATACGCAACAGCT
CAAGATCCTGGCAGACTCCATAAATTCTGAAATCGGCATCTTGTGT
TCAGCACTGCAAAAGATAAAATAA;
(SEQ ID NO: 3)
AGCCATGGACAGAATGTGGTCTGTCAGCTGTGAATCTGTTGATGGA
GATCCAACTATTTCTTTCATCAGAAAAAGTCCGAAAAGCAAACCTT
AATTTGAAATAACAGCCTTAAATCCTTTACAAGATGGAGAAACAAC
AAATAAGTCAAAATAATCTGAAATGACAGGATATGAGTACATACT
CAAGAGCATAATGGTAAATCTTTTGGGGTCATCTTTGATTTAGAGA
TGATAATCCCATACTCTCAATTGAGTTAAATCAGTAATCTGTCGCAT
TTCATCAAGATTAATTAAAATTTGGGACCTGCTTCATTCAAGCTTCA
TATATGCTTTGCAGAGAACTCATAAAGGAGCATATAAGGCTAAATG
TAAAACCCAAGACTGTCATTAGAATTGAATTATTGGGCTTAATATA
AATCGTAACCTATGAAGTTTATTTTTTATTTTAGTTAACTATGATTC
CAATTACTACTTTGTTATTGTACCTAAGTAAATTTTCTTTAAGTCAG
AAGCCCATTAAAATAGTTACAAGCATTGAACTTCTTTAGTATTATA
TTAATATAAAAACATTTTTGTATGTTTTATTGTAATCATAAATACTG
CTGTATAAGGTAATAAAACTCTGCACCTAATCCCCATAACTTCCAG
TATCATTTTCCAATTAATTATCAAGTCTGTTTTGGGAAACACTTTGA
GGACATTTATGATGCAGCAGATGTTGACTAAAGGCTTGGTTGGTAG
ATATTCAGGAAATGTTCACTGAATAAATAAGTAAATACATTATTGA
AAAGCAAATCTGTATAAATGTGAAATTTTTATTTGTATTAGTAATA
AAACATTAGTAGTTTAAACAAAAAAAAAAAAAAAAAAAAAAAAA
AAAAAACTCGACTCTAGATT;
or
(SEQ ID NO: 4)
AGCCATGGACAGAATGTGGTCTGTCAGCTGTGAATCTGTTGATGGA
GATCCAACTATTTCTTTCATCAGAAAAAGTCCGAAAAGCAAACCTT
AATTTGAAATAACAGCCTTAAATCCTTTACAAGATGGAGAAACAAC
AAATAAGTCAAAATAATCTGAAATGACAGGATATGAGTACATACT
CAAGAGCATAATGGTAAATCTTTTGGGGTCATCTTTGATTTAGAGA
TGATAATCCCATACTCTCAATTGAGTTAAATCAGTAATCTGTCGCAT
TTCATCAAGATTA.

In embodiments, the PAH sequence or the variant thereof comprises: SEQ ID NO: 1; SEQ ID NO: 2; SEQ ID NO: 3; or SEQ ID NO: 4.

In embodiments, the prothrombin enhancer comprises a sequence having at least 80%, or at least 85%, or at least 90%, or at least 95% percent identity with:

(SEQ ID NO: 5)
GCGAGAACTTGTGCCTCCCCGTGTTCCTGCTCTTTGTCCCTCTGTCC
TACTTAGACTAATATTTGCCTTGGGTACTGCAAACAGGAAATGGGG
GAGGGACAGGAGTAGGGCGGAGGGTAG.

In embodiments, the sequence of prothrombin enhancer comprises SEQ ID NO: 5.

In embodiments, the sequence of the hAAT promoter comprises SEQ ID NO: 6. In embodiments, the sequence of the beta globin intron comprises one of SEQ ID NOs: 7 or 8. In embodiments, the sequence of the hepatocyte nuclear factor binding site comprises any one of SEQ ID NOs: 9-12.

In embodiments, the therapeutic cargo portion further comprises at least one small RNA sequence that is capable of binding to at least one pre-determined complementary mRNA sequence. In embodiments, the at least one small RNA sequence targets a complementary mRNA sequence that contains a full-length UTR. In embodiments, the at least one pre-determined complementary mRNA sequence is a PAH mRNA sequence. In embodiments, the at least one small RNA sequence inhibits production of endogenous PAR. In embodiments, the at least one small RNA sequence comprises a shRNA. In embodiments, the at least one small RNA sequence is under the control of a first promoter and the PAH sequence or the variant thereof is under the control of a second promoter. In embodiments, the first promoter comprises a H1 promoter. In embodiments, the second promoter comprises a liver-specific promoter. In embodiments, the liver-specific promoter comprises a hAAT promoter. In embodiments, the at least one small RNA sequence comprises a sequence having at least 80%, or at least 85%, or at least 90%, or at least 95% percent identity with:

(SEQ ID NO: 13)
TCGCATTTCATCAAGATTAATCTCGAGATTAATCTTGATGAAATGC
GATTTTT;
or
(SEQ ID NO: 14)
ACTCATAAAGGAGCATATAAGCTCGAGCTTATATGCTCCTTTATGA
GTTTTTT.

In embodiments, the at least one small RNA sequence comprises SEQ ID NO: 13; or SEQ ID NO: 14.

In embodiments, the viral vector is a lentiviral vector. In embodiments, the viral vector is an AAV vector.

In an aspect of the disclosure, a lentiviral particle capable of infecting a target cell comprises an envelope protein optimized for infecting the target cell, and the viral vector according to any of the embodiments of the disclosure. In embodiments, the target cell is a hepatic cell, a muscle cell, an epithelial cell, an endothelial cell, a neural cell, a neuroendocrine cell, an endocrine cell, a lymphocyte, a myeloid cell, a cell present within a solid organ, or cell of a hematopoietic lineage, a hematopoietic stem cell, or a precursor hematopoietic stem cell.

In an aspect of the disclosure, a method of treating PKU in a subject is disclosed. The method comprises administering to the subject a therapeutically effective amount of the lentiviral particle described herein. In an aspect of the disclosure, a method of preventing PKU in a subject comprises administering to the subject a therapeutically effective amount of the lentiviral particle described herein. In embodiments, the method further comprises diagnosing a PKU genotype in the subject that correlates with a PKU phenotype. In embodiments, the subject is in utero. In embodiments, the diagnosing occurs during prenatal screening of the subject. In embodiments, the diagnosing occurs in vitro. In embodiments, the therapeutically effective amount of the lentiviral particle comprises a plurality of single doses of the lentiviral particle. In embodiments, the therapeutically effective amount of the lentiviral particle comprises a single dose of the lentiviral particle.

### BRIEF DESCRIPTION OF THE DRAWINGS

FIG. 1 depicts an exemplary 3-vector lentiviral vector system in a circularized form.

FIG. 2 depicts an exemplary 4-vector lentiviral vector system in a circularized form.

FIG. 3 depicts linear maps of eight exemplary lentiviral vectors containing variations of the prothrombin enhancer and hAAT promoter to regulate the expression of PAH.

FIGS. 4A and 4B depict immunoblot data comparing levels of PAH with different enhancer elements and with or without the 3′ UTR in (FIG. 4A) Hepa1-6 and (FIG. 4B) 293T cells.

FIGS. 5A-5C depict immunoblot data comparing levels of PAH in Hepa1-6 cells (FIG. 5A) with or without a rabbit beta globin intron, (FIG. 5B) a codon-optimized PAH sequence, and (FIG. 5C) a prothrombin enhancer with a HNF1 or HNF1/4 binding site either upstream or downstream.

FIG. 6 depicts PAH RNA expression in Hepa1-6 cells transduced with lentiviral vectors expressing PAH via variations in the prothrombin enhancer.

FIG. 7 depicts immunoblot data comparing levels of PAH expression with either the anti-alpha 1 trypsin (hAAT) or thyroxin-binding globulin (TBG) promoter in Hepa1-6 cells.

FIGS. 8A and 8B depict immunoblot data comparing levels of PAH with or without a rabbit or human beta globin intron in Hepa1-6 cells (FIG. 8A) or Hep3B cells (FIG. 8B).

FIG. 9 depicts immunoblot data of PAH expression in human primary hepatocytes with lentiviral vectors expressing PAH.

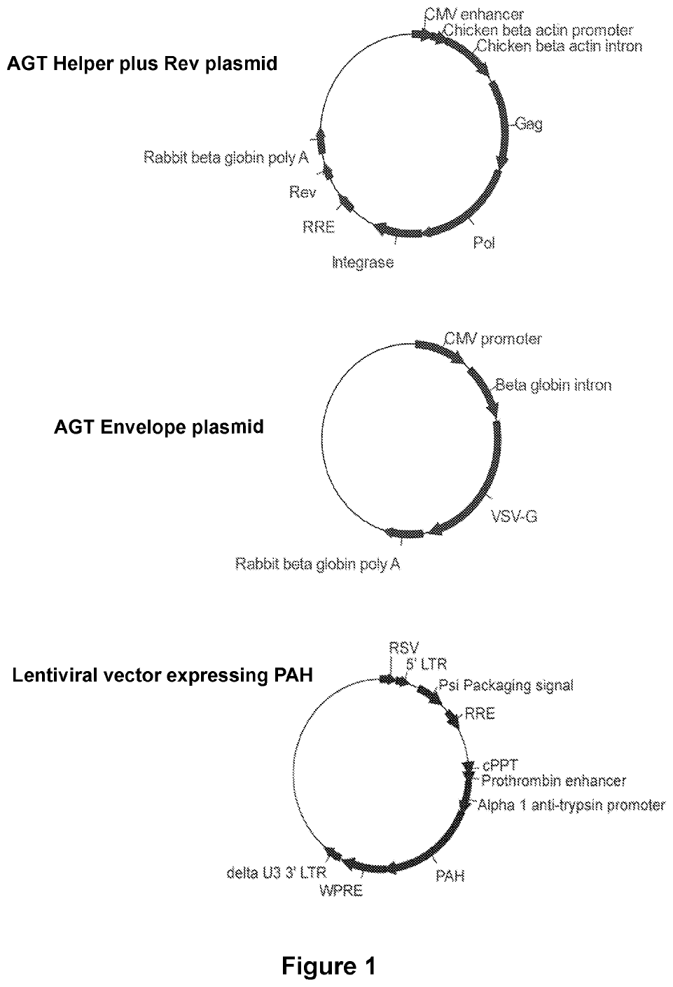

FIGS. 1A-10C depict PAH activity by detection of phenylalanine levels in (FIGS. 10A and 10C) cell media or (FIG. 10B) lysate of Hepa1-6 cells that were transduced with lentiviral vectors expressing PAH and treatment with sepiapterin, a BH4 cofactor precursor.

FIG. 11 depicts decreased levels of Phe in blood of Pah^{enu2} mice after treatment with a lentiviral vector containing PA.

FIG. 12 depicts blood phenylalanine suppression by LV-Pro-hAAT-PAH.

FIGS. 13A-13D depict PAH protein expression (FIG. 13A) and PAH RNA expression (FIG. 13D) after AAV-delivered expression of PAH in 293 cells with various DJ or AAV/2 serotype vectors; fold changes of PAH protein expression were also analyzed after delivery of the AAV/DJ vectors (FIG. 13B) and the AAV/2 vectors (FIG. 13C).

FIG. 14 depicts decreased levels of Phe in neonatal enu2/enu2 mice treated with LV-Pro-hAAT-PAH lentivirus vector therapy for PKU.

FIG. 15 depicts data from Hep3B cells showing PAH expression following treatment with lentiviral vectors encoding prothrombin-hAAT-PAH-PAH shRNA sequence #1 (SEQ ID NO: 13) or prothrombin-hAAT-PAH-PAH shRNA sequence #2 (SEQ ID NO: 14), each of which target the 3′UTR of the mRNA expressed by the endogenous Pah gene and inhibit expression of the PAH protein.

### DETAILED DESCRIPTION

### Overview of the Disclosure

This disclosure relates to therapeutic vectors and delivery of the same to cells. In embodiments, the therapeutic vectors include PAH sequences or variants thereof, and a liver-specific enhancer. In embodiments, the therapeutic vectors also include a small RNA that regulates host (i.e., endogenous) PAH protein expression.

### Definitions and Interpretation

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used in the description and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “administration of” or “administering” an active agent should be understood to mean providing an active agent to the subject in need of treatment in a form that can be introduced into that individual's body in a therapeutically useful form and therapeutically effective amount.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “expression”, “expressed”, or “encodes” refers to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

As used herein, the term “adeno-associated viral vector,” refers to a carrier or transporter of that adeno-associated virus. The term “adeno-associated viral vector” may also be referred to herein as an “AAV vector”.

As used herein, the term “adeno-associated virus,” refers to a small virus that generates a mild immune response, is capable of integrating into a host cell genome, and is not pathogenic.

As used herein, the term “AAV/DJ” (also referred to herein as “AAV-DJ”) is a serotype of an AAV vector engineered from different AAV serotypes, which mediates higher transduction and infectivity rates than wild-type AAV serotypes.

As used herein, the term “AAV2” (also referred to herein as “AAV/2” or “AAV-2”) is a naturally occurring AAV serotype.

As used herein, the term “AAV-Pro-hAAT-PAH” refers to an AAV vector comprising a prothrombin enhancer, a hAAT promoter, and a PAH sequence.

As used herein, the abbreviation “ApoE enhancer” refers to an Apolipoprotein E enhancer.

As used herein, the term “genetic medicine” or “genetic medicines” refers generally to therapeutics and therapeutic strategies that focus on genetic targets to treat a clinical disease or manifestation. The term “genetic medicine” encompasses gene therapy and the like.

As used herein, the abbreviation “hAAT” refers to a hAAT promoter.

As used herein, the term “hAAT-hPAH-3′UTR₂₈₉” may also be referred to herein as U₂₈₉, or generally as transgene-expressed truncated hPAH 3′UTR, or generally a truncated 3′ UTR.

As used herein, the term “hepatocyte nuclear factors” refers to transcription factors that are predominantly expressed in the liver. Types of hepatocyte nuclear factors include, but are not limited to, hepatocyte nuclear factor 1, hepatocyte nuclear factor 2, hepatocyte nuclear factor 3, and hepatocyte nuclear factor 4.

As used herein, the abbreviation “HNF” refers to hepatocyte nuclear factor. Accordingly HNF1 refers to hepatocyte nuclear factor 1, HNF2 refers to hepatocyte nuclear factor 2, HNF3 refers to hepatocyte nuclear factor 3, and HNF4 refers to hepatocyte nuclear factor 4.

As used herein, the phrase “HNF binding site,” refers to a region of DNA to which a HNF transcription factor can bind. Accordingly, a HNF1 binding site is a region of DNA to which HNF1 can bind, and a HNF4 binding site is a region of DNA to which HNF4 can bind.

As used herein, the terms “individual,” “subject,” and “patient” are used interchangeably herein, and refer to any individual mammal subject, e.g., murine, porcine, bovine, canine, feline, equine, nonhuman primate or human primate.

As used herein, the phrase “rabbit beta globin intron” refers to a nucleic acid segment within the rabbit beta globin gene that is spliced out during RNA maturation, and does not code for a protein.

As used herein, the phrase “human beta globin intron” refers to a nucleic acid segment within the human beta globin gene that is spliced out during RNA maturation, and does not code for a protein.

As used herein, the term “LV” refers generally to “lentivirus.” As a non-limiting example, reference to “LV-PAH” is reference to a lentivirus that contains a PAH sequence and expresses PAH.

As used herein, the term “LV-Pro-hAAT-PAH” refers to a lentivirus comprising a prothrombin enhancer, a hAAT promoter, and a PAH sequence. The LV-Pro-hAAT-PAH vector is also referred to as the AGT323 vector.

As used herein, the term “LV-HNF-Pro-hAAT-PAH” refers to a lentivirus comprising a HNF biding site, a prothrombin enhancer, a hAAT promoter, and a PAH sequence.

As used herein, the term “LV-Pro-intron-PAH” refers to a lentivirus comprising a prothrombin enhancer, an intron, and a PAH sequence, wherein the intron is the human beta globin intron.

As used herein, the term “LV-Pro-hAAT” refers to a lentivirus comprising a prothrombin enhancer and a hAAT promoter.

As used herein, the term “LV-Pro-TBG-PAH” refers to a lentivirus comprising a prothrombin enhancer, a thyroxin binding globulin, and a PAH sequence.

As used herein, the term “LV-ApoE-hAAT-PAH-UTR” refers to a lentivirus comprising an apolipoprotein E enhancer, a hAAT promoter, a PAH sequence, and an untranslated region of a gene, wherein the untranslated region is the 3′UTR of the PAH gene.

As used herein, the term “LV-Pro-hAAT-PAH-shPAH” refers to a lentivirus comprising a prothrombin enhancer, a hAAT promoter, a PAH sequence and a shPAH sequence.

As used herein, the term “packaging cell line” refers to any cell line that can be used to express a lentiviral particle.

As used herein, the term “percent identity”, in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

As used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J. Pharm. Sci. 66:1-19).

The term “phenylalanine hydroxylase” may also be referred to herein as PA. The term phenylalanine hydroxylase includes all wild-type and variant PAH sequences, including both nucleotide and peptide sequences. Without limitation, the term phenylalanine hydroxylase includes reference to SEQ ID NOs: 1-4, and further includes variants having at least about 80% identity therewith. Human PAH may also be referred to herein as hPAH. Human PAH may also be referred to herein as hPAH.

As used herein, the term “wild-type hPAH” may also be referred to herein as endogenous PAH or “full-length PAH”.

As used herein, the term “phenylketonuria”, which is also referred to herein as “PKU”, refers to the chronic deficiency of phenylalanine hydroxylase, as well as all symptoms related thereto including mild and classical forms of disease. Treatment of “phenylketonuria”, therefore, may relate to treatment for all or some of the symptoms associated with PKU.

As used herein, the term “prothrombin enhancer” is a region on the prothrombin gene that can be bound by proteins, which results in transcription of the prothrombin gene.

As used herein, the abbreviation “Pro” refers to a prothrombin enhancer.

As used herein, “small RNA” refers to non-coding RNA that are generally about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA). “Small RNA” of the disclosure should be capable of inhibiting or knocking-down gene expression of a target gene, generally through pathways that result in the destruction of the target gene mRNA.

As used herein, the term “shPAH” refers to a small hairpin RNA that targets PAH.

As used herein, the abbreviation “lncRNA” refers to a long non-coding RNA.

As used herein, the term “SEQ ID NO” is synonymous with the term “Sequence ID No.”

As used herein, the term “thyroxin binding globulin,” is a transport protein responsible for carrying thyroid hormones in the bloodstream. As used herein, the abbreviation “TBG” refers to thyroxin binding globulin.

As used herein, the term “therapeutically effective amount” refers to a sufficient quantity of the active agents of the present disclosure, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

As used herein, the term “therapeutic vector” includes, without limitation, reference to a lentiviral vector or an adeno-associated viral (AAV) vector. Additionally, as used herein with reference to the lentiviral vector system, the term “vector” is synonymous with the term “plasmid”. For example, the 3-vector and 4-vector systems, which include the 2-vector and 3-vector packaging systems, can also be referred to as 3-plasmid and 4-plasmid systems.

As used herein, the term “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis.

“A treatment” is intended to target the disease state and combat it, i.e., ameliorate or prevent the disease state. The particular treatment thus will depend on the disease state to be targeted and the current or future state of medicinal therapies and therapeutic approaches. A treatment may have associated toxicities.

As used herein, the term “truncated” may also be referred to herein as “shortened” or “without”.

As used herein, the term “UTR” is in reference to a region of a gene that is either 5′ or 3′ of the coding region of a gene.

As used herein, the term “3′ UTR” is the “UTR” that is 3′ of the coding region of a gene.

As used herein, the term “variant” may also be referred to herein as analog or variation. A variant refers to any substitution, deletion, or addition to a nucleotide sequence.

As considered herein, optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules provided in the disclosure. BLAST protein searches can be performed with the XBLAST program, score=50, word length=3 to obtain amino acid sequences homologous to the protein molecules of the disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

### Description of Aspects and Embodiments of the Disclosure

In an aspect of the disclosure, a viral vector comprises a therapeutic cargo portion, wherein the therapeutic cargo portion comprises a PAH sequence or a variant thereof, a promoter, and a liver-specific enhancer, wherein the PAH sequence or the variant thereof is operatively controlled by both the promoter and the liver-specific enhancer.

In embodiments, the liver-specific enhancer comprises a prothrombin enhancer. In embodiments, the promoter is a liver-specific promoter. In embodiments, the liver-specific promoter comprises a hAAT promoter. In embodiments, the therapeutic cargo portion further comprises a beta globin intron. In embodiments, the therapeutic cargo portion further comprises at least one hepatocyte nuclear factor binding site. In embodiments, the at least one hepatocyte nuclear factor binding site is disposed upstream of the prothrombin enhancer. In embodiments, the at least one hepatocyte nuclear factor binding site is disposed downstream of the prothrombin enhancer.

In embodiments, a lentiviral vector is provided comprising a prothrombin enhancer, a hAAT promoter, and a PAH sequence (LV-Pro-hAAT-PAH). In embodiments, a lentiviral vector is provided comprising a HNF binding site, a prothrombin enhancer, a hAAT promoter, and a PAH sequence (LV-HNF-Pro-hAAT-PAH). In embodiments, the HNF binding site is a HNF1 or HNF/4 binding site. In embodiments, a lentiviral vector is provided comprising a prothrombin enhancer, a hAAT promoter, an intron, and a PAH sequence (LV-Pro-intron-PAH). In embodiments, the intron is a rabbit globin intron. In embodiments, the intron is a human globin intron. In embodiments, a lentiviral vector is provided comprising a prothrombin enhancer and a hAAT promoter (LV-Pro-hAAT). In embodiments, a lentiviral vector is provided comprising a prothrombin enhancer, a thyroxin binding globulin, and a PAH sequence (LV-Pro-TBG-PAH). In embodiments, a lentiviral vector is provided comprising a ApoE enhancer, a hAAT promoter, a PAH sequence, and the 3′UTR of PAH (LV-ApoE-hAAT-PAH-UTR).

In embodiments, the PAH sequence or the variant thereof is truncated. In embodiments, the portion of the PAH sequence or the variant thereof that is truncated is the 3′ untranslated region (UTR) of the PAH sequence or the variant thereof.

In embodiments, the PAH truncation at the 3′UTR prevents binding of certain regulatory RNA to the 3′UTR. In embodiments, the regulatory RNA is a lncRNA. In embodiments, the regulatory RNA is a microRNA. In embodiments, the regulatory RNA is a piRNA. In embodiments, the regulatory RNA is a shRNA. In embodiments the regulatory RNA is a siRNA between 19 and 25 nucleotides in length. In embodiments, the regulatory RNA is a small RNA sequence comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NOs: 13 or 14.

In embodiments, the PAH sequence comprises SEQ ID NO: 1. In embodiments, the PAH sequence comprises a codon optimized PAH sequence (SEQ ID NO: 2). In embodiments, the PAH sequence or the variant thereof comprises a truncated 3′ UTR (289 nucleotides) (SEQ ID NO: 4). In embodiments, the PAH sequence or the variant thereof comprises a 5′ UTR (897 nucleotides) (SEQ ID NO: 3).

In embodiments, the PAH sequence or the variant thereof comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 1; SEQ ID NO: 2; SEQ ID NO: 3; or SEQ ID NO: 4.

In embodiments, variants can be made to any of the above-described sequences. In embodiments, the PAH sequence or the variant thereof comprises SEQ ID NO: 1; SEQ ID NO: 2; SEQ ID NO: 3; or SEQ ID NO: 4.

In embodiments, the prothrombin enhancer comprises a sequence having at least 80%, or at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 5

In embodiments, variants can be made to the above-described sequences. In embodiments, the sequence of prothrombin enhancer comprises SEQ ID NO: 5.

In embodiments, the sequence of the hAAT promoter comprises SEQ ID NO: 6. In embodiments, the sequence of the beta globin intron comprises one of SEQ ID NOs: 7 or 8. In embodiments, the sequence of the hepatocyte nuclear factor binding site comprises any one of SEQ ID NOs: 9-12.

In embodiments, the therapeutic cargo portion further comprises at least one small RNA sequence that is capable of binding to at least one pre-determined complementary mRNA sequence. In embodiments, the at least one small RNA sequence targets a complementary mRNA sequence that contains a full-length UTR. In embodiments, the at least one pre-determined complementary mRNA sequence is a PAH mRNA sequence. In embodiments, the at least one small RNA sequence comprises a shRNA. In embodiments, the at least one small RNA sequence is under the control of a first promoter and the PAH sequence or the variant thereof is under the control of a second promoter. In embodiments, the first promoter comprises a H1 promoter. In embodiments, the second promoter comprises a liver-specific promoter. In embodiments, the liver-specific promoter comprises a hAAT promoter. In embodiments, the at least one small RNA sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NO: 13; or SEQ ID NO: 14.

In embodiments, variants can be made to any of the above-described sequences. In embodiments, the at least one small RNA sequence comprises SEQ ID NO: 13; or SEQ ID NO: 14.

In embodiments, a lentiviral vector is provided comprising a prothrombin enhancer, a hAAT promoter, a PAH sequence, and a shRNA that targets endogenous PAH (LV-Pro-hAAT-PAH-shPAH). In embodiments, the shRNA targets the 3′UTR of endogenous PA. In embodiments, the shPAH comprises SEQ ID NO: 13. In embodiments, the shPAH comprises SEQ ID NO: 14.

In an aspect of the disclosure, a lentiviral particle capable of infecting a target cell comprises an envelope protein optimized for infecting the target cell, and any of the viral vectors disclosed herein. In embodiments, the target cell is a hepatic cell, a muscle cell, an epithelial cell, an endothelial cell, a neural cell, a neuroendocrine cell, an endocrine cell, a lymphocyte, a myeloid cell, a cell present within a solid organ, or cell of a hematopoietic lineage, a hematopoietic stem cell, or a precursor hematopoietic stem cell.

In an aspect of the disclosure, a method of treating PKU in a subject comprises administering to the subject a therapeutically effective amount of any of the lentiviral particles disclosed herein. In an aspect of the disclosure, a method of preventing PKU in a subject comprises administering to the subject a therapeutically effective amount of any of the lentiviral particles disclosed herein. In another aspect of the disclosure, use of a therapeutically effective amount of any of the lentiviral particles disclosed herein for treating PKU in a subject is disclosed. In embodiments, the method further comprises diagnosing a PKU genotype in the subject that correlates with a PKU phenotype. In embodiments, the subject is in utero. In embodiments, the diagnosing occurs during prenatal screening of the subject or after genetic screening of the parents. In embodiments, the diagnosing occurs in vitro. In embodiments, the therapeutically effective amount of the lentiviral particle comprises a plurality of single doses of the lentiviral particle. In embodiments, the therapeutically effective amount of the lentiviral particle comprises a single dose of the lentiviral particle.

In an aspect of the disclosure, a method of treating PKU in a subject is provided which comprises treating a subject that has a mutant form of PAH with a therapeutically effective amount of a lentiviral vector comprising exogenous PA. In embodiments, the subject is a mammal. In embodiments, the mammal is a human. In embodiments, the mammal is a rodent. In embodiments, the rodent is a mouse or a rat. In embodiments, the mammal is porcine.

In embodiments, the subject is treated with a lentiviral vector. In embodiments the lentiviral vector comprises a PAH sequence or a variant thereof. In embodiments, the PAH sequence is any of the PAH sequences or variants described herein.

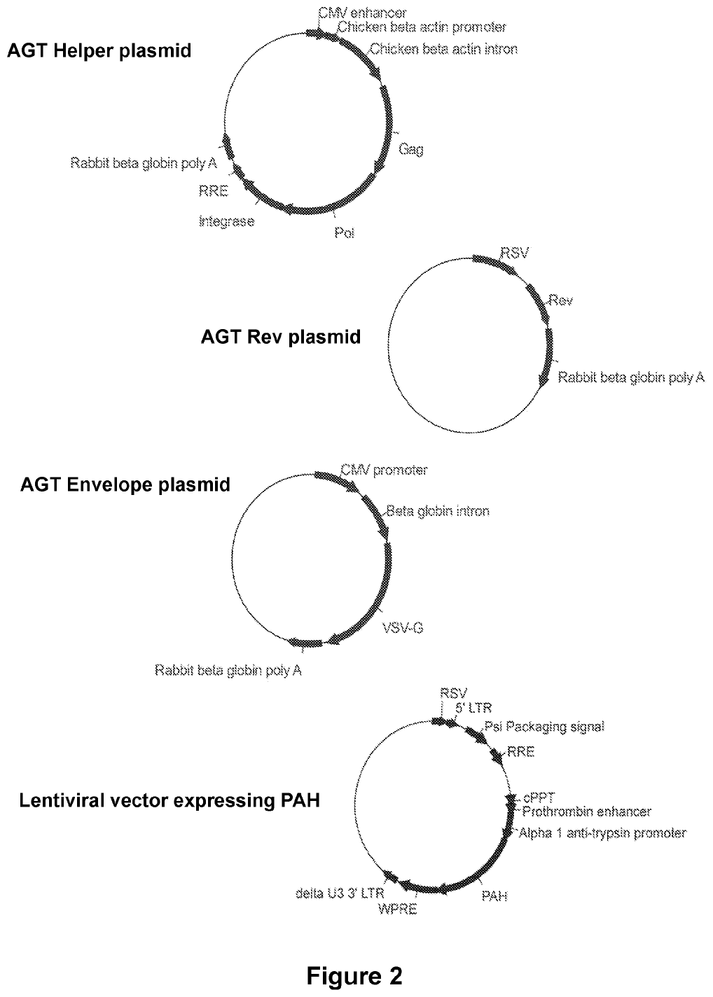

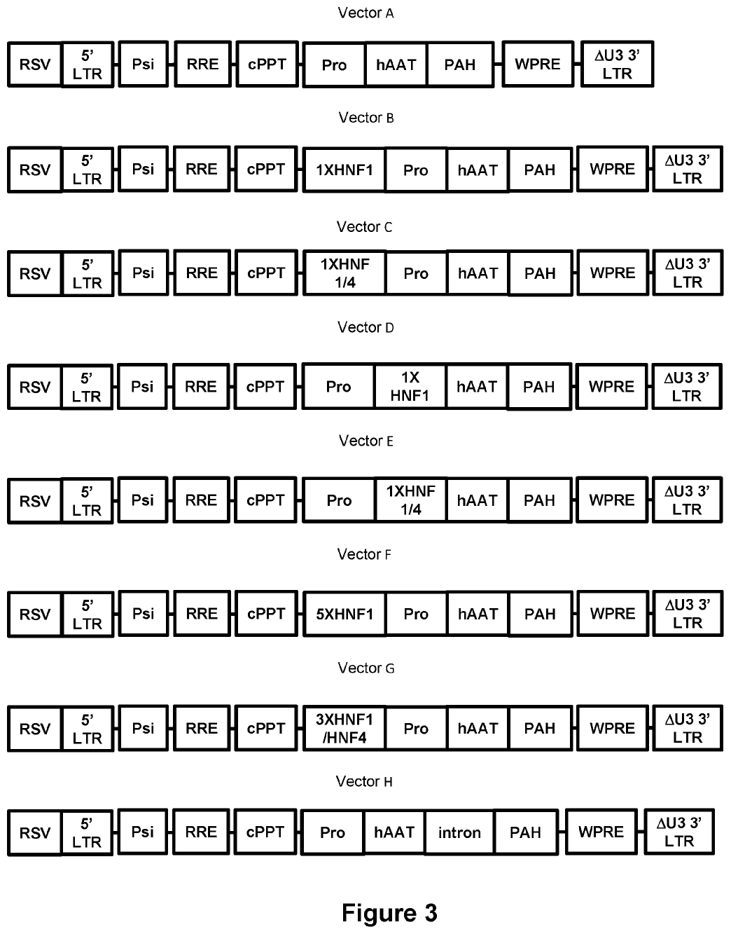

In embodiments, the lentiviral vector is any of the lentiviral vectors comprising PAH or variants described herein. In embodiments, the lentiviral vector comprising PAH is a lentiviral vector expressing PAH as depicted in FIGS. 1 and 2. In embodiments, the lentiviral vector comprising PAH is the lentiviral vector expressing PAH depicted in FIG. 3.

In embodiments, the viral vector comprises a prothrombin enhancer, a hAAT promoter, and a PAH sequence (also referred to herein as LV-Pro-hAAT-PAH or AGT323). In embodiments, the prothrombin enhancer sequence is any of the prothrombin sequences or variants described herein. In embodiments, the hAAT promoter is any of the hAAT promoter sequences or variants described herein. In embodiments, the PAH sequences are any of the PAH sequences or variants described herein.

In embodiments, the lentiviral vector is comprised of an integrated lentiviral vector. In embodiments, the integrated lentiviral vector is derived from a lentiviral vector system. In embodiments, the lentiviral vector system comprises separate plasmids encoding a rev gene and an env gene. In embodiments, the integrated lentiviral vector is derived from a 3-vector lentiviral system. In embodiments, the 3-vector lentiviral system is illustrated in FIG. 1. In embodiments, the integrated lentiviral vector is derived from a 4-vector lentiviral system. In embodiments, the 4-vector lentiviral vector system is illustrated in FIG. 2.

In embodiments, the subject is treated with adeno-associated viral (AAV) vector. In embodiments, the AAV vector comprises any of the AAV vectors disclosed herein. In embodiments, the AAV vector comprises a PAH sequence or variants thereof. In embodiments, the PAH sequence is any of the PAH sequences or variants described herein.

In embodiments, the injection is an intradermal injection. In embodiments, the injection is an intramuscular injection. In embodiments, the injection is a subcutaneous injection. In embodiments, the injection is an intravenous injection.

In embodiments, the methods described herein further comprise producing a specific titer of an integrated lentiviral vector prior to treating the subject with the lentiviral particle The specific titer is determined in a test system utilizing a cell target and lentiviral vector transduction in vitro, followed by quantitative PCR analysis of chromosomal DNA from transduced cells to measure the frequency of transduced cells and the number of integrated vector copy numbers per cell. Titer is expressed as the number of integrated copy numbers that will result from transduction with an appropriate column of lentiviral vector into an appropriate number of cells. In embodiments, the titer is between 1×10⁵ and 1×10¹⁵ integrated vector copies, for example, between 1×10⁷ and 1×10¹³ integrated vector copies, or between 1×10⁹ and 1×10¹¹ integrated vector copies. In embodiments, the titer is 1×10¹⁰ integrated vector copies.

In embodiments, producing a specific titer of an integrated lentiviral vector comprises adding a vector system to one or more cells. In embodiments, the one or more cells is a cell line. In embodiments, the cell line is a 293T cell line. In embodiments, the cell line is a HeLa cell line. In embodiments, the cell line is a CHO cell line. In embodiments, the cell line is a Hep3B cell line. Persons skilled in the art will also appreciate that in other embodiments, the cell line may be any suitable cell line known in the art.

In embodiments, the method further comprises measuring the levels of Phe in the blood following injection of the lentiviral vector comprising PA.

In an aspect of the disclosure, human PAH is expressed in cells using an AAV-delivered expression system. In embodiments a AAV-2 serotype is used. In embodiments, a AAV-DJ serotype is used. In embodiments, the AAV vector contains GFP. In embodiments, the AAV vector may represent any serotype or may be generated by recombinant DNA or other synthetic approaches designed to improve transduction of human hepatocytes.

In embodiments, a human PAH is introduced into an AAV vector. In embodiments, a prothrombin enhancer is introduced into an AAV vector. In embodiments, a hAAT promoter is introduced into a AAV vector. In embodiments, a rabbit globin intron is introduced into a AAV vector. In embodiments, any one or more of a human PAH, a prothrombin enhancer, a hAAT promoter, and rabbit globin intron are introduced into an AAV vector. In embodiments the viral vector comprises a prothrombin enhancer, a hAAT promoter, and a PAH sequence (AAV-Pro-hAAT-PAH; AGT323).

In embodiments, the prothrombin enhancer sequence is any of the prothrombin sequences or variants disclosed herein. In embodiments, the PAH sequence is any of the PAH sequences or variants described herein. In embodiments, the hAAT sequence is any of the hAAT sequences or variants disclosed herein. In embodiments, the intron sequence is any of the intron sequences or variants disclosed herein.

In an aspect of the disclosure, lentiviral vector therapy is used in treatment of a subject that has a mutant PAH gene. In embodiments, the subject is a human. In other embodiments, as shown experimentally herein, the subject is a neonatal mouse derived from a Pah mutant mouse line. In embodiments, the mutant mouse line is Pah^{enu1}. In embodiments, the mutant mouse line is Pah^{enu2}. In embodiments, the mutant mouse line is Pah^{enu3}.

In embodiments, the PAH sequence in the lentiviral vector is any of the PAH sequences or variants described herein and including those listed in the PAHvdb, BIODEF, BIOPKU, JAKE or PNDdb databases found at www.biopku.org.

In embodiments, the lentiviral vector is comprised of an integrated lentiviral vector. In embodiments, the integrated lentiviral vector is derived from a lentiviral vector system. In embodiments, the lentiviral vector system comprises separate plasmids encoding a rev gene and a envelope gene. In embodiments, the integrated lentiviral vector is derived from a 3-vector lentiviral system. In embodiments, the 3-vector lentiviral system is illustrated in FIG. 1. In embodiments, the integrated lentiviral vector is derived from a 4-vector lentiviral system. In embodiments, the 4-vector lentiviral vector system is illustrated in FIG. 2.

In an aspect of the disclosure, expression of a lentivirus in cells containing a shRNA and PAH suppresses expression of endogenous PAH, but does not suppress expression of exogenous PAH that is expressed from the lentiviral vector.

In embodiments, a lentivirus containing shRNA and PAH is expressed in a subject in vivo as described herein. In embodiments, the subject is a mammal. In embodiments, the mammal is a human.

In embodiments, a lentivirus containing shRNA and PAH is expressed in vitro or ex vivo. In embodiments, the lentivirus is expressed in vitro, for example in a cell line. In embodiments, the cell line is any of the cell lines described herein or those known to those persons skilled in the relevant art. In embodiments, the cell line is a Hep3B cell line.

In embodiments, a lentiviral vector is provided comprising a prothrombin enhancer, a hAAT promoter, a PAH sequence, and a shRNA that targets endogenous PAH (optionally referred to herein as: LV-Pro-hAAT-PAH-shPAH). In embodiments, the shRNA targets the 3′UTR of endogenous PAN.

In embodiments, the prothrombin enhancer sequence comprises any of the prothrombin sequences or variants disclosed herein. In embodiments, the hAAT promoter comprises any of the hAAT promoter sequences or variants disclosed herein. In embodiments, the PAH sequence comprises any of the PAH sequences or variants described herein. In embodiments, the shRNA sequence in the lentiviral vector comprises SEQ ID NO: 13. In embodiments, the shRNA sequence in the lentiviral vector comprises SEQ ID NO: 14.

Other aspects and advantages of the inventions described herein will become apparent from the following detailed description, taken in conjunction with the accompanying drawings, which illustrate by way of example the aspects of the inventions.

### Phenylketonuria

PKU is believed to be caused by mutations of PAH and/or a defect in the synthesis or regeneration of PAH cofactors (i.e., BH₄). Notably, several PAH mutations have been shown to affect protein folding in the endoplasmic reticulum resulting in accelerated degradation and/or aggregation due to missense mutations (63%) and small deletions (13%) in protein structure that attenuates or largely abolishes enzyme catalytic activity. As there are numerous mutations that can affect the functionality of PAH, an effective therapeutic approach for treating PKU will need to address the aberrant PAH and a mode by which replacement PAH can be administered.

In general, three major phenotypic groups are classified in PKU based on Phe levels measured at diagnosis, dietary tolerance to Phe and potential responsiveness to therapy. These groups include classical PKU (Phe>1200 μM), atypical or mild PKU (Phe is 600-1200 μM), and permanent mild hyperphenylalaninemia (HPA, Phe 120-600 μM).

Detection of PKU relies on universal newborn screening (NBS). A drop of blood collected from a heel stick is tested for phenylalanine levels in a screen that is mandatory in all 50 states of the USA and used routinely in most developed countries.

### Genetic Medicines

Genetic medicine includes reference to viral vectors that are used to deliver genetic constructs to host cells for the purposes of disease therapy or prevention.

Genetic constructs can include, but are not limited to, functional genes or portions of genes to correct or complement existing defects, DNA sequences encoding regulatory proteins, DNA sequences encoding regulatory RNA molecules including antisense, short hairpin RNA, short homology RNA, long non-coding RNA, small interfering RNA or others, and decoy sequences encoding either RNA or proteins designed to compete for critical cellular factors to alter a disease state. Genetic medicine involves delivering these therapeutic genetic constructs to target cells to provide treatment or alleviation of a particular disease.

By delivering a functional PAH gene to the liver in vivo, PAH activity should be reconstituted leading to normal clearance of Phe in the blood therefore eliminating the need for dietary restrictions or frequent enzyme replacement therapies. The effect of this therapeutic approach should be improved by the targeting of a shRNA against endogenous PA. In an aspect of the disclosure, a functional PAH gene or a variant thereof can also be delivered in utero if a fetus has been identified as being at risk to a PKU genotype. In embodiments, the diagnostic step can be carried out to determine whether the fetus is at risk for a PKU phenotype. If the diagnostic step determines that the fetus is at risk for a PKU phenotype, then the fetus can be treated with the genetic medicines detailed herein. Treatment can occur in utero or in vitro.

### Therapeutic Vectors

A lentiviral virion (particle) in accordance with various aspects and embodiments herein is expressed by a vector system encoding the necessary viral proteins to produce a virion (viral particle). In various embodiments, one vector containing a nucleic acid sequence encoding the lentiviral Pol proteins is provided for reverse transcription and integration, operably linked to a promoter. In another embodiment, the Pol proteins are expressed by multiple vectors. In other embodiments, vectors containing a nucleic acid sequence encoding the lentiviral Gag proteins for forming a viral capsid, operably linked to a promoter, are provided. In embodiments, this gag nucleic acid sequence is on a separate vector than at least some of the pol nucleic acid sequence.

Numerous modifications can be made to the vectors herein, which are used to create the particles to further minimize the chance of obtaining wild type revertants. These include, but are not limited to deletions of the U3 region of the LTR, tat deletions and matrix (MA) deletions. In embodiments, the gag, pol and env vector(s) do not contain nucleotides from the lentiviral genome that package lentiviral RNA, referred to as the lentiviral packaging sequence.

The vector(s) forming the particle preferably do not contain a nucleic acid sequence from the lentiviral genome that expresses an envelope protein. Preferably, a separate vector that contains a nucleic acid sequence encoding an envelope protein operably linked to a promoter is used. This env vector also does not contain a lentiviral packaging sequence. In one embodiment the env nucleic acid sequence encodes a lentiviral envelope protein.

In another embodiment the envelope protein is not from the lentivirus, but from a different virus. The resultant particle is referred to as a pseudotyped particle. By appropriate selection of envelope proteins one can infect virtually any cell. For example, one can use an env gene that encodes an envelope protein that targets an endocytic compartment such as that of the influenza virus, VSV-G or similar envelope proteins from human and nonhuman rhabdovirus isolates, alpha viruses (Semliki forest virus, Sindbis virus), arenaviruses (lymphocytic choriomeningitis virus), flaviviruses (tick-bome encephalitis virus, Dengue virus, hepatitis C virus, GB virus), rhabdoviruses (vesicular stomatitis virus, rabies virus), paramxoviruses (mumps or measles) and orthomyxoviruses (influenza virus). Other envelope proteins that can preferably be used include those from Feline Leukemia Virus and feline endogenous retroviruses, Moloney Leukemia Virus such as MLV-E, MLV-A, Gibbon Ape Leukemia Virus GALV, and Baboon Endogenous Retrovirus. These latter envelope proteins are particularly preferred where the host cell is a primary cell. Other envelope proteins can be selected depending upon the desired host cell.

Lentiviral vector systems as provided herein typically include at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 1). In another embodiment, the gag and pol genes are provided on a first plasmid and the rev gene is provided on a second plasmid (e.g., FIG. 2). Accordingly, both 3-vector and 4-vector systems can be used to produce a lentivirus as described herein. In embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting of producing PAH and/or inhibiting the expression of endogenous PAH.

In another aspect, the lentiviral vector, which is also referred to herein as a therapeutic vector, includes the following elements: hybrid 5′ long terminal repeat (RSV/5′ LTR) (SEQ ID NOs: 15-16), Psi sequence (RNA packaging site) (SEQ ID NO: 17), RRE (Rev-response element) (SEQ ID NO: 18), cPPT (polypurine tract) (SEQ ID NO: 19), Anti alpha trypsin promoter (hAAT) (SEQ ID NO: 6), Phenylalanine hydroxylase (PAH) (SEQ ID NOs: 1-4, Woodchuck Post-Transcriptional Regulatory Element (WPRE) (SEQ ID NOs: 20), and ΔU3 3′ LTR (SEQ ID NO: 21). In embodiments, the lentiviral vector, which is also referred to herein as a therapeutic vector, includes the following elements: hybrid 5′ long terminal repeat (RSV/5′ LTR) (SEQ ID NOs: 15-16), Psi sequence (RNA packaging site) (SEQ ID NO: 17), RRE (Rev-response element) (SEQ ID NO: 18), cPPT (polypurine tract) (SEQ ID NO: 19), H1 promoter (SEQ ID NO: 22), PAH shRNA (SEQ ID NOs: 1-4), Anti alpha trypsin promoter (hAAT) (SEQ ID NO: 6), PAH shRNA (SEQ ID NOs: 1-4), Woodchuck Post-Transcriptional Regulatory Element (WPRE) (SEQ ID NO: 20), and ΔU3 3′ LTR (SEQ ID NO: 21). In embodiments, sequence variation, by way of substitution, deletion, addition, or mutation can be used to modify the sequences references herein.

In another aspect, a helper plasmid includes the following elements: CMV enhancer/chicken beta actin enhancer (SEQ ID NO: 23); HIV component gag (SEQ ID NO: 24); HIV component pol (SEQ ID NO: 25); HIV Int (SEQ ID NO: 26); HIV RRE (SEQ ID NO: 27); and HIV Rev (SEQ ID NO: 28). In another aspect, the helper plasmid may be modified to include a first helper plasmid for expressing the gag and pol genes, and a second and separate plasmid for expressing the rev gene. In embodiments, sequence variation, by way of substitution, deletion, addition, or mutation can be used to modify the sequences references herein.

In another aspect, an envelope plasmid includes the following elements: RNA polymerase II promoter (CMV) (SEQ ID NO: 29) and vesicular stomatitis virus G glycoprotein (VSV-G) (SEQ ID NO: 30). In embodiments, sequence variation, by way of substitution, deletion, addition, or mutation can be used to modify the sequences references herein.

In various aspects, the plasmids used for lentiviral packaging are modified by substitution, addition, subtraction or mutation of various elements without loss of vector function. For example, and without limitation, the following elements can replace similar elements in the plasmids that comprise the packaging system: Elongation Factor-1 (EF-1), phosphoglycerate kinase (PGK), and ubiquitin C (UbC) promoters can replace the CMV or CAG promoter. SV40 poly A and bGH poly A can replace the rabbit beta globin poly A. The HIV sequences in the helper plasmid can be constructed from different HIV strains or clades. The VSV-G glycoprotein can be substituted with membrane glycoproteins from human endogenous retroviruses including HERV-W, baboon endogenous retrovirus BaEV, feline endogenous virus (RD114), gibbon ape leukemia virus (GALV), Rabies (FUG), lymphocytic choriomeningitis virus (LCMV), influenza A fowl plague virus (FPV), Ross River alphavirus (RRV), murine leukemia virus 10A1 (MLV), or Ebola virus (EboV).

Various lentiviral packaging systems can be acquired commercially (e.g., Lenti-vpak packaging kit from OriGene Technologies, Inc., Rockville, Md.), and can also be designed as described herein. Moreover, it is within the skill of a person ordinarily skilled in the relevant art to substitute or modify aspects of a lentiviral packaging system to improve any number of relevant factors, including the production efficiency of a lentiviral particle.

In another aspect, adeno-associated viral (AAV) vectors can also be used. In embodiments, the AAV vector is an AAV-DJ serotype. In embodiments, the AAV vector is any of serotypes 1-11. In embodiments, the AAV serotype is AAV-2. In embodiments, the AAV vector is a non-natural type engineered for optimal transduction of human hepatocytes.

AAV Vector Construction.

In aspects of the disclosure, the PAH coding sequence (SEQ ID NOs: 1-4) and the prothrombin enhancer (SEQ ID NO: 5) with hAAT promoter (SEQ ID NO: 6) are inserted into the pAAV plasmid (Cell Biolabs, San Diego, Calif.). The PAH coding sequence with flanking EcoRI and SalI restriction sites is synthesized by Eurofins Genomics (Louisville, Ky.). The pAAV plasmid and PAH sequence are digested with EcoRI and SalI enzyme and ligated together. Insertion of the PAH sequence is verified by sequencing. Next, the prothrombin enhancer and hAAT promoter are synthesized by Eurofins Genomics (Louisville, Ky.) with flanking MluI and EcoRI restriction sites. The pAAV plasmid containing the PAH coding sequence and the prothrombin enhancer/hAAT promoter sequence are digested with MluI and EcoRI enzymes and ligated together. Insertion of the prothrombin enhancer/hAAT promoter are verified by sequencing.

Further, a representative AAV plasmid system for expressing PAH may comprise an AAV Helper plasmid, an AAV plasmid, and an AAV Rev/Cap plasmid. The AAV Helper plasmid may contain a Left ITR (SEQ ID NO: 31), a Prothrombin enhancer (SEQ ID NO: 5), a human Anti alpha trypsin promoter (SEQ ID NO: 6), a PAH element (SEQ ID NOs: 1-4), a PolyA element (SEQ ID NO: 32), and a Right ITR (SEQ ID NO: 33). The AAV plasmid may contain a suitable promoter element (SEQ ID NO: 23 or SEQ ID NO: 29), an E2A element (SEQ ID NO: 34), an E4 element (SEQ ID NO: 35), a VA RNA element (SEQ ID NO: 36), and a PolyA element (SEQ ID NO: 32). The AAV Rep/Cap plasmid may contain a suitable promoter element, a Rep element (SEQ ID NO: 37), a Cap element (SEQ ID NO: 38), and a PolyA element (SEQ ID NO: 32).

In embodiments, an AAV/DJ plasmid is provided comprising a prothrombin enhancer and a PAH sequence (AAV/DJ-Pro-PAH). In embodiments, an AAV/DJ plasmid is provided comprising a prothrombin enhancer, an intron, and a PAH sequence (AAV/DJ-Pro-Intron-PAH). In embodiments, the intron is a human beta globin intron. In embodiments, the intron is a rabbit beta globin intron. In embodiments, an AAV/DJ plasmid is provided comprising GFP (AAV/DJ-GFP).

In embodiments, an AAV2 plasmid is provided comprising a prothrombin enhancer and a PAH sequence (AAV2-Pro-PAH). In embodiments, an AAV2 plasmid is provided comprising a prothrombin enhancer, an intron, and a PAH sequence (AAV2-Pro-Intron-PAH). In embodiments, the intron is a human beta globin intron. In embodiments, the intron is a rabbit beta globin intron. In embodiments, an AAV2 is provided comprising GFP (AAV2-GFP).

In embodiments, any of the AAV vectors disclosed herein may contain a sequence that expresses a regulatory RNA. In embodiments, the regulatory RNA is a lncRNA. In embodiments, the regulatory RNA is a microRNA. In embodiments, the regulatory RNA is a piRNA. In embodiments, the regulatory RNA is a shRNA. In embodiments, the regulatory RNA is a small RNA sequence comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NOs: 13 or 14.

Production of AAV Particles.

The AAV-PAH plasmid is combined with the plasmids pAAV-RC2 (Cell Biolabs) and pHelper (Cell Biolabs). The pAAV-RC2 plasmid contains the Rep and AAV-2 capsid genes and pHelper contains the adenovirus E2A, E4, and VA genes. The AAV capsid may also consist of the AAV-8 (SEQ ID NO: 39) or AAV-DJ (SEQ ID NO: 40) sequences. To produce AAV particles, these plasmids are transfected in the ratio 1:1:1 (pAAV-PAH:pAAV-RC2:pHelper) into 293T cells. For transfection of cells in 150 mm dishes (BD Falcon), 10 micrograms of each plasmid are added together in 1 ml of DMEM. In another tube, 60 microliters of the transfection reagent PEI (1 microgram/ml) (Polysciences) is added to 1 ml of DMEM. The two tubes are mixed together and allowed to incubate for 15 minutes. Then the transfection mixture is added to cells and the cells are collected after 3 days. The cells are lysed by freeze/thaw lysis in dry ice/isopropanol. Benzonase nuclease (Sigma) is added to the cell lysate for 30 minutes at 37 degrees Celsius. Cell debris are then pelleted by centrifugation at 4 degrees Celsius for 15 minutes at 12,000 rpm. The supernatant is collected and then added to target cells.

### Dosage and Dosage Forms

The disclosed vector compositions allow for short, medium, or long-term expression of genes or sequences of interest and episomal maintenance of the disclosed vectors. Accordingly, dosing regimens may vary based upon the condition being treated and the method of administration.

In embodiments, vector compositions may be administered to a subject in need in varying doses. Specifically, a subject may be administered about ≥10⁶ infectious doses (where 1 dose is needed on average to transduce 1 target cell). More specifically, a subject may be administered about ≥10⁷, about ≥10⁸, about ≥10⁹, about ≥10¹⁰, about ≥10¹¹ or about ≥10¹² infectious doses per kilogram of body weight, or any number of doses in-between these values. Upper limits of dosing will be determined for each disease indication, and will depend on toxicity/safety profiles for each individual product or product lot.

Additionally, vector compositions of the present disclosure may be administered periodically, such as once or twice a day, or any other suitable time period. For example, vector compositions may be administered to a subject in need once a week, once every other week, once every three weeks, once a month, every other month, every three months, every six months, every nine months, once a year, every eighteen months, every two years, every thirty months, or every three years.

In embodiments, the disclosed vector compositions are administered as a pharmaceutical composition. In embodiments, the pharmaceutical composition can be formulated in a wide variety of dosage forms, including but not limited to nasal, pulmonary, oral, topical, or parenteral dosage forms for clinical application. Each of the dosage forms can comprise various solubilizing agents, disintegrating agents, surfactants, fillers, thickeners, binders, diluents such as wetting agents or other pharmaceutically acceptable excipients. The pharmaceutical composition can also be formulated for injection, insufflation, infusion, or intradermal exposure. For instance, an injectable formulation may comprise the disclosed vectors in an aqueous or non-aqueous solution at a suitable pH and tonicity.

The disclosed vector compositions may be administered to a subject via direct injection into the liver with guided injection. In some embodiments, the vectors can be administered systemically via arterial or venous circulation. In some embodiments, the vector compositions can be administered via guided cannulation to tissues immediately surrounding liver including spleen or pancreas. In some embodiments, the vector composition may be delivered by injection into the portal vein or portal sinus, and may be delivered by injection into the umbilical vein.

The disclosed vector compositions can be administered using any pharmaceutically acceptable method, such as intranasal, buccal, sublingual, oral, rectal, ocular, parenteral (intravenously, intradermally, intramuscularly, subcutaneously, intraperitoneally), pulmonary, intravaginal, locally administered, topically administered, topically administered after scarification, mucosally administered, via an aerosol, in semi-solid media such as agarose or gelatin, or via a buccal or nasal spray formulation.

Further, the disclosed vector compositions can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, a solution, an emulsion, and a suspension. Further, the pharmaceutical composition may be a controlled release formulation, sustained release formulation, immediate release formulation, or any combination thereof. Further, the pharmaceutical composition may be a transdermal delivery system.

In embodiments, the pharmaceutical composition can be formulated in a solid dosage form for oral administration, and the solid dosage form can be powders, granules, capsules, tablets or pills. In embodiments, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In embodiments, the pharmaceutical composition can be formulated as a sublingual or buccal dosage form. Such dosage forms comprise sublingual tablets or solution compositions that are administered under the tongue and buccal tablets that are placed between the cheek and gum.

In embodiments, the pharmaceutical composition can be formulated as a nasal dosage form. Such dosage forms of this disclosure comprise solution, suspension, and gel compositions for nasal delivery.

In embodiments, the pharmaceutical composition can be formulated in a liquid dosage form for oral administration, such as suspensions, emulsions or syrups. In embodiments, the liquid dosage form can include, in addition to commonly used simple diluents such as water and liquid paraffin, various excipients such as humectants, sweeteners, aromatics or preservatives. In embodiments, the composition can be formulated to be suitable for administration to a pediatric patient.

In embodiments, the pharmaceutical composition can be formulated in a dosage form for parenteral administration, such as sterile aqueous solutions, suspensions, emulsions, non-aqueous solutions or suppositories. In embodiments, the solutions or suspensions can include propyleneglycol, polyethyleneglycol, vegetable oils such as olive oil or injectable esters such as ethyl oleate.

The dosage of the pharmaceutical composition can vary depending on the patient's weight, age, gender, administration time and mode, excretion rate, and the severity of disease.

In embodiments, the treatment of PKU is accomplished by guided direct injection of the disclosed vector constructs into liver, using needle, or intravascular cannulation. In embodiments, the vectors compositions are administered into the cerebrospinal fluid, blood or lymphatic circulation by venous or arterial cannulation or injection, intradermal delivery, intramuscular delivery or injection into a draining organ near the liver.

The following examples are given to illustrate aspects of the present invention. It should be understood, however, that the inventions are not to be limited to the specific conditions or details described in these examples. All printed publications referenced herein are specifically incorporated by reference.

### EXAMPLES

### Example 1. Development of a Lentiviral Vector System

A lentiviral vector system was developed as summarized in FIG. 1 (circularized form). Lentiviral particles were produced in 293T/17 HEK cells (purchased from American Type Culture Collection, Manassas, Va.) following transfection with the therapeutic vector, the envelope plasmid, and the helper plasmid. The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), measuring the number of viral DNA copies per transduced cell by quantitative PCR, or by infecting cells and using light (if the vectors encode luciferase or fluorescent protein markers).

As mentioned above, a 3-vector system (i.e., which includes a 2-vector lentiviral packaging system) was designed for the production of lentiviral particles. A schematic of the 3-vector system is shown in FIG. 1. Briefly, and with reference to FIG. 1, the top-most vector is a helper plasmid, which, in this case, includes Rev. The vector appearing in the middle of FIG. 1 is the envelope plasmid. The bottom-most vector is the therapeutic vector, as described herein.

Referring to FIG. 1, the Helper plus Rev plasmid includes a CMV enhancer with chicken beta actin promoter (SEQ ID NO: 23); a chicken beta actin intron (SEQ ID NO: 41); a HIV Gag (SEQ ID NO: 24); a HIV Pol (SEQ ID NO: 25); a HIV Integrase (SEQ ID NO: 26); a HIV RRE (SEQ ID NO: 27); a HIV Rev (SEQ ID NO: 28); and a rabbit beta globin poly A (SEQ ID NO: 42).

The Envelope plasmid includes a CMV promoter (SEQ ID NO: 29); a beta globin intron (SEQ ID NO: 7 or 8); a VSV-G envelope glycoprotein (SEQ ID NO: 30); and a rabbit beta globin poly A (SEQ ID NO: 42).

Synthesis of a 3-vector system, which includes a 2-vector lentiviral packaging system consisting of Helper (plus Rev) and Envelope plasmids, is disclosed.

Materials and Methods:

Construction of the Helper Plasmid:

The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and Nod restriction sites which could be used to insert at the same sites in the pCDNA3 plasmid (Invitrogen). The forward primer was (5′-TAAGCAGAATTCATGAATTTGCCAGGAAGAT-3′) (SEQ ID NO: 43) and reverse primer was (5′-CCATACAATGAATGGACACTAGGCGGCCGCACGAAT-3′) (SEQ ID NO: 44).

The sequence for the Gag, Pol, Integrase fragment was as follows:

(SEQ ID NO: 45)
GAATTCATGAATTTGCCAGGAAGATGGAAACCAAAAATGATAGGGGGA
ATTGGAGGTTTTATCAAAGTAAGACAGTATGATCAGATACTCATAGAA
ATCTGCGGACATAAAGCTATAGGTACAGTATTAGTAGGACCTACACCT
GTCAACATAATTGGAAGAAATCTGTTGACTCAGATTGGCTGCACTTTA
AATTTTCCCATTAGTCCTATTGAGACTGTACCAGTAAAATTAAAGCCA
GGAATGGATGGCCCAAAAGTTAAACAATGGCCATTGACAGAAGAAAAA
ATAAAAGCATTAGTAGAAATTTGTACAGAAATGGAAAAGGAAGGAAAA
ATTTCAAAAATTGGGCCTGAAAATCCATACAATACTCCAGTATTTGCC
ATAAAGAAAAAAGACAGTACTAAATGGAGAAAATTAGTAGATTTCAGA
GAACTTAATAAGAGAACTCAAGATTTCTGGGAAGTTCAATTAGGAATA
CCACATCCTGCAGGGTTAAAACAGAAAAAATCAGTAACAGTACTGGAT
GTGGGCGATGCATATTTTTCAGTTCCCTTAGATAAAGACTTCAGGAAG
TATACTGCATTTACCATACCTAGTATAAACAATGAGACACCAGGGATT
AGATATCAGTACAATGTGCTTCCACAGGGATGGAAAGGATCACCAGCA
ATATTCCAGTGTAGCATGACAAAAATCTTAGAGCCTTTTAGAAAACAA
AATCCAGACATAGTCATCTATCAATACATGGATGATTTGTATGTAGGA
TCTGACTTAGAAATAGGGCAGCATAGAACAAAAATAGAGGAACTGAGA
CAACATCTGTTGAGGTGGGGATTTACCACACCAGACAAAAAACATCAG
AAAGAACCTCCATTCCTTTGGATGGGTTATGAACTCCATCCTGATAAA
TGGACAGTACAGCCTATAGTGCTGCCAGAAAAGGACAGCTGGACTGTC
AATGACATACAGAAATTAGTGGGAAAATTGAATTGGGCAAGTCAGATT
TATGCAGGGATTAAAGTAAGGCAATTATGTAAACTTCTTAGGGGAACC
AAAGCACTAACAGAAGTAGTACCACTAACAGAAGAAGCAGAGCTAGAA
CTGGCAGAAAACAGGGAGATTCTAAAAGAACCGGTACATGGAGTGTAT
TATGACCCATCAAAAGACTTAATAGCAGAAATACAGAAGCAGGGGCAA
GGCCAATGGACATATCAAATTTATCAAGAGCCATTTAAAAATCTGAAA
ACAGGAAAGTATGCAAGAATGAAGGGTGCCCACACTAATGATGTGAAA
CAATTAACAGAGGCAGTACAAAAAATAGCCACAGAAAGCATAGTAATA
TGGGGAAAGACTCCTAAATTTAAATTACCCATACAAAAGGAAACATGG
GAAGCATGGTGGACAGAGTATTGGCAAGCCACCTGGATTCCTGAGTGG
GAGTTTGTCAATACCCCTCCCTTAGTGAAGTTATGGTACCAGTTAGAG
AAAGAACCCATAATAGGAGCAGAAACTTTCTATGTAGATGGGGCAGCC
AATAGGGAAACTAAATTAGGAAAAGCAGGATATGTAACTGACAGAGGA
AGACAAAAAGTTGTCCCCCTAACGGACACAACAAATCAGAAGACTGAG
TTACAAGCAATTCATCTAGCTTTGCAGGATTCGGGATTAGAAGTAAAC
ATAGTGACAGACTCACAATATGCATTGGGAATCATTCAAGCACAACCA
GATAAGAGTGAATCAGAGTTAGTCAGTCAAATAATAGAGCAGTTAATA
AAAAAGGAAAAAGTCTACCTGGCATGGGTACCAGCACACAAAGGAATT
GGAGGAAATGAACAAGTAGATAAATTGGTCAGTGCTGGAATCAGGAAA
GTACTATTTTTAGATGGAATAGATAAGGCCCAAGAAGAACATGAGAAA
TATCACAGTAATTGGAGAGCAATGGCTAGTGATTTTAACCTACCACCT
GTAGTAGCAAAAGAAATAGTAGCCAGCTGTGATAAATGTCAGCTAAAA
GGGGAAGCCATGCATGGACAAGTAGACTGTAGCCCAGGAATATGGCAG
CTAGATTGTACACATTTAGAAGGAAAAGTTATCTTGGTAGCAGTTCAT
GTAGCCAGTGGATATATAGAAGCAGAAGTAATTCCAGCAGAGACAGGG
CAAGAAACAGCATACTTCCTCTTAAAATTAGCAGGAAGATGGCCAGTA
AAAACAGTACATACAGACAATGGCAGCAATTTCACCAGTACTACAGTT
AAGGCCGCCTGTTGGTGGGCGGGGATCAAGCAGGAATTTGGCATTCCC
TACAATCCCCAAAGTCAAGGAGTAATAGAATCTATGAATAAAGAATTA
AAGAAAATTATAGGACAGGTAAGAGATCAGGCTGAACATCTTAAGACA
GCAGTACAAATGGCAGTATTCATCCACAATTTTAAAAGAAAAGGGGGG
ATTGGGGGGTACAGTGCAGGGGAAAGAATAGTAGACATAATAGCAACA
GACATACAAACTAAAGAATTACAAAAACAAATTACAAAAATTCAAAAT
TTTCGGGTTTATTACAGGGACAGCAGAGATCCAGTTTGGAAAGGACCA
GCAAAGCTCCTCTGGAAAGGTGAAGGGGCAGTAGTAATACAAGATAAT
AGTGACATAAAAGTAGTGCCAAGAAGAAAAGCAAAGATCATCAGGGAT
TATGGAAAACAGATGGCAGGTGATGATTGTGTGGCAAGTAGACAGGAT
GAGGATTAA.

Next, a DNA fragment containing the RRE, Rev, and rabbit beta globin poly A sequence with XbaI and XmaI flanking restriction sites was synthesized by Eurofins Genomics. The DNA fragment was then inserted into the plasmid at the XbaI and XmaI restriction sites The DNA sequence was as follows:

(SEQ ID NO: 46)
TCTAGAATGGCAGGAAGAAGCGGAGACAGCGACGAAGAGCTCATCAGA
ACAGTCAGACTCATCAAGCTTCTCTATCAAAGCAACCCACCTCCCAAT
CCCGAGGGGACCCGACAGGCCCGAAGGAATAGAAGAAGAAGGTGGAGA
GAGAGACAGAGACAGATCCATTCGATTAGTGAACGGATCCTTGGCACT
TATCTGGGACGATCTGCGGAGCCTGTGCCTCTTCAGCTACCACCGCTT
GAGAGACTTACTCTTGATTGTAACGAGGATTGTGGAACTTCTGGGACG
CAGGGGGTGGGAAGCCCTCAAATATTGGTGGAATCTCCTACAATATTG
GAGTCAGGAGCTAAAGAATAGAGGAGCTTTGTTCCTTGGGTTCTTGGG
AGCAGCAGGAAGCACTATGGGCGCAGCGTCAATGACGCTGACGGTACA
GGCCAGACAATTATTGTCTGGTATAGTGCAGCAGCAGAACAATTTGCT
GAGGGCTATTGAGGCGCAACAGCATCTGTTGCAACTCACAGTCTGGGG
CATCAAGCAGCTCCAGGCAAGAATCCTGGCTGTGGAAAGATACCTAAA
GGATCAACAGCTCCTAGATCTTTTTCCCTCTGCCAAAAATTATGGGGA
CATCATGAAGCCCCTTGAGCATCTGACTTCTGGCTAATAAAGGAAATT
TATTTTCATTGCAATAGTGTGTTGGAATTTTTTGTGTCTCTCACTCGG
AAGGACATATGGGAGGGCAAATCATTTAAAACATCAGAATGAGTATTT
GGTTTAGAGTTTGGCAACATATGCCATATGCTGGCTGCCATGAACAAA
GGTGGCTATAAAGAGGTCATCAGTATATGAAACAGCCCCCTGCTGTCC
ATTCCTTATTCCATAGAAAAGCCTTGACTTGAGGTTAGATTTTTTTTA
TATTTTGTTTTGTGTTATTTTTTTCTTTAACATCCCTAAAATTTTCCT
TACATGTTTTACTAGCCAGATTTTTCCTCCTCTCCTGACTACTCCCAG
TCATAGCTGTCCCTCTTCTCTTATGAAGATCCCTCGACCTGCAGCCCA
AGCTTGGCGTAATCATGGTCATAGCTGTTTCCTGTGTGAAATTGTTAT
CCGCTCACAATTCCACACAACATACGAGCCGGAAGCATAAAGTGTAAA
GCCTGGGGTGCCTAATGAGTGAGCTAACTCACATTAATTGCGTTGCGC
TCACTGCCCGCTTTCCAGTCGGGAAACCTGTCGTGCCAGCGGATCCGC
ATCTCAATTAGTCAGCAACCATAGTCCCGCCCCTAACTCCGCCCATCC
CGCCCCTAACTCCGCCCAGTTCCGCCCATTCTCCGCCCCATGGCTGAC
TAATTTTTTTTATTTATGCAGAGGCCGAGGCCGCCTCGGCCTCTGAGC
TATTCCAGAAGTAGTGAGGAGGCTTTTTTGGAGGCCTAGGCTTTTGCA
AAAAGCTAACTTGTTTATTGCAGCTTATAATGGTTACAAATAAAGCAA
TAGCATCACAAATTTCACAAATAAAGCATTTTTTTCACTGCATTCTAG
TTGTGGTTTGTCCAAACTCATCAATGTATCTTATCAGCGGCCGCCCCG
GG

Finally, the CMV promoter of pCDNA3.1 was replaced with the CAG promoter (CMV enhancer, chicken beta actin promoter plus a chicken beta actin intron sequence). A DNA fragment containing the CAG enhancer/promoter/intron sequence with MluI and EcoRI flanking restriction sites was synthesized by Eurofins Genomics. The DNA fragment was then inserted into the plasmid at the MluI and EcoRI restriction sites. The DNA sequence was as follows:

(SEQ ID NO: 47)
ACGCGTTAGTTATTAATAGTAATCAATTACGGGGTCATTAGTTCATAG
CCCATATATGGAGTTCCGCGTTACATAACTTACGGTAAATGGCCCGCC
TGGCTGACCGCCCAACGACCCCCGCCCATTGACGTCAATAATGACGTA
TGTTCCCATAGTAACGCCAATAGGGACTTTCCATTGACGTCAATGGGT
GGACTATTTACGGTAAACTGCCCACTTGGCAGTACATCAAGTGTATCA
TATGCCAAGTACGCCCCCTATTGACGTCAATGACGGTAAATGGCCCGC
CTGGCATTATGCCCAGTACATGACCTTATGGGACTTTCCTACTTGGCA
GTACATCTACGTATTAGTCATCGCTATTACCATGGGTCGAGGTGAGCC
CCACGTTCTGCTTCACTCTCCCCATCTCCCCCCCCTCCCCACCCCCAA
TTTTGTATTTATTTATTTTTTAATTATTTTGTGCAGCGATGGGGGCGG
GGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCG
CTCCGAAAGTTTCCTTTTATGGCGAGGCGGCGGCGGCGGCGGCCCTAT
AAAAAGCGAAGCGCGCGGCGGGCGGGAGTCGCTGCGTTGCCTTCGCCC
CGTGCCCCGCTCCGCGCCGCCTCGCGCCGCCCGCCCCGGCTCTGACTG
ACCGCGTTACTCCCACAGGTGAGCGGGCGGGACGGCCCTTCTCCTCCG
GGCTGTAATTAGCGCTTGGTTTAATGACGGCTCGTTTCTTTTCTGTGG
CTGCGTGAAAGCCTTAAAGGGCTCCGGGAGGGCCCTTTGTGCGGGGGG
GAGCGGCTCGGGGGGTGCGTGCGTGTGTGTGTGCGTGGGGAGCGCCGC
GTGCGGCCCGCGCTGCCCGGCGGCTGTGAGCGCTGCGGGCGCGGCGCG
GGGCTTTGTGCGCTCCGCGTGTGCGCGAGGGGAGCGCGGCCGGGGGCG
GTGCCCCGCGGTGCGGGGGGGCTGCGAGGGGAACAAAGGCTGCGTGCG
GGGTGTGTGCGTGGGGGGGTGAGCAGGGGGTGTGGGCGCGGCGGTCGG
GCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCG
TGCCGGGCGGGGGGTGGCGGCAGGTGGGGGTGCCGGGCGGGGCGGGGC
CGCCTCGGGCCGGGGAGGGCTCGGGGGAGGGGCGCGGCGGCCCCGGAG
CGCCGGCGGCTGTCGAGGCGCGGCGAGCCGCAGCCATTGCCTTTTATG
GTAATCGTGCGAGAGGGCGCAGGGACTTCCTTTGTCCCAAATCTGGCG
GAGCCGAAATCTGGGAGGCGCCGCCGCACCCCCTCTAGCGGGCGCGGG
CGAAGCGGTGCGGCGCCGGCAGGAAGGAAATGGGCGGGGAGGGCCTTC
GTGCGTCGCCGCGCCGCCGTCCCCTTCTCCATCTCCAGCCTCGGGGCT
GCCGCAGGGGGACGGCTGCCTTCGGGGGGGACGGGGCAGGGCGGGGTT
CGGCTTCTGGCGTGTGACCGGCGGGAATTC

Construction of the VSV-G Envelope Plasmid:

The vesicular stomatitis Indiana virus glycoprotein (VSV-G) sequence was synthesized by Eurofins Genomics with flanking EcoRI restriction sites. The DNA fragment was then inserted into the pCDNA3.1 plasmid (Invitrogen) at the EcoRI restriction site and the correct orientation was determined by sequencing using a CMV specific primer.

The DNA sequence was as follows:

(SEQ ID NO: 30)
ATGAAGTGCCTTTTGTACTTAGCCTTTTTATTCATTGGGGTGAATTGC
AAGTTCACCATAGTTTTTCCACACAACCAAAAAGGAAACTGGAAAAAT
GTTCCTTCTAATTACCATTATTGCCCGTCAAGCTCAGATTTAAATTGG
CATAATGACTTAATAGGCACAGCCTTACAAGTCAAAATGCCCAAGAGT
CACAAGGCTATTCAAGCAGACGGTTGGATGTGTCATGCTTCCAAATGG
GTCACTACTTGTGATTTCCGCTGGTATGGACCGAAGTATATAACACAT
TCCATCCGATCCTTCACTCCATCTGTAGAACAATGCAAGGAAAGCATT
GAACAAACGAAACAAGGAACTTGGCTGAATCCAGGCTTCCCTCCTCAA
AGTTGTGGATATGCAACTGTGACGGATGCCGAAGCAGTGATTGTCCAG
GTGACTCCTCACCATGTGCTGGTTGATGAATACACAGGAGAATGGGTT
GATTCACAGTTCATCAACGGAAAATGCAGCAATTACATATGCCCCACT
GTCCATAACTCTACAACCTGGCATTCTGACTATAAGGTCAAAGGGCTA
TGTGATTCTAACCTCATTTCCATGGACATCACCTTCTTCTCAGAGGAC
GGAGAGCTATCATCCCTGGGAAAGGAGGGCACAGGGTTCAGAAGTAAC
TACTTTGCTTATGAAACTGGAGGCAAGGCCTGCAAAATGCAATACTGC
AAGCATTGGGGAGTCAGACTCCCATCAGGTGTCTGGTTCGAGATGGCT
GATAAGGATCTCTTTGCTGCAGCCAGATTCCCTGAATGCCCAGAAGGG
TCAAGTATCTCTGCTCCATCTCAGACCTCAGTGGATGTAAGTCTAATT
CAGGACGTTGAGAGGATCTTGGATTATTCCCTCTGCCAAGAAACCTGG
AGCAAAATCAGAGCGGGTCTTCCAATCTCTCCAGTGGATCTCAGCTAT
CTTGCTCCTAAAAACCCAGGAACCGGTCCTGCTTTCACCATAATCAAT
GGTACCCTAAAATACTTTGAGACCAGATACATCAGAGTCGATATTGCT
GCTCCAATCCTCTCAAGAATGGTCGGAATGATCAGTGGAACTACCACA
GAAAGGGAACTGTGGGATGACTGGGCACCATATGAAGACGTGGAAATT
GGACCCAATGGAGTTCTGAGGACCAGTTCAGGATATAAGTTTCCTTTA
TACATGATTGGACATGGTATGTTGGACTCCGATCTTCATCTTAGCTCA
AAGGCTCAGGTGTTCGAACATCCTCACATTCAAGACGCTGCTTCGCAA
CTTCCTGATGATGAGAGTTTATTTTTTGGTGATACTGGGCTATCCAAA
AATCCAATCGAGCTTGTAGAAGGTTGGTTCAGTAGTTGGAAAAGCTCT
ATTGCCTCTTTTTTCTTTATCATAGGGTTAATCATTGGACTATTCTTG
GTTCTCCGAGTTGGTATCCATCTTTGCATTAAATTAAAGCACACCAAG
AAAAGACAGATTTATACAGACATAGAGATGAACCGACTTGGAAAGTGA

A 4-vector system, which includes a 3-vector lentiviral packaging system, has also been designed and produced using the methods and materials described herein. A schematic of the 4-vector system is shown in FIG. 2. Briefly, and with reference to FIG. 2, the top-most vector is a helper plasmid, which, in this case, does not include Rev. The second vector is a separate Rev plasmid. The third vector is the envelope plasmid. The bottom-most vector is the therapeutic vector as described herein.

Referring to FIG. 2, the Helper plasmid includes a CMV enhancer and chicken beta actin promoter (SEQ ID NO: 23); a chicken beta actin intron (SEQ ID NO: 41); a HIV Gag (SEQ ID NO: 24); a HIV Pol (SEQ ID NO: 25); a HIV Integrase (SEQ ID NO: 26); a HIV RRE (SEQ ID NO: 27); and a rabbit beta globin poly A (SEQ ID NO: 42).

The Rev plasmid includes a RSV promoter and HIV Rev (SEQ ID NO: 48); and a rabbit beta globin poly A (SEQ ID NO: 42).

The Envelope plasmid includes a CMV promoter (SEQ ID NO: 29); a beta globin intron (SEQ ID NO: 7 or 8); a VSV-G envelope glycoprotein (SEQ ID NO: 30); and a rabbit beta globin poly A (SEQ ID NO: 42).

In one aspect, the therapeutic lentiviral vector expressing PAH includes all of the elements shown in Vector A of FIG. 3. In another aspect, the therapeutic lentiviral vector expressing PAH includes all of the elements shown in Vector B of FIG. 3. In another aspect, the therapeutic lentiviral vector expressing PAH includes all of the elements shown in Vector C of FIG. 3. In another aspect, the therapeutic lentiviral vector expressing PAH includes all of the elements shown in Vector D of FIG. 3. In another aspect, the therapeutic lentiviral vector expressing PAH includes all of the elements shown in Vector E of FIG. 3. In another aspect, the therapeutic lentiviral vector expressing PAH includes all of the elements shown in Vector F of FIG. 3. In another aspect, the therapeutic lentiviral vector expressing PAH includes all of the elements shown in Vector G of FIG. 3. In another aspect, the therapeutic lentiviral vector expressing PAH includes all of the elements shown in Vector H of FIG. 3.

Synthesis of a 4-vector system, which includes a 3-vector lentiviral packaging system consisting of Helper, Rev, and Envelope plasmids, is disclosed.

Materials and Methods:

Construction of the Helper Plasmid without Rev:

The Helper plasmid without Rev was constructed by inserting a DNA fragment containing the RRE and rabbit beta globin poly A sequence. This sequence was synthesized by Eurofins Genomics with flanking XbaI and XmaI restriction sites. The RRE/rabbit poly A beta globin sequence was then inserted into the Helper plasmid at the XbaI and XmaI restriction sites.

The DNA sequence is as follows:

(SEQ ID NO: 46)
TCTAGAATGGCAGGAAGAAGCGGAGACAGCGACGAAGAGCTCATCAGA
ACAGTCAGACTCATCAAGCTTCTCTATCAAAGCAACCCACCTCCCAAT
CCCGAGGGGACCCGACAGGCCCGAAGGAATAGAAGAAGAAGGTGGAGA
GAGAGACAGAGACAGATCCATTCGATTAGTGAACGGATCCTTGGCACT
TATCTGGGACGATCTGCGGAGCCTGTGCCTCTTCAGCTACCACCGCTT
GAGAGACTTACTCTTGATTGTAACGAGGATTGTGGAACTTCTGGGACG
CAGGGGGTGGGAAGCCCTCAAATATTGGTGGAATCTCCTACAATATTG
GAGTCAGGAGCTAAAGAATAGAGGAGCTTTGTTCCTTGGGTTCTTGGG
AGCAGCAGGAAGCACTATGGGCGCAGCGTCAATGACGCTGACGGTACA
GGCCAGACAATTATTGTCTGGTATAGTGCAGCAGCAGAACAATTTGCT
GAGGGCTATTGAGGCGCAACAGCATCTGTTGCAACTCACAGTCTGGGG
CATCAAGCAGCTCCAGGCAAGAATCCTGGCTGTGGAAAGATACCTAAA
GGATCAACAGCTCCTAGATCTTTTTCCCTCTGCCAAAAATTATGGGGA
CATCATGAAGCCCCTTGAGCATCTGACTTCTGGCTAATAAAGGAAATT
TATTTTCATTGCAATAGTGTGTTGGAATTTTTTGTGTCTCTCACTCGG
AAGGACATATGGGAGGGCAAATCATTTAAAACATCAGAATGAGTATTT
GGTTTAGAGTTTGGCAACATATGCCATATGCTGGCTGCCATGAACAAA
GGTGGCTATAAAGAGGTCATCAGTATATGAAACAGCCCCCTGCTGTCC
ATTCCTTATTCCATAGAAAAGCCTTGACTTGAGGTTAGATTTTTTTTA
TATTTTGTTTTGTGTTATTTTTTTCTTTAACATCCCTAAAATTTTCCT
TACATGTTTTACTAGCCAGATTTTTCCTCCTCTCCTGACTACTCCCAG
TCATAGCTGTCCCTCTTCTCTTATGAAGATCCCTCGACCTGCAGCCCA
AGCTTGGCGTAATCATGGTCATAGCTGTTTCCTGTGTGAAATTGTTAT
CCGCTCACAATTCCACACAACATACGAGCCGGAAGCATAAAGTGTAAA
GCCTGGGGTGCCTAATGAGTGAGCTAACTCACATTAATTGCGTTGCGC
TCACTGCCCGCTTTCCAGTCGGGAAACCTGTCGTGCCAGCGGATCCGC
ATCTCAATTAGTCAGCAACCATAGTCCCGCCCCTAACTCCGCCCATCC
CGCCCCTAACTCCGCCCAGTTCCGCCCATTCTCCGCCCCATGGCTGAC
TAATTTTTTTTATTTATGCAGAGGCCGAGGCCGCCTCGGCCTCTGAGC
TATTCCAGAAGTAGTGAGGAGGCTTTTTTGGAGGCCTAGGCTTTTGCA
AAAAGCTAACTTGTTTATTGCAGCTTATAATGGTTACAAATAAAGCAA
TAGCATCACAAATTTCACAAATAAAGCATTTTTTTCACTGCATTCTAG
TTGTGGTTTGTCCAAACTCATCAATGTATCTTATCAGCGGCCGCCCCG
GG

Construction of the Rev Plasmid:

The RSV promoter and HIV Rev sequences were synthesized as a single DNA fragment by Eurofins Genomics with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter. The DNA sequence was as follows:

(SEQ ID NO: 48)
CAATTGCGATGTACGGGCCAGATATACGCGTATCTGAGGGGACTAGGG
TGTGTTTAGGCGAAAAGCGGGGCTTCGGTTGTACGCGGTTAGGAGTCC
CCTCAGGATATAGTAGTTTCGCTTTTGCATAGGGAGGGGGAAATGTAG
TCTTATGCAATACACTTGTAGTCTTGCAACATGGTAACGATGAGTTAG
CAACATGCCTTACAAGGAGAGAAAAAGCACCGTGCATGCCGATTGGTG
GAAGTAAGGTGGTACGATCGTGCCTTATTAGGAAGGCAACAGACAGGT
CTGACATGGATTGGACGAACCACTGAATTCCGCATTGCAGAGATAATT
GTATTTAAGTGCCTAGCTCGATACAATAAACGCCATTTGACCATTCAC
CACATTGGTGTGCACCTCCAAGCTCGAGCTCGTTTAGTGAACCGTCAG
ATCGCCTGGAGACGCCATCCACGCTGTTTTGACCTCCATAGAAGACAC
CGGGACCGATCCAGCCTCCCCTCGAAGCTAGCGATTAGGCATCTCCTA
TGGCAGGAAGAAGCGGAGACAGCGACGAAGAACTCCTCAAGGCAGTCA
GACTCATCAAGTTTCTCTATCAAAGCAACCCACCTCCCAATCCCGAGG
GGACCCGACAGGCCCGAAGGAATAGAAGAAGAAGGTGGAGAGAGAGAC
AGAGACAGATCCATTCGATTAGTGAACGGATCCTTAGCACTTATCTGG
GACGATCTGCGGAGCCTGTGCCTCTTCAGCTACCACCGCTTGAGAGAC
TTACTCTTGATTGTAACGAGGATTGTGGAACTTCTGGGACGCAGGGGG
TGGGAAGCCCTCAAATATTGGTGGAATCTCCTACAATATTGGAGTCAG
GAGCTAAAGAATAGTCTAGA

The plasmids used in the packaging systems can be modified with similar elements, and the intron sequences can potentially be removed without loss of vector function. For example, the following elements can replace similar elements in the packaging system:

Promoters: Elongation Factor-1 (EF-1) (SEQ ID NO: 49), phosphoglycerate kinase (PGK) (SEQ ID NO: 50), and ubiquitin C (UbC) (SEQ ID NO: 51) can replace the CMV (SEQ ID NO: 29) or CMV enhancer/chicken beta actin promoter (SEQ ID NO: 23). These sequences can also be further varied by addition, substitution, deletion or mutation.

Poly A sequences: SV40 poly A (SEQ ID NO: 52) and bGH poly A (SEQ ID NO: 53) can replace the rabbit beta globin poly A (SEQ ID NO: 42). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (SEQ ID NO: 24); HIV Pol (SEQ ID NO: 25); and HIV Int (SEQ ID NO: 26) from the Bal strain can be interchanged with the gag, pol, and int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) (SEQ ID NO: 54), gibbon ape leukemia virus (GALV) (SEQ ID NO: 55), Rabies (FUG) (SEQ ID NO: 56), lymphocytic choriomeningitis virus (LCMV) (SEQ ID NO: 57), influenza A fowl plague virus (FPV) (SEQ ID NO: 58), Ross River alphavirus (RRV) (SEQ ID NO: 59), murine leukemia virus 10A1 (MLV) (SEQ ID NO: 60), or Ebola virus (EboV) (SEQ ID NO: 61). Sequences for these envelopes are identified in the sequence portion herein. Further, these sequences can also be further varied by addition, substitution, deletion or mutation.

In summary, the 3-vector versus 4-vector systems can be compared and contrasted as follows. The 3-vector lentiviral vector system contains: 1. Helper plasmid: HIV Gag, Pol, Integrase, RRE, and Rev; 2. Envelope plasmid: VSV-G envelope; and 3. Therapeutic vector: RSV, 5′LTR, Psi Packaging Signal, RRE, cPPT, prothrombin enhancer, alpha 1 anti-trypsin promoter, phenylalanine hydroxylase, WPRE, and 3′delta LTR. The 4-vector lentiviral vector system contains: 1. Helper plasmid: HIV Gag, Pol, Integrase, and RRE; 2. Rev plasmid: Rev; 3. Envelope plasmid: VSV-G envelope; and 4. Therapeutic vector: RSV, 5′LTR, Psi Packaging Signal, RRE, cPPT, prothrombin enhancer, alpha 1 anti-trypsin promoter, phenylalanine hydroxylase, WPRE, and 3′delta LTR. Sequences corresponding with the above elements are identified in the sequence listings portion herein.

### Example 2. Therapeutic Vectors

Exemplary therapeutic vectors have been designed and developed as shown, for example, in FIG. 3.

Referring first to Vector A of FIG. 3, from left to right, the key genetic elements are as follows: hybrid 5′ long terminal repeat (RSV/LTR), Psi sequence (RNA packaging site), RRE (Rev-response element), cPPT (polypurine tract), a prothrombin enhancer, a hAAT promoter, a PAH sequence including the PAH sequences and variants thereof, as detailed herein, Woodchuck Post-Transcriptional Regulatory Element (WPRE), and LTR with a deletion in the U3 region.

Referring next to Vector B of FIG. 3, from left to right, the key genetic elements are as follows: hybrid 5′ long terminal repeat (RSV/LTR), Psi sequence (RNA packaging site), RRE (Rev-response element), cPPT (polypurine tract), a HNF1 (hepatocyte nuclear factor) binding site upstream of a prothrombin enhancer, a hAAT promoter, a PAH sequence including the PAH sequences and variants thereof, as detailed herein, a Woodchuck Post-Transcriptional Regulatory Element (WPRE), and LTR with a deletion in the U3 region.

Referring next to Vector C of FIG. 3, from left to right, the key genetic elements are as follows: hybrid 5′ long terminal repeat (RSV/LTR), Psi sequence (RNA packaging site), RRE (Rev-response element), cPPT (polypurine tract), a HNF1/4 (hepatocyte nuclear factor) binding site upstream of a prothrombin enhancer, a hAAT promoter, a PAH sequence including the PAH sequences and variants thereof, as detailed herein, a Woodchuck Post-Transcriptional Regulatory Element (WPRE), and LTR with a deletion in the U3 region.

Referring next to Vector D of FIG. 3, from left to right, the key genetic elements are as follows: hybrid 5′ long terminal repeat (RSV/LTR), Psi sequence (RNA packaging site), RRE (Rev-response element), cPPT (polypurine tract), a prothrombin enhancer, a HNF1 (hepatocyte nuclear factor), a hAAT promoter, a PAH sequence including the PAH sequences and variants thereof, as detailed herein, a Woodchuck Post-Transcriptional Regulatory Element (WPRE), and LTR with a deletion in the U3 region.

Referring next to Vector E of FIG. 3, from left to right, the key genetic elements are as follows: hybrid 5′ long terminal repeat (RSV/LTR), Psi sequence (RNA packaging site), RRE (Rev-response element), cPPT (polypurine tract), a prothrombin enhancer, a HNF1/4 (hepatocyte nuclear factor), a hAAT promoter, a PAH sequence including the PAH sequences and variants thereof as detailed herein, a Woodchuck Post-Transcriptional Regulatory Element (WPRE), and LTR with a deletion in the U3 region.

Referring next to Vector F of FIG. 3, from left to right, the key genetic elements are as follows: hybrid 5′ long terminal repeat (RSV/LTR), Psi sequence (RNA packaging site), RRE (Rev-response element), cPPT (polypurine tract), five HNF1 (hepatocyte nuclear factor) binding sites upstream of a prothrombin enhancer, a hAAT promoter, a PAH sequence including the PAH sequences and variants thereof, as detailed herein, a Woodchuck Post-Transcriptional Regulatory Element (WPRE), and LTR with a deletion in the U3 region.

Referring next to Vector G of FIG. 3, from left to right, the key genetic elements are as follows: hybrid 5′ long terminal repeat (RSV/LTR), Psi sequence (RNA packaging site), RRE (Rev-response element), cPPT (polypurine tract), three HNF/HNF4 (hepatocyte nuclear factor) binding sites upstream of a prothrombin enhancer, a hAAT promoter, a PAH sequence including the PAH sequences and variants thereof, as detailed herein, a Woodchuck Post-Transcriptional Regulatory Element (WPRE), and LTR with a deletion in the U3 region.

Referring first to Vector H of FIG. 3, from left to right, the key genetic elements are as follows: hybrid 5′ long terminal repeat (RSV/LTR), Psi sequence (RNA packaging site), RRE (Rev-response element), cPPT (polypurine tract), a prothrombin enhancer, a hAAT promoter, a rabbit beta globin intron, a PAH sequence including the PAH sequences and variants thereof, as detailed herein, Woodchuck Post-Transcriptional Regulatory Element (WPRE), and LTR with a deletion in the U3 region.

To produce the vectors outlined generally in FIG. 3, the methods and materials described herein and as otherwise as understood by those skilled in the art were employed.

Inhibitory RNA Design:

The sequence of Homo sapiens phenylalanine hydroxylase (PAH) (NM_000277.1) mRNA was used to search for potential shRNA candidates to knockdown PAH levels in human cells. Potential RNA shRNA sequences were chosen from candidates selected by siRNA or shRNA design programs such as from the GPP Web Portal hosted by the Broad Institute (portals.broadinstitute.org/gpp/public/) or the BLOCK-iT RNAi Designer from Thermo Scientific (https://rnaidesigner.thermofisher.com/rnaiexpress/). Individual selected shRNA sequences were inserted into a lentiviral vector immediately 3 prime to a RNA polymerase III promoter H1 (SEQ ID NO: 22) to regulate shRNA expression. These lentivirus shRNA constructs were used to transduce cells and measure the change in specific mRNA levels.

Vector Construction:

For PAH shRNA, oligonucleotide sequences containing BamHI and EcoRI restriction sites were synthesized by Eurofins MWG Operon. Overlapping sense and antisense oligonucleotide sequences were mixed and annealed during cooling from 70 degrees Celsius to room temperature. The lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Thermo Scientific. The DNA concentrations were determined and vector to oligo (3:1 ratio) were mixed, allowed to anneal, and ligated. The ligation reaction was performed with T4 DNA ligase for 30 minutes at room temperature. 2.5 microliters of the ligation mix were added to 25 microliters of STBL3 competent bacterial cells. Transformation was achieved after heat-shock at 42 degrees Celsius. Bacterial cells were spread on agar plates containing ampicillin and drug-resistant colonies (indicating the presence of ampicillin-resistance plasmids) were recovered and expanded in LB broth. To check for insertion of the oligo sequences, plasmid DNA was extracted from harvested bacteria cultures with the Thermo Scientific DNA mini prep kit. Insertion of shRNA sequences in the lentiviral vector was verified by DNA sequencing using a specific primer for the promoter used to regulate shRNA expression. Using the following target sequences, exemplary shRNA sequences were determined to knock-down PA.

PAH shRNA sequence #1:
(SEQ ID NO: 13)
TCGCATTTCATCAAGATTAATCTCGAGATTAATCTTGATGAAATGC
GATTTTT
PAH shRNA sequence #2:
(SEQ ID NO: 14)
ACTCATAAAGGAGCATATAAGCTCGAGCTTATATGCTCCTTTATGA
GTTTTTT

### Example 3. Liver Specific Prothrombin Enhancer/hAAT Promoter

Hepa1-6 mouse hepatoma cells were transduced with lentiviral vectors containing a liver-specific prothrombin enhancer (SEQ ID NO: 5), and a human alpha-1 anti-trypsin promoter (SEQ ID NO: 6). The resulting DNA sequence is as follows:

(SEQ ID NO: 63)
GCGAGAACTTGTGCCTCCCCGTGTTCCTGCTCTTTGTCCCTCTGTCCT
ACTTAGACTAATATTTGCCTTGGGTACTGCAAACAGGAAATGGGGGAG
GGACAGGAGTAGGGCGGAGGGTAGCCCGGGGATCTTGCTACCAGTGGA
ACAGCCACTAAGGATTCTGCAGTGAGAGCAGAGGGCCAGCTAAGTGGT
ACTCTCCCAGAGACTGTCTGACTCACGCCACCCCCTCCACCTTGGACA
CAGGACGCTGTGGTTTCTGAGCCAGGTACAATGACTCCTTTCGGTAAG
TGCAGTGGAAGCTGTACACTGCCCAGGCAAAGCGTCCGGGCAGCGTAG
GCGGGCGACTCAGATCCCAGCCAGTGGACTTAGCCCCTGTTTGCTCCT
CCGATAACTGGGGTGACCTTGGTTAATATTCACCAGCAGCCTCCCCCG
TTGCCCCTCTGGATCCACTGCTTAAATACGGACGAGGACAGGGCCCTG
TCTCCTCAGCTTCAGGCACCACCACTGACCTGGGACAGTGAAT.

Results for these infections are detailed in further Examples herein.

### Example 4. hAAT Promoter with Prothrombin Enhancer and Hepatocyte Nuclear Factor (HNF) Binding Sites

Hepa1-6 mouse hepatoma cells were transduced with lentiviral vectors containing a liver-specific prothrombin enhancer (SEQ ID NO: 5), a human alpha-1 anti-trypsin promoter (SEQ ID NO: 6), and one or more hepatocyte nuclear factor (HNF) binding sites. The resulting DNA sequence that includes five HNF1 binding sites (designated in underlined font) was as follows:

(SEQ ID NO: 64)
GTTAATCATTAACGTTAATCATTAACGTTAATCATTAACGTTAATCAT
TAACGTTAATCATTAACATCGATGCGAGAACTTGTGCCTCCCCGTGTT
CCTGCTCTTTGTCCCTCTGTCCTACTTAGACTAATATTTGCCTTGGGT
ACTGCAAACAGGAAATGGGGGAGGGACAGGAGTAGGGCGGAGGGTAGG
ATTCTGCAGTGAGAGCAGAGGGCCAGCTAAGTGGTACTCTCCCAGAGA
CTGTCTGACTCACGCCACCCCCTCCACCTTGGACACAGGACGCTGTGG
TTTCTGAGCCAGGTACAATGACTCCTTTCGGTAAGTGCAGTGGAAGCT
GTACACTGCCCAGGCAAAGCGTCCGGGCAGCGTAGGCGGGCGACTCAG
ATCCCAGCCAGTGGACTTAGCCCCTGTTTGCTCCTCCGATAACTGGGG
TGACCTTGGTTAATATTCACCAGCAGCCTCCCCCGTTGCCCCTCTGGA
TCCACTGCTTAAATACGGACGAGGACAGGGCCCTGTCTCCTCAGCTTC
AGGCACCACCACTGACCTGGGACAGTGAAT.

The resulting DNA sequence that includes three HNF1/HNF4 binding sites (HNF1 designated in underlined font; HNF4 designated in bold font) is as follows:

(SEQ ID NO: 65)
GTTAATCATTAACGCTTGTACTTTGGTACAGTTAATCATTAACGCTTG
TACTTTGGTACAGTTAATCATTAACGCTTGTACTTTGGTACAATCGAT
GCGAGAACTTGTGCCTCCCCGTGTTCCTGCTCTTTGTCCCTCTGTCCT
ACTTAGACTAATATTTGCCTTGGGTACTGCAAACAGGAAATGGGGGAG
GGACAGGAGTAGGGCGGAGGGTAGCCCGGGGATTCTGCAGTGAGAGCA
GAGGGCCAGCTAAGTGGTACTCTCCCAGAGACTGTCTGACTCACGCCA
CCCCCTCCACCTTGGACACAGGACGCTGTGGTTTCTGAGCCAGGTACA
ATGACTCCTTTCGGTAAGTGCAGTGGAAGCTGTACACTGCCCAGGCAA
AGCGTCCGGGCAGCGTAGGCGGGCGACTCAGATCCCAGCCAGTGGACT
TAGCCCCTGTTTGCTCCTCCGATAACTGGGGTGACCTTGGTTAATATT
CACCAGCAGCCTCCCCCGTTGCCCCTCTGGATCCACTGCTTAAATACG
GACGAGGACAGGGCCCTGTCTCCTCAGCTTCAGGCACCACCACTGACC
TGGGACAGTGAAT.

The expression of PAH from these vectors is detailed in further Examples herein.

### Example 5. Materials and Methods for PAH

The sequence of Homo sapiens phenylalanine hydroxylase (hPAH) mRNA (Gen Bank: NM_000277.1) was chemically synthesized with EcoRI and SalI restriction enzyme sites located at distal and proximal ends of the gene by Eurofins Genomics (Louisville, Ky.). hPAH treated with EcoRI and SalI restriction enzymes was ligated into the pCDH lentiviral plasmids (System Biosciences, Palo Alto, Calif.) under control of a hybrid promoter comprising parts of ApoE (NM_000001.11, U35114.1) or prothrombin (AF478696.1) and hAAT (HG98385.1) locus control regions. Additionally, human PAH was synthesized to include 289 nucleotides of the 3′ untranslated region (UTR).

The lentiviral vector and hPAH sequences were digested with the restriction enzymes BamHI and EcoRI (NEB, Ipswich, Mass.) for two hours at 37 degrees Celsius. The digested lentiviral vector was purified by agarose gel electrophoresis and extracted from the gel using a DNA gel extraction kit from ThermoFisher (Waltham, Mass.). The DNA concentration was determined and then mixed with the PAH sequence (hPAH) using an insert to vector ratio of 3:1. The mixture was ligated with T4 DNA ligase (NEB) for 30 minutes at room temperature. 2.5 microliters of the ligation mix were added to 25 microliters of STBL3 competent bacterial cells (ThermoFisher). Transformation was carried out by heat-shock at 42 degrees Celsius. Bacterial cells were streaked onto agar plates containing ampicillin and then colonies were expanded in LB broth. To check for insertion of the PAH sequences, Plasmid DNA was extracted from harvested bacteria cultures with the ThermoFisher DNA mini prep kit. Insertion of the PAH sequence in the lentiviral vector (LV) was verified by DNA sequencing. Next, the ApoE enhancer/hAAT promoter or prothrombin enhancer/hAAT promoter sequences with ClaI and EcoRI restriction sites were synthesized by Eurofins Genomics. The lentiviral vector containing a PAH coding sequence and the hybrid promoters were digested with ClaI and EcoRI enzymes and ligated together. The plasmids containing the hybrid promoters were verified by DNA sequencing. The lentiviral vector containing hPAH and a hybrid promoter sequence were then used to package lentiviral particles to test for their ability to express PAH in transduced cells. Mammalian cells were transduced with lentiviral particles. Cells were collected after 3 days and protein was analyzed by immunoblot for PAH expression.

Modifications of the hPAH Sequence:

Several modifications of the hPAH sequence were incorporated to improve cellular expression levels as regulated by the ApoE enhancer/hAAT promoter. First, 289 nucleotides of the hPAH 3′ untranslated region (UTR) was inserted after the PAH coding region and before the mRNA terminus. This created LV-ApoE/hAAT-hPAH-UTR

Next, liver-specific ApoE enhancer was exchanged with the liver-specific prothrombin enhancer. The expression of PAH was analyzed with either the ApoE or prothrombin enhancer/hAAT promoter combination with the hPAH coding sequence and 289 nucleotides of the UTR. Next, the expression of PAH was assessed with the prothrombin enhancer/hAAT promoter and hPAH coding sequence without the UTR region. The combination of the prothrombin enhancer/hAAT promoter obviated the requirement of the UTR region as was required with the ApoE enhancer/hAAT promoter combination. Therefore, the prothrombin enhancer/hAAT promoter combination can regulate high levels of PAH expression in a liver-specific manner without the requirement of the UTR region. This important advance in understanding liver-specific regulatory elements to regulate the hPAH gene allows for the generation of constructs for specific expression in liver tissue while still achieving high-level production of hPAH. Restricting transgene expression to liver cells is an important consideration for vector safety and target specificity in a genetic medicine for phenylketonuria.

### Example 6. Lentivirus-Delivered Expression of hPAH with Variations of the Prothrombin Enhancer and with or without the 3′ UTR Region in Hepa1-6 Cells and 293T Cells

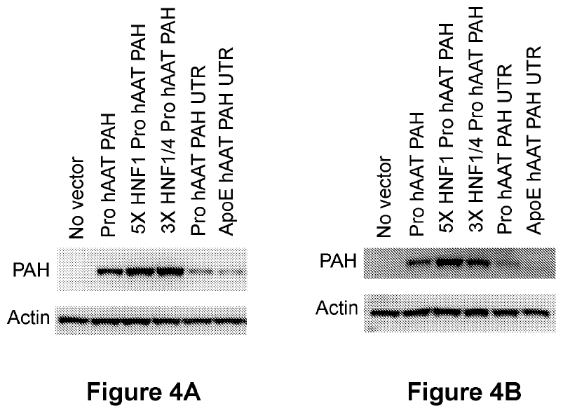

This Example illustrates that expression of human PAH is increased in Hepa1-6 carcinoma cells and 293T human embryonic kidney cells with a lentiviral vector containing the hAAT promoter in combination with the prothrombin enhancer as compared to the ApoE enhancer as shown in FIGS. 4A and 4B, respectively. The 3′ UTR is not required for hPAH expression when the prothrombin enhancer is combined with the hAAT promoter. The 3′ UTR actually decreases PAH expression in the prothrombin containing vector as shown in FIGS. 4A and 4B, respectively. This Example also illustrates that a lentivirus vector expressing Hepatocyte Nuclear Factor 1 and 4 (HNF1/4) binding sites in combination with the prothrombin enhancer increases the levels of PAH protein in Hepa1-6 cells and 293T cells as shown in FIGS. 4A and 4B, respectively.

Human PAH, the prothrombin and ApoE enhancer, and hAAT promoter were synthesized by Eurofins Genomics (Louisville, Ky.) and inserted into a lentiviral vector. Insertion of the sequences was verified by DNA sequencing. The lentiviral vectors containing a verified hPAH sequence were then used to transduce Hepa1-6 mouse liver cancer cells or 293T human embryonic kidney cells (American Type Culture Collection, Manassas, Va.). The lentiviral vectors incorporated a human PAH gene with or without its 3′ UTR. In addition, hPAH expression in these constructs was driven by the hAAT promoter containing either the liver specific prothrombin or ApoE enhancer. Cells were transduced with lentiviral particles and after 3 days protein was analyzed by immunoblot for hPAH expression. The relative expression of human PAH was detected by immunoblot using an anti-PAH antibody (Abcam, Cambridge, Mass.) and an anti-beta actin antibody (SigmaMillipore, Billerica, Mass.) was used for a loading control.

As shown in FIGS. 4A and 4B, six groups are compared: a control comprising Hepa 1-6 cells or 293T cells alone (lane 1), a lentiviral vector expressing the coding region of hPAH by the prothrombin enhancer/hAAT promoter (lane 2), a lentiviral vector expressing hPAH by the prothrombin enhancer/hAAT promoter containing 5×HNF1 binding sites upstream of the prothrombin enhancer (lane 3), a lentiviral vector expressing hPAH by the prothrombin enhancer/hAAT promoter containing 3×HNF1 and 3×HNF4 binding sites upstream of the prothrombin enhancer (lane 4), a lentiviral vector expressing hPAH with the 3′ UTR by the prothrombin enhancer/hAAT promoter (lane 5), and a lentiviral vector expressing hPAH with the 3′ UTR by the ApoE enhancer/hAAT promoter (lane 6). FIGS. 4A and 4B demonstrate that expression of PAH is increased in both Hepa1-6 carcinoma cells and 293T cells when the prothrombin enhancer is combined with the hAAT promoter as compared with the ApoE enhancer. Additionally, the PAH 3′ UTR is not required for hPAH expression when the prothrombin enhancer is included in the vector.

### Example 7. Lentivirus-Delivered Expression of hPAH with an Intron Sequence a Codon Optimized PAH Sequence, and the Prothrombin Enhancer Containing HNF-1 or HNF1/4 Binding Sites in Hepa1-6 Cells

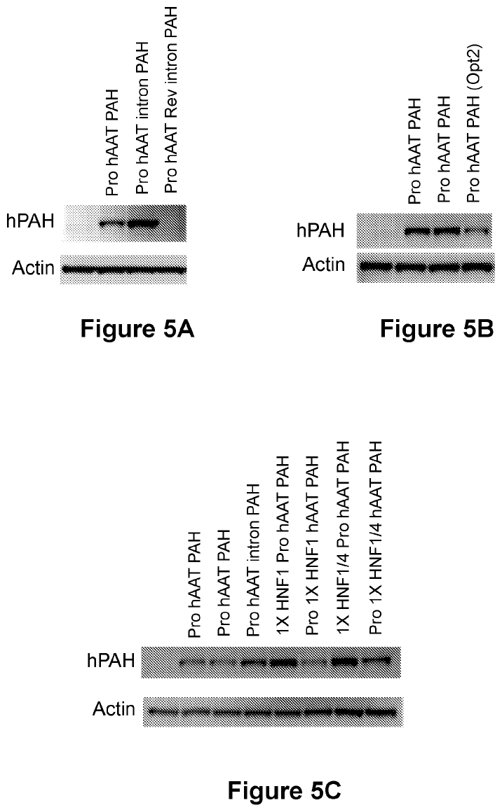

This Example illustrates that expression of human PAH is increased in Hepa1-6 carcinoma cells with a lentiviral vector containing the hAAT promoter in combination with the prothrombin enhancer and a rabbit beta globin intron sequence as shown in FIG. 5A. hPAH is not expressed when the intron sequence is inserted in the reverse direction. In FIG. 5B, this shows that a codon-optimized version of the hPAH coding sequence expresses less than the non-optimized hPAH coding region sequence. This Example also illustrates that a lentiviral vector expressing Hepatocyte Nuclear Factor-1 and -4 (HNF1 and HNF1/4) binding sites in combination with the prothrombin enhancer increases the levels of hPAH protein in Hepa1-6 cells as shown in FIG. 5C.

Human PAH (optimized and non-optimized), the prothrombin enhancer, hAAT promoter, and a rabbit beta globin sequence were synthesized by Eurofins Genomics (Louisville, Ky.) and inserted into a lentiviral vector. Insertion of the sequences was verified by DNA sequencing. The lentiviral vectors containing a verified hPAH sequence were then used to transduce Hepa1-6 mouse liver cancer cells (American Type Culture Collection, Manassas, Va.). The lentiviral vectors incorporated a human PAH gene with or without a rabbit beta globin intron. In addition, hPAH expression in these constructs was driven by the hAAT promoter containing the liver-specific prothrombin enhancer with HNF1 or HNF1/4 binding sites, either upstream or downstream of the prothrombin enhancer. Cells were transduced with lentiviral particles and after 3 days protein was analyzed by immunoblot for PAH expression. The relative expression of human PAH was detected by immunoblot using an anti-PAH antibody (Abcam, Cambridge, Mass.) and an anti-beta actin antibody (SigmaMillipore, Billerica, Mass.) was used for a loading control.

As shown in FIG. 5A, four groups are compared: a control comprising Hepa 1-6 cells alone (lane 1), a lentiviral vector expressing the coding region of hPAH by the prothrombin enhancer/hAAT promoter (lane 2), a lentiviral vector expressing hPAH by the prothrombin enhancer/hAAT promoter and containing a rabbit beta globin intron in the forward direction (lane 3), and a lentiviral vector expressing hPAH by the prothrombin enhancer/hAAT promoter and containing a rabbit beta globin intron in the reverse direction (lane 4). As shown in FIG. 5B, three groups are compared: a control comprising Hepa 1-6 cells alone (lane 1), a lentiviral vector expressing only the coding region of hPAH by the prothrombin enhancer/hAAT promoter (lane 2 and 3), and a lentiviral vector expressing a codon-optimized sequence of hPAH by the prothrombin enhancer/hAAT promoter (lane 4). As shown in FIG. 5C, six groups are compared: a control comprising Hepa 1-6 cells alone (lane 1), a lentiviral vector only expressing the coding region of hPAH by the prothrombin enhancer/hAAT promoter (lane 2 and 3), a lentiviral vector expressing hPAH by the prothrombin enhancer/hAAT promoter and containing a rabbit beta globin intron (lane 4), a lentiviral vector expressing hPAH by the prothrombin enhancer/hAAT promoter and a HNF1 binding site upstream of the prothrombin enhancer (lane 5), a lentiviral vector expressing hPAH by the prothrombin enhancer/hAAT promoter and a HNF1 binding site downstream of the prothrombin enhancer (lane 6), a lentiviral vector expressing hPAH by the prothrombin enhancer/hAAT promoter and a HNF1/4 binding site upstream of the prothrombin enhancer (lane 7), and a lentiviral vector expressing hPAH by the prothrombin enhancer/hAAT promoter and a HNF1/4 binding site downstream of the prothrombin enhancer (lane 8). FIGS. 5A-5C demonstrate that expression of hPAH is increased in Hepa1-6 carcinoma cells when a rabbit beta globin intron sequence is added upstream of the hPAH coding sequence. Additionally, a codon-optimized hPAH coding sequence expresses less than a non-optimized sequence. Finally, the addition of HNF1 or HNF1/4 binding sites upstream, but not downstream, of the prothrombin enhancer increases the expression of hPAH as compared with the lentiviral vector containing only the prothrombin enhancer/hAAT promoter.

### Example 8. Lentivirus-Delivered Expression of hPAH RNA with the hAAT Promoter and Prothrombin Enhancer Containing HNF-1 and HNF-1/4 Binding Sites in Hepa1-6 Cells

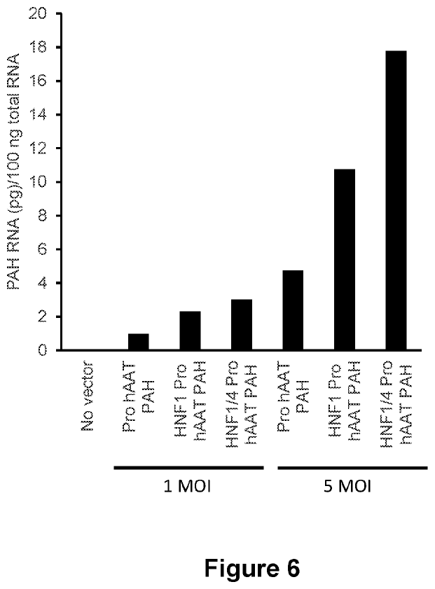

As shown in FIG. 6, this Example illustrates that expression of human PAH RNA is increased in Hepa1-6 carcinoma cells transduced at a multiplicity of infection (MOI) of 1 and 5 with a lentiviral vector containing the hAAT promoter in combination with the prothrombin enhancer and binding sites for HNF1 and HNF1/4.

Human PAH, the prothrombin enhancer, and hAAT promoter were synthesized by Eurofins Genomics (Louisville, Ky.) and inserted into a lentiviral vector. Insertion of the sequences was verified by DNA sequencing. The lentiviral vectors containing a verified hPAH sequence were then used to transduce Hepa1-6 mouse liver cancer cells (American Type Culture Collection, Manassas, Va.). The lentiviral vectors incorporated a human PAH gene. In addition, hPAH expression in these constructs was driven by the hAAT promoter containing the liver-specific prothrombin enhancer with upstream HNF1 or HNF1/4 binding sites. Cells were transduced with lentiviral particles and after 3 days RNA was extracted with the RNeasy kit (Qiagen, Germantown, Md.) and analyzed by qPCR analysis with a TaqMan probe (5′-TCGTGAAAGCTCATGGACAGTGGC-3′) (SEQ ID NO: 66) and primer set Fwd: 5′-AGATCTTGAGGCATGACATTGG-3′ (SEQ ID NO: 67) and Rev: 5′-GTCCAGCTCTGAATGGTCTT-3′ (SEQ ID NO: 68) for hPAH. Total RNA (100 ng) was normalized with an actin probe (5′-AGCGGGAAATCGTGCGTGAC-3′) (SEQ ID NO: 69) and primer set (Fwd: 5′-GGACCTGACTGACTACCTCAT-3′ (SEQ ID NO: 70) and Rev: 5′-CGTAGCACAGCTTCTCCTTAAT-3′) (SEQ ID NO: 71).

As shown in FIG. 6, four groups are compared: a control comprising Hepa 1-6 cells alone (bar 1), a lentiviral vector expressing the coding region of hPAH by the prothrombin enhancer/hAAT promoter at 1 MOI (bar 2) and 5 MOI (bar 5), a lentiviral vector expressing hPAH by the prothrombin enhancer/hAAT promoter and a HNF1 binding site upstream of the prothrombin enhancer at 1 MOI (bar 3) and 5 MOI (bar 6), and a lentiviral vector expressing hPAH by the prothrombin enhancer/hAAT promoter and a HNF1/4 binding site upstream of the prothrombin enhancer at 1 MOI (bar 4) and 5 MOI (bar 7). FIG. 6 demonstrates that expression of PAH RNA is increased from 1 to 4.7 pg (1-5 MOI) with a vector expressing hPAH by a prothrombin enhancer/hAAT promoter. When a HNF1 binding site is included upstream of the prothrombin enhancer there is an increase from 2.3 to 10.7 pg (1-5 MOI) and an increase from 3 to 17.8 pg (1-5 MOI) when a HNF1/4 binding site is inserted upstream of the prothrombin enhancer.

### Example 9. Lentivirus-Delivered Expression of hPAH with the Prothrombin Enhancer and Either the hAAT or Thyroxin Binding Globulin (TBG) Promoter in Hepa1-6 Cells

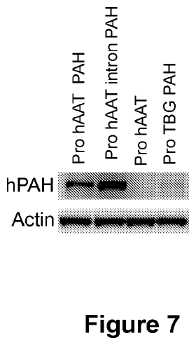

This Example illustrates that expression of human PAH is increased in Hepa1-6 carcinoma cells with a lentiviral vector containing the prothrombin enhancer in combination with the hAAT promoter as compared to the TBG promoter (SEQ ID NO: 62) as shown in FIG. 7.

Human PAH, the prothrombin enhancer, and hAAT and TBG promoter were synthesized by Eurofins Genomics (Louisville, Ky.) and inserted into a lentiviral vector. Insertion of the sequences was verified by DNA sequencing. The lentiviral vectors containing a verified hPAH sequence were then used to transduce Hepa1-6 mouse liver cancer cells (American Type Culture Collection, Manassas, Va.). The lentiviral vectors incorporated a human PAH gene. In addition, hPAH expression in these constructs was driven by either the liver-specific hAAT or TBG promoter. Cells were transduced with lentiviral particles and after 3 days protein was analyzed by immunoblot for PAH expression. The relative expression of human PAH was detected by immunoblot using an anti-PAH antibody (Abcam, Cambridge, Mass.) and an anti-beta actin antibody (SigmaMillipore, Billerica, Mass.) was used for a loading control.

As shown in FIG. 7, four groups are compared: a lentiviral vector expressing the coding region of hPAH by the prothrombin enhancer/hAAT promoter (lane 1), a lentiviral vector expressing hPAH by the prothrombin enhancer/hAAT promoter and containing a rabbit beta globin intron (lane 2), a control with a lentiviral vector containing only the prothrombin enhancer/hAAT promoter (lane 3), and a lentiviral vector expressing hPAH by the prothrombin enhancer/TBG promoter (lane 4). FIG. 7 demonstrates that expression of PAH is substantially increased in Hepa1-6 carcinoma cells when the prothrombin enhancer is combined with the hAAT promoter as compared with the TBG promoter.

### Example 10. Lentivirus-Delivered Expression of hPAH with Either a Rabbit or Human Beta Globin Intron Sequence Upstream of the PAH Gene in Hepa1-6 Cells or Hep3B Cells

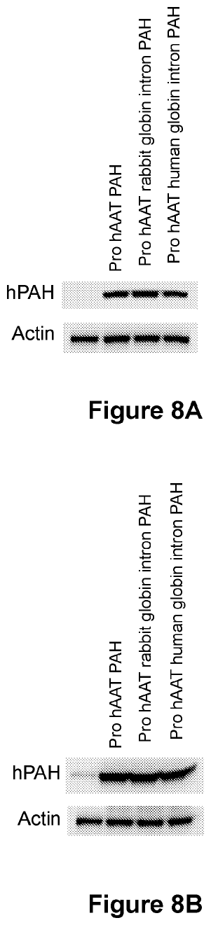

This Example illustrates that expression of human PAH in Hepa1-6 and Hep3B carcinoma cells with a lentiviral vector containing the prothrombin enhancer in combination with the hAAT promoter and either a rabbit or human beta globin intron is not increased with the human beta globin intron as shown in FIGS. 8A and 8B.

Human PAH, the prothrombin enhancer, hAAT promoter, and rabbit or human beta globin intron were synthesized by Eurofins Genomics (Louisville, Ky.) and inserted into a lentiviral vector. Insertion of the sequences was verified by DNA sequencing. The lentiviral vectors containing a verified hPAH sequence were then used to transduce Hepa1-6 mouse liver cancer cells or Hep3B human hepatocellular carcinoma cells (American Type Culture Collection, Manassas, Va.). The lentiviral vectors incorporated a human PAH gene. In addition, hPAH expression in these constructs was driven by the liver-specific hAAT promoter and either a rabbit or human beta globin intron. Cells were transduced with lentiviral particles and after 3 days protein was analyzed by immunoblot for PAH expression. The relative expression of human PAH was detected by immunoblot using an anti-PAH antibody (Abcam, Cambridge, Mass.) and an anti-beta actin antibody (SigmaMillipore, Billerica, Mass.) was used for a loading control.

As shown in FIGS. 8A and 8B, four groups are compared: no lentivirus (lane 1), a lentiviral vector expressing hPAH by the prothrombin enhancer/hAAT promoter (lane 2), a lentiviral vector expressing hPAH by the prothrombin enhancer/hAAT promoter and a rabbit beta globin intron sequence (lane 3), and a lentiviral vector expressing hPAH by the prothrombin enhancer/hAAT promoter and a human beta globin intron (lane 4). FIGS. 8A and 8B demonstrate that expression of PAH is increased in Hepa1-6 and Hep3B carcinoma cells with the prothrombin enhancer and hAAT promoter. Addition of the rabbit beta globin intron improves expression in Hepa1-6 cells but not Hep3B cells. The human beta globin intron does not improve expression in either Hepa1-6 or Hep3B cells.

### Example 11. Lentivirus-Delivered Expression of hPAH with the Prothrombin Enhancer/hAAT Promoter in Primary Human Hepatocytes

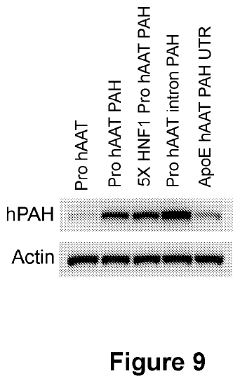

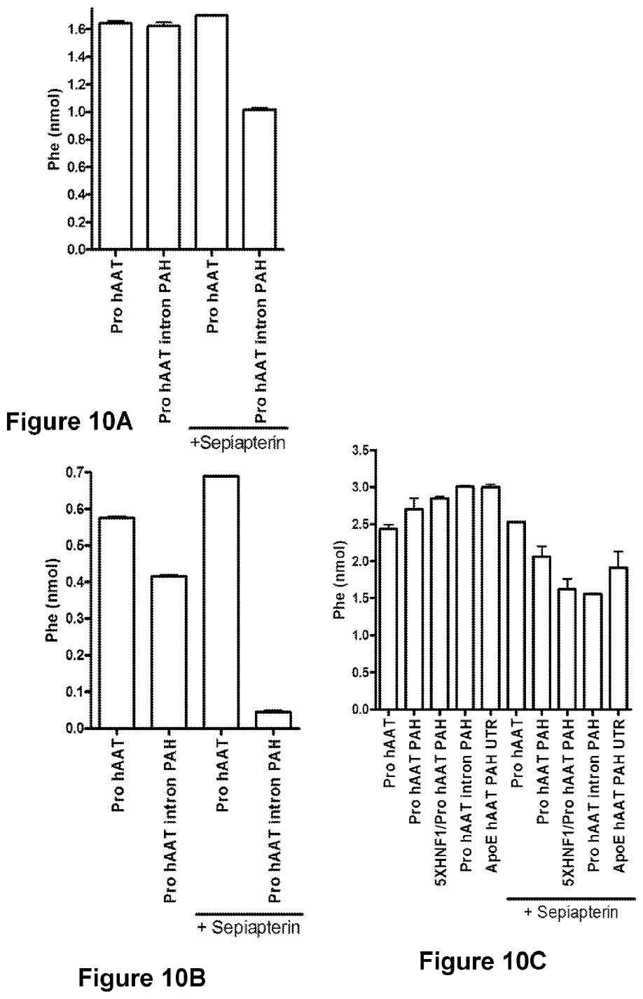

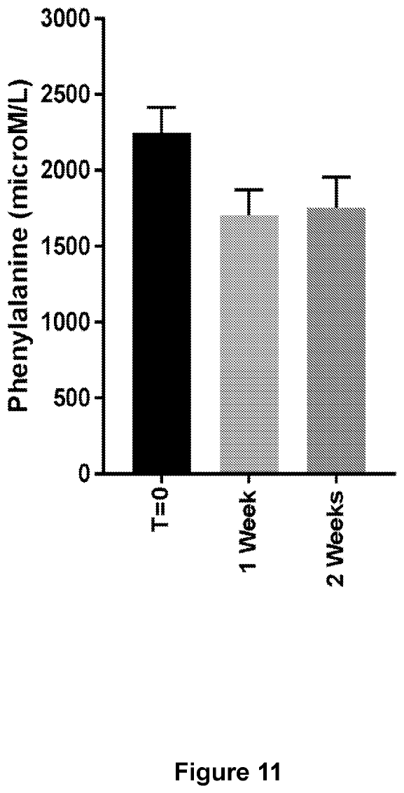

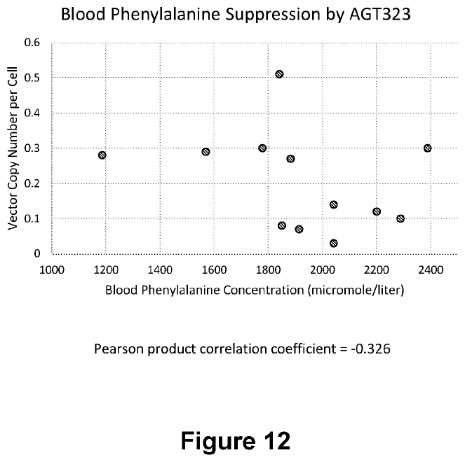

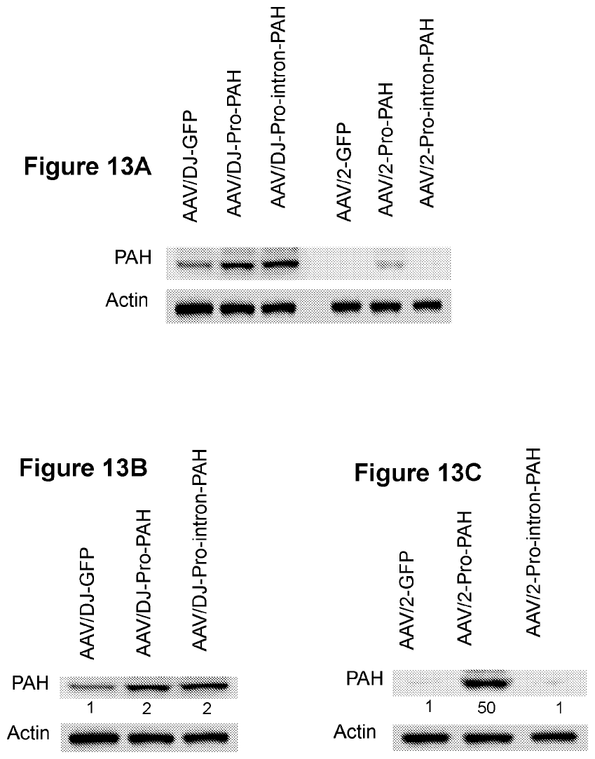

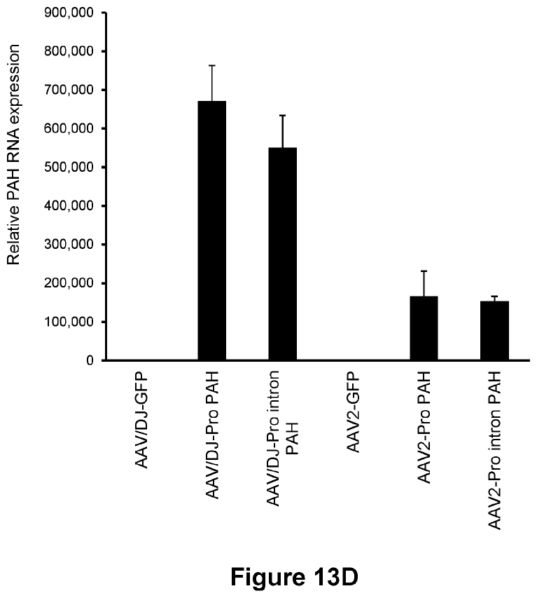

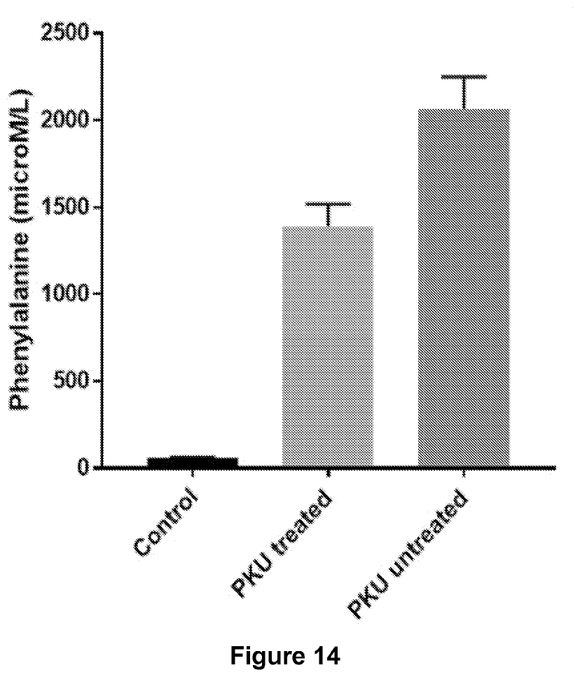

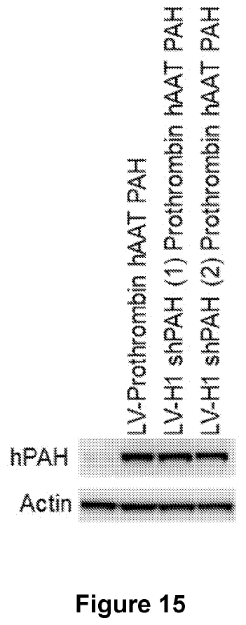

This Example illustrates that expression of human PAH is substantially increased in primary human hepatocytes with lentiviral vectors containing the prothrombin enhancer in combination with the hAAT promoter as shown in FIG. 9.

Human PAH, the prothrombin and ApoE enhancer, and hAAT promoter were synthesized by Eurofins Genomics (Louisville, Ky.) and inserted into a lentiviral vector. Insertion of the sequences was verified by DNA sequencing. The lentiviral vectors containing a verified hPAH sequence were then used to transduce primary human hepatocytes (Triangle Research Labs, North Carolina). The lentiviral vectors incorporated the coding sequence of human PAH or the coding sequence and 3′ UTR. In addition, hPAH expression in these constructs was driven by the liver-specific prothrombin or ApoE enhancer and hAAT promoter. Cells were transduced with lentiviral particles and after 4 days protein was analyzed by immunoblot for PAH expression. The relative expression of human PAH was detected by immunoblot using an anti-PAH antibody (Abcam, Cambridge, Mass.) and an anti-beta actin antibody (SigmaMillipore, Billerica, Mass.) was used for a loading control.

As shown in FIG. 9, five groups are compared: a control with a lentiviral vector containing only the prothrombin enhancer/hAAT promoter (lane 1), a lentiviral vector expressing the coding region of hPAH by the prothrombin enhancer/hAAT promoter (lane 2), a lentiviral vector expressing hPAH by the prothrombin enhancer/hAAT promoter and 5×HNF1 binding sites upstream of the prothrombin enhancer (lane 3), a lentiviral vector expressing hPAH by the prothrombin enhancer/hAAT promoter and containing a rabbit beta globin intron (lane 4), and a lentiviral vector expressing hPAH with the 3′ UTR by the ApoE/hAAT promoter (lane 5). FIG. 9 demonstrates that expression of PAH is increased in primary human hepatocytes when the prothrombin enhancer is combined with the hAAT promoter as compared with the ApoE enhancer. Also, inclusion of a rabbit beta globin intron sequence upstream of the hPAH coding sequence enhances hPAH expression.

### Example 12. Enzymatic Activity of Lentivirus-Delivered hPAH in Hepa1-6 Cells

This Example illustrates that lentiviral-delivered human PAH is enzymatically active as indicated by a decrease in phenylalanine (Phe) levels in the cell media and cell lysate when hPAH is expressed in Hepa1-6 cells as shown in FIGS. 10A and 10C, and 10B, respectively. A precursor of the cofactor BH4, sepiapterin, was required for PAH activity in Hepa1-6 cells.

Human PAH, the prothrombin and ApoE enhancer, hAAT promoter, and rabbit beta globin intron were synthesized and inserted into a lentiviral vector. Insertion of the sequences was verified by DNA sequencing. The lentiviral vectors containing a verified hPAH sequence were then used to transduce Hepa1-6 mouse liver cancer cells (American Type Culture Collection, Manassas, Va.). The lentiviral vectors incorporated the coding sequence of human PAH. In addition, hPAH expression in these constructs was driven by the liver-specific prothrombin enhancer/hAAT promoter and included the rabbit beta globin intron sequence. Cells were transduced with lentiviral particles and after 4 days phenylalanine levels were measured from either cell media or cell lysate using a Phenylalanine Assay kit (SigmaMillipore, Billerica, Mass.).

As shown in FIGS. 10A and 10B, four groups are compared from either cell media in FIG. 10A or from cell lysate in FIG. 10B: a control with a lentiviral vector containing only the prothrombin enhancer/hAAT promoter without sepiapterin (bar 1) or with sepiapterin (bar 3), a lentiviral vector expressing the coding region of hPAH by the prothrombin enhancer/hAAT promoter and containing a rabbit beta globin intron sequence without sepiapterin (bar 2) or with sepiapterin (bar 4). As shown in FIG. 10C, ten groups are compared from cell media: a control with a lentiviral vector containing only the prothrombin enhancer/hAAT promoter without sepiapterin (bar 1) or with sepiapterin (bar 6), a lentiviral vector expressing the coding region of hPAH by the prothrombin enhancer/hAAT promoter without sepiapterin (bar 2) or with sepiapterin (bar 7), a lentiviral vector expressing the coding region of hPAH by the prothrombin enhancer/hAAT promoter and 5×HNF1 binding sites upstream of the prothrombin enhancer without sepiapterin (bar 3) or with sepiapterin (bar 8), a lentiviral vector expressing the coding region of hPAH by the prothrombin enhancer/hAAT promoter and containing a rabbit beta globin intron sequence without sepiapterin (bar 4) or with sepiapterin (bar 9), a lentiviral vector expressing the coding region of hPAH including the 3′ UTR by the ApoE enhancer/hAAT promoter without sepiapterin (bar 5) or with sepiapterin (bar 10). FIGS. 10A and 10B show that hPAH is enzymatically active in Hepa1-6 cells in the presence of sepiapterin. There was a 38% decrease in cell media phenylalanine levels in Hepa1-6 cells transduced with a lentiviral vector expressing hPAH as shown in FIG. 10A. In cell lysate, there was an 88% decrease in phenylalanine levels in Hepa1-6 cells transduced with a lentiviral vector expressing hPAH as shown in FIG. 10B. FIG. 10C shows that hPAH is enzymatically active in vectors containing different liver-specific promoter elements. There was a 41% decrease in Phe levels with the vector containing the prothrombin enhancer/hAAT promoter and hPAH, a 43% decrease in Phe levels with the vector containing 5×HNF1 binding sites upstream of the prothrombin enhancer/hAAT promoter and hPAH, a 48% decrease in Phe levels with the vector containing the prothrombin enhancer/hAAT promoter including the rabbit beta globin intron and hPAH, and a 36% decrease in Phe levels with the vector containing the ApoE enhancer/hAAT promoter and hPAH including the 3′ UTR.

### Example 13. Lentiviral Delivered PAH Decreases Blood Phenylalanine Levels in Blood of PAH Mutant Mice

This Example illustrates that lentiviral-delivered PAH decreases phenylalanine levels in the blood of Pah^{enu2} mutant mice. The Pah^{enu2} mutant mouse is described and characterized in Shedlovsky et al. (Mouse Models of Human Phenylketonuria, Genetics 134: 1205-1210 (August, 1993)), the entirety of which is incorporated by reference herein. Pah^{enu2} mutant mice (n=4) were injected with 150 μl volume of the LV-Pro-hAAT-PAH (AGT323) vector via the tail vein. Control treated animals were injected via the tail vein with saline that did not contain a lentivirus vector. The titer of LV-Pro-hAAT-PAH (AGT323) was 1×10¹⁰ as determined by qPCR detection of integrated vector copies in transduced 293T cells. At the time of injection, the mice were 6-8 weeks old. Blood was collected before vector injection (T=0) and at 1 and 2 weeks post-injection.

For measurement of phenylalanine levels, blood was collected via the facial vein. A lancet was used to puncture the cheek and 400 μl of whole blood was collected in serum tubes. The blood was rested for one hour and then the tubes were centrifuged. The plasma/serum was separated on the top layer and collected. The plasma was then frozen until it was analyzed on a clinical amino acid analyzer instrument.

Introduction of the LV-Pro-hAAT-PAH (AGT323) vector caused a reduction in blood phenylalanine levels at both one week and two weeks post-injection. As shown in Table 1 and FIG. 11, the mean micro-molar levels of blood phenylalanine at the 1-week and 2-week time points was 1674.7 and 1890, respectively. This is compared to the mean micro-molar blood phenylalanine levels of control treated mice of between ˜2400 and ˜2500 (Table 1 and FIG. 11).

In addition, introduction of the LV-Pro-hAAT-PAH (AGT 323) vector resulted in genetic modification of the liver cells. Vector copy number studies on livers collected during necropsy showed the lentivirus vector copy number was approximately 0.2 per cell in these studies. As shown in FIG. 12, there is roughly a linear relationship between increasing vector copy number in liver and decreasing levels of phenylalanine (Pearson product correlation coefficient=−0.326).

A decrease in blood Phenylalanine levels of ≥20% would provide therapeutic benefit to human patients with Phenylketonuria disease, potentially shifting their diagnosis from classic PKU to mild Phenylalanemia.

TABLE 1
Blood Phenylalanine Suppression by Tail Vein Injection of
AGT323 in Juvenile enu2 mice

| Control Mice | Control | Control | Treated Mice | Treated/ | Treated/ |
|---|---|---|---|---|---|
| (n = 6) | Week 1 | Week 2 | (n = 12) | Week 1 | Week 2 |
| Mouse 1 | 2943* | 2165 | Mouse 7 | 1863 | 1448 |
| Mouse 2 | 2305 | 2155 | Mouse 8 | 1489 | 1412 |
| Mouse 3 | 2275 | 2480 | Mouse 9 | 1473 | 1741 |
| Mouse 4 | 2756 | 2432 | Mouse 10 | 1910 | 1999 |
| Mouse 5 | 2263 | 2701 | Mouse 11 | 1606 | 1753 |
| Mouse 6 | 2483 | 2477 | Mouse 12 | 1851 | 2337 |
| | | | Mouse 13 | 1187 | 1065 |
| | | | Mouse 14 | 2042 | 2266 |
| | | | Mouse 15 | 1841 | 2174 |
| | | | Mouse 16 | 1532 | 1498 |
| | | | Mouse 17 | 1768 | 1758 |
| | | | Mouse 18 | 1534 | 1534 |
| Mean | 2504.2 | 2401.7 | | 1674.7 | 1890 |
| Std Dev. of Mean | 230.2 | 161.1 | | 204.5 | 299.1 |

*Blood [Phe] in micromole/L

### Example 14. AAV-Delivered Expression of hPAH with Either a DJ or AAV2 Serotype in HEK293T Cells

This Example illustrates that human PAH is expressed in HEK293T cells with an AAV vector containing the prothrombin enhancer in combination with the hAAT promoter, and with or without a rabbit beta globin intron.

Human PAH, prothrombin enhancer, hAAT promoter, and rabbit beta globin intron were synthesized by Eurofins Genomics (Louisville, Ky.) and inserted into an AAV vector. Insertion of the sequences was verified by DNA sequencing. The AAV vectors containing a verified hPAH sequence were then used to transduce HEK293T cells (American Type Culture Collection, Manassas, Va.). The AAV vectors incorporated a human PAH gene as disclosed herein. In addition, hPAH expression in these constructs was driven by the liver-specific hAAT promoter and a rabbit beta globin intron. Cells were transduced with AAV particles and after three days, protein or RNA was analyzed by immunoblot or qPCR for PAH expression. The expression of human PAH protein was detected by immunoblot using an anti-PAH antibody (Abcam, Cambridge, Mass.) and an anti-beta actin antibody (MilliporeSigma, Billerica, Mass.) was used for a loading control. PAH RNA expression was detected by qPCR using a TaqMan Fam-labeled probe (SEQ ID NO: 66) and PAH primers (Fwd: SEQ ID NO: 67; Rev: SEQ ID NO: 68).

FIG. 13A shows a comparison of six groups: an AAV/DJ-GFP vector (lane 1), an AAV/DJ vector expressing hPAH by the prothrombin enhancer/hAAT promoter (lane 2), an AAV/DJ vector expressing hPAH by the prothrombin enhancer/hAAT promoter and a rabbit beta globin intron sequence (lane 3), an AAV2-GFP vector (lane 4), an AAV2 vector expressing hPAH by the prothrombin enhancer/hAAT promoter (lane 5), an AAV2 vector expressing hPAH by the prothrombin enhancer/hAAT promoter and a rabbit beta globin intron sequence (lane 6).

FIGS. 13B and 13C show blots after increased exposure the PAH bands in FIG. 13A. Exposure was increased so that the band densities were brought into a range to be analyzed by quantitative imaging. The original band intensities were much lower on the blot showing treatment with the AAV/2 serotype vector relative to the blot showing treatment with AAV/DJ serotype vector. As a result, a longer exposure time was needed on the AAV/2 serotype blot relative to the AAV/DJ serotype blot, in order to make quantitative measurements of PAH protein.

Results of quantitative imaging showed that PAH protein expression was increased by 2-fold using either an AAV/DJ vector that includes a rabbit beta globin intron or an AAV/DJ vector that lacks a rabbit beta globin intron (FIG. 13B; the numbers below the bands show relative fold increase). Using the AAV/2 vector with a rabbit beta globin intron suppressed expression of PAH, whereas using the AAV2 vector without a rabbit beta globin intron resulted in a 50-fold increase of PAH (FIG. 13C; the numbers below the bands show relative fold increase). Comparing PAH expression between AAV serotypes revealed that use of the AAV/DJ PAH vector results in higher expression of PAH protein relative to using the AAV2 PAH vector.

FIG. 13D demonstrates that PAH RNA expression is higher with the AAV/DJ PAH vector as compared with the AAV2 PAH vector, and there is similar expression with and without the rabbit beta globin intron.

### Example 15. Lentivirus Vector Therapy for PKU in Neonatal Enu2/Enu2 Mice

Neonatal enu2/enu2 mice (day 3 after birth) were treated with 10 μl of vector stock LV-Pro-hAAT-PAH via direct injection into the liver. Untreated animals received saline without vector (sham control). The vector stock was approximately 5×10⁸ transducing units per mL in sterile saline (measured in HEK293 cells) for a final does of ˜5×10⁶ transducing units per mouse (˜10⁹ transducing units per kg).

As shown in Table 2 and FIG. 14, micro-molar concentrations of blood phenylalanine levels was significantly reduced in LV-Pro-hAAT-PAH treated enu2/enu2 mice (1390+/−127) relative to untreated enu2/enu2 mice (2063+/−185).

TABLE 2

| | | enu2/enu2 | enu2/enu2 |
|---|---|---|---|
| | Wild-type | treated | untreated |
| | (n = 7) | (n = 5) | (n = 10) |
| Blood Phe (+/− | 60.7 +/− 3.4 | 1390 +/− 127 | 2063 +/− 185 |
| standard error of | | | |
| them mean) | | | |

| | |
|---|---|
| Treated vs. untreated | Unpaired t test P = |
| | 0.032 |
| | Difference between |
| | means blood [Phe]) |
| | 674 +/− 280 |

### Example 16. Expression of shPAH Targeting the 3′UTR of the PAH Acne does not Suppress PAH Expression by LV-H1-shPAH-Prothrombin-hAAT-hPAH in Hep3B Cells

This Example illustrates that shPAH does not suppress PAH expression from the lentiviral vector, LV-Pro-hAAT-PAH-shPAH sequence #1 (SEQ ID NO: 13), or from the lentiviral vector, LV-Pro-hAAT-PAH-shPAH sequence #2 (SEQ ID NO: 14), in Hep3B cells.

Human PAH as disclosed herein was synthesized and inserted into lentiviral vectors containing either PAH shRNA sequence #1 (SEQ ID NO: 13) or PAH shRNA sequence #2 (SEQ ID NO: 14). Insertion of the sequences was verified by DNA sequencing. Lentiviral vectors containing either PAH alone or in combination with either PAH shRNA sequence #1 (SEQ ID NO: 13) or PAH shRNA sequence #2 (SEQ ID NO: 14) were then used to transduce human Hep3B cells (purchased from American Type Culture Collection, Manassas, Va.). Cells were transduced with lentiviral particles and after 3 days protein was analyzed by western blot for PAH expression. The relative expression of human PAH was detected by immunoblot using an anti-PAH antibody (Abcam) and the loading control beta-actin. hPAH expression was driven by a prothrombin enhancer and hAAT promoter. The lentiviral vectors incorporated, in various instances, a human PAH gene and PAH shRNA sequence #1 (SEQ ID NO: 13) or PAH shRNA sequence #2 (SEQ ID NO: 14). Insertion of a shRNA sequence in the lentiviral vector (LV) was verified by DNA sequencing using a primer complementary to the promoter used to regulate shPAH-2 expression. In this case, an H1 promoter was used to regulate PAH shRNA expression. The target sequences for shPAH—(PAH shRNA sequence #1 (SEQ ID NO: 13) or PAH shRNA sequence #2 (SEQ ID NO: 14))—are in the PAH 3′UTR that are not present in the LV-PAH vector.

As shown in FIG. 15, expression of PAH derived from a lentivirus is not suppressed with either PAH shRNA sequence #1 (SEQ ID NO: 13) or PAH shRNA sequence #2 (SEQ ID NO: 14).

Sequence Listings

| SEQ | | |
|---|---|---|
| ID | | |
| NO: | Description | Sequence |
| 1 | PAH | ATGTCCACTGCGGTCCTGGAAAACCCAGGCTTGGGCAGGAAACTCT |
| | | CTGACTTTGGACAGGAAACAAGCTATATTGAAGACAACTGCAATCA |
| | | AAATGGTGCCATATCACTGATCTTCTCACTCAAAGAAGAAGTTGGTG |
| | | CATTGGCCAAAGTATTGCGCTTATTTGAGGAGAATGATGTAAACCTG |
| | | ACCCACATTGAATCTAGACCTTCTCGTTTAAAGAAAGATGAGTATGA |
| | | ATTTTTCACCCATTTGGATAAACGTAGCCTGCCTGCTCTGACAAACA |
| | | TCATCAAGATCTTGAGGCATGACATTGGTGCCACTGTCCATGAGCTT |
| | | TCACGAGATAAGAAGAAAGACACAGTGCCCTGGTTCCCAAGAACCA |
| | | TTCAAGAGCTGGACAGATTTGCCAATCAGATTCTCAGCTATGGAGCG |
| | | GAACTGGATGCTGACCACCCTGGTTTTAAAGATCCTGTGTACCGTGC |
| | | AAGACGGAAGCAGTTTGCTGACATTGCCTACAACTACCGCCATGGG |
| | | CAGCCCATCCCTCGAGTGGAATACATGGAGGAAGAAAAGAAAACAT |
| | | GGGGCACAGTGTTCAAGACTCTGAAGTCCTTGTATAAAACCCATGCT |
| | | TGCTATGAGTACAATCACATTTTTCCACTTCTTGAAAAGTACTGTGG |
| | | CTTCCATGAAGATAACATTCCCCAGCTGGAAGACGTTTCTCAATTCC |
| | | TGCAGACTTGCACTGGTTTCCGCCTCCGACCTGTGGCTGGCCTGCTTT |
| | | CCTCTCGGGATTTCTTGGGTGGCCTGGCCTTCCGAGTCTTCCACTGCA |
| | | CACAGTACATCAGACATGGATCCAAGCCCATGTATACCCCCGAACCT |
| | | GACATCTGCCATGAGCTGTTGGGACATGTGCCCTTGTTTTCAGATCG |
| | | CAGCTTTGCCCAGTTTTCCCAGGAAATTGGCCTTGCCTCTCTGGGTG |
| | | CACCTGATGAATACATTGAAAAGCTCGCCACAATTTACTGGTTTACT |
| | | GTGGAGTTTGGGCTCTGCAAACAAGGAGACTCCATAAAGGCATATG |
| | | GTGCTGGGCTCCTGTCATCCTTTGGTGAATTACAGTACTGCTTATCA |
| | | GAGAAGCCAAAGCTTCTCCCCCTGGAGCTGGAGAAGACAGCCATCC |
| | | AAAATTACACTGTCACGGAGTTCCAGCCCCTGTATTACGTGGCAGAG |
| | | AGTTTTAATGATGCCAAGGAGAAAGTAAGGAACTTTGCTGCCACAA |
| | | TACCTCGGCCCTTCTCAGTTCGCTACGACCCATACACCCAAAGGATT |
| | | GAGGTCTTGGACAATACCCAGCAGCTTAAGATTTTGGCTGATTCCAT |
| | | TAACAGTGAAATTGGAATCCTTTGCAGTGCCCTCCAGAAAATAAAGT |
| | | AA |
| 2 | Codon | ATGAGTACGGCTGTGCTCGAGAATCCAGGTTTGGGCCGAAAGCTGT |
| | optimized | CTGATTTTGGACAGGAGACATCTTATATTGAAGACAACTGCAACCAG |
| | PAH (Opt2) | AATGGTGCGATATCCCTTATTTTTTCTCTGAAAGAAGAAGTAGGTGC |
| | | GCTGGCAAAGGTCTTGCGGCTGTTTGAAGAGAACGATGTTAATCTTA |
| | | CTCATATTGAGTCCAGACCATCACGGCTGAAAAAAGACGAGTACGA |
| | | ATTTTTTACTCACTTGGACAAACGAAGCTTGCCGGCTCTTACTAATA |
| | | TCATTAAGATCCTCCGGCATGACATAGGGGCGACAGTGCATGAGCTT |
| | | TCAAGGGATAAAAAGAAAGATACCGTCCCCTGGTTTCCAAGGACCA |
| | | TACAAGAACTCGACCGATTCGCGAACCAGATCCTTTCATATGGTGCT |
| | | GAGTTGGATGCTGACCACCCCGGCTTCAAAGACCCGGTCTACCGAG |
| | | CGCGGCGGAAACAATTTGCTGACATCGCATACAATTACAGGCATGG |
| | | CCAGCCAATTCCTAGAGTAGAATACATGGAAGAAGAGAAAAAAACC |
| | | TGGGGTACCGTCTTCAAGACGCTGAAATCATTGTATAAAACTCATGC |
| | | ATGTTACGAATATAACCATATTTTTCCGTTGCTCGAGAAATATTGCG |
| | | GGTTCCACGAAGATAACATCCCACAACTCGAGGATGTATCTCAGTTC |
| | | CTCCAGACCTGTACGGGGTTTCGACTTAGGCCTGTCGCGGGTTTGCT |
| | | CAGTTCTCGAGACTTCCTGGGTGGATTGGCGTTTCGGGTATTCCATT |
| | | GCACGCAGTATATCCGACACGGAAGTAAGCCAATGTACACGCCAGA |
| | | ACCCGATATCTGTCACGAATTGCTTGGACACGTTCCTCTGTTTTCTGA |
| | | TCGATCATTCGCTCAGTTTTCACAGGAAATCGGCCTGGCATCTTTGG |
| | | GAGCGCCGGATGAATATATTGAGAAGCTCGCTACAATTTACTGGTTC |
| | | ACGGTAGAATTTGGGTTGTGCAAGCAGGGTGATAGTATTAAAGCAT |
| | | ACGGTGCGGGATTGCTGTCCTCATTCGGGGAGCTTCAGTATTGCCTG |
| | | TCCGAGAAACCCAAGCTGTTGCCGTTGGAATTGGAAAAAACCGCTA |
| | | TCCAAAATTACACAGTAACGGAGTTCCAACCTTTGTACTACGTAGCC |
| | | GAGTCATTTAACGATGCAAAGGAGAAGGTCAGAAATTTTGCTGCGA |
| | | CGATACCCAGACCGTTCTCAGTAAGGTACGATCCTTACACTCAGAGG |
| | | ATTGAAGTCCTGGATAATACGCAACAGCTCAAGATCCTGGCAGACT |
| | | CCATAAATTCTGAAATCGGCATCTTGTGTTCAGCACTGCAAAAGATA |
| | | AAATAA |
| 3 | PAH 3′UTR | AGCCATGGACAGAATGTGGTCTGTCAGCTGTGAATCTGTTGATGGAG |
| | sequence (897 | ATCCAACTATTTCTTTCATCAGAAAAAGTCCGAAAAGCAAACCTTAA |
| | nucleotides) | TTTGAAATAACAGCCTTAAATCCTTTACAAGATGGAGAAACAACAA |
| | | ATAAGTCAAAATAATCTGAAATGACAGGATATGAGTACATACTCAA |
| | | GAGCATAATGGTAAATCTTTTGGGGTCATCTTTGATTTAGAGATGAT |
| | | AATCCCATACTCTCAATTGAGTTAAATCAGTAATCTGTCGCATTTCA |
| | | TCAAGATTAATTAAAATTTGGGACCTGCTTCATTCAAGCTTCATATA |
| | | TGCTTTGCAGAGAACTCATAAAGGAGCATATAAGGCTAAATGTAAA |
| | | ACCCAAGACTGTCATTAGAATTGAATTATTGGGCTTAATATAAATCG |
| | | TAACCTATGAAGTTTATTTTTTATTTTAGTTAACTATGATTCCAATTA |
| | | CTACTTTGTTATTGTACCTAAGTAAATTTTCTTTAAGTCAGAAGCCCA |
| | | TTAAAATAGTTACAAGCATTGAACTTCTTTAGTATTATATTAATATA |
| | | AAAACATTTTTGTATGTTTTATTGTAATCATAAATACTGCTGTATAAG |
| | | GTAATAAAACTCTGCACCTAATCCCCATAACTTCCAGTATCATTTTC |
| | | CAATTAATTATCAAGTCTGTTTTGGGAAACACTTTGAGGACATTTAT |
| | | GATGCAGCAGATGTTGACTAAAGGCTTGGTTGGTAGATATTCAGGA |
| | | AATGTTCACTGAATAAATAAGTAAATACATTATTGAAAAGCAAATCT |
| | | GTATAAATGTGAAATTTTTATTTGTATTAGTAATAAAACATTAGTAG |
| | | TTTAAACAAAAAAAAAAAAAAAAAAAAAAAAAAAAAAACTCGACT |
| | | CTAGATT |
| 4 | PAH 3′UTR | AGCCATGGACAGAATGTGGTCTGTCAGCTGTGAATCTGTTGATGGAG |
| | sequence (289 | ATCCAACTATTTCTTTCATCAGAAAAAGTCCGAAAAGCAAACCTTAA |
| | nucleotides) | TTTGAAATAACAGCCTTAAATCCTTTACAAGATGGAGAAACAACAA |
| | | ATAAGTCAAAATAATCTGAAATGACAGGATATGAGTACATACTCAA |
| | | GAGCATAATGGTAAATCTTTTGGGGTCATCTTTGATTTAGAGATGAT |
| | | AATCCCATACTCTCAATTGAGTTAAATCAGTAATCTGTCGCATTTCA |
| | | TCAAGATTA |
| 5 | Prothrombin | GCGAGAACTTGTGCCTCCCCGTGTTCCTGCTCTTTGTCCCTCTGTCCT |
| | enhancer (Pro) | ACTTAGACTAATATTTGCCTTGGGTACTGCAAACAGGAAATGGGGG |
| | | AGGGACAGGAGTAGGGCGGAGGGTAG |
| 6 | Human alpha-1 | GATCTTGCTACCAGTGGAACAGCCACTAAGGATTCTGCAGTGAGAG |
| | antitrypsin | CAGAGGGCCAGCTAAGTGGTACTCTCCCAGAGACTGTCTGACTCAC |
| | promoter | GCCACCCCCTCCACCTTGGACACAGGACGCTGTGGTTTCTGAGCCAG |
| | (hAAT) | GTACAATGACTCCTTTCGGTAAGTGCAGTGGAAGCTGTACACTGCCC |
| | | AGGCAAAGCGTCCGGGCAGCGTAGGCGGGCGACTCAGATCCCAGCC |
| | | AGTGGACTTAGCCCCTGTTTGCTCCTCCGATAACTGGGGTGACCTTG |
| | | GTTAATATTCACCAGCAGCCTCCCCCGTTGCCCCTCTGGATCCACTG |
| | | CTTAAATACGGACGAGGACAGGGCCCTGTCTCCTCAGCTTCAGGCAC |
| | | CACCACTGACCTGGGACAGTGAAT |
| 7 | Rabbit beta | GTGAGTTTGGGGACCCTTGATTGTTCTTTCTTTTTCGCTATTGTAAAA |
| | globin intron | TTCATGTTATATGGAGGGGGCAAAGTTTTCAGGGTGTTGTTTAGAAT |
| | | GGGAAGATGTCCCTTGTATCACCATGGACCCTCATGATAATTTTGTT |
| | | TCTTTCACTTTCTACTCTGTTGACAACCATTGTCTCCTCTTATTTTCTT |
| | | TTCATTTTCTGTAACTTTTTCGTTAAACTTTAGCTTGCATTTGTAACG |
| | | AATTTTTAAATTCACTTTTGTTTATTTGTCAGATTGTAAGTACTTTCT |
| | | CTAATCACTTTTTTTTCAAGGCAATCAGGGTATATTATATTGTACTTC |
| | | AGCACAGTTTTAGAGAACAATTGTTATAATTAAATGATAAGGTAGA |
| | | ATATTTCTGCATATAAATTCTGGCTGGCGTGGAAATATTCTTATTGGT |
| | | AGAAACAACTACACCCTGGTCATCATCCTGCCTTTCTCTTTATGGTTA |
| | | CAATGATATACACTGTTTGAGATGAGGATAAAATACTCTGAGTCCAA |
| | | ACCGGGCCCCTCTGCTAACCATGTTCATGCCTTCTTCTCTTTCCTACA |
| | | G |
| 8 | Human beta | GGATCCTGAGAACTTCAGGGTGAGTCTATGGGACGCTTGATGTTTTC |
| | globin intron | TTTCCCCTTCTTTTCTATGGTTAAGTTCATGTCATAGGAAGGGGATAA |
| | | GTAACAGGGTACACATATTGACCAAATCAGGGTAATTTTGCATTTGT |
| | | AATTTTAAAAAATGCTTTCTTCTTTTAATATACTTTTTTGTTTATCTTA |
| | | TTTCTAATACTTTCCCTAATCTCTTTCTTTCAGGGCAATAATGATACA |
| | | ATGTATCATGCCTCTTTGCACCATTCTAAAGAATAACAGTGATAATT |
| | | TCTGGGTTAAGGCAATAGCAATATTTCTGCATATAAATATTTCTGCA |
| | | TATAAATTGTAACTGATGTAAGAGGTTTCATATTGCTAATAGCAGCT |
| | | ACAATCCAGCTACCATTCTGCTTTTATTTTATGGTTGGGATAAGGCT |
| | | GGATTATTCTGAGTCCAAGCTAGGCCCTTTTGCTAATCATGTTCATA |
| | | CCTCTTATCTTCCTCCCACAGCTCCTGGGCAACGTGCTGGTCTGTGTG |
| | | CTGGCCCATCACTTTGGCAAAG |
| 9 | 1X | GTTAATCATTAAC |
| | Hepatocyte | |
| | Nuclear Factor | |
| | 1 (1XHNF1) | |
| 10 | 5X | GTTAATCATTAACGTTAATCATTAACGTTAATCATTAACGTTAATCA |
| | Hepatocyte | TTAACGTTAATCATTAAC |
| | Nuclear Factor | |
| | 1 (5XHNF1) | |
| 11 | 1X | GTTAATCATTAACGCTTGTACTTTGGTACA |
| | Hepatocyte | |
| | Nuclear Factor | |
| | 1/4 | |
| | (1XHNF1/4) | |
| 12 | 3X | GTTAATCATTAACGCTTGTACTTTGGTACAGTTAATCATTAACGCTTG |
| | Hepatocyte | TACTTTGGTACAGTTAATCATTAACGCTTGTACTTTGGTACA |
| | Nuclear Factor | |
| | 1/4 | |
| | (3XHNF1/4) | |
| 13 | PAH shRNA | TCGCATTTCATCAAGATTAATCTCGAGATTAATCTTGATGAAATGCG |
| | sequence #1 | ATTTTT |
| 14 | PAH shRNA | ACTCATAAAGGAGCATATAAGCTCGAGCTTATATGCTCCTTTATGAG |
| | sequence #2 | TTTTTT |
| 15 | Rous Sarcoma | GTAGTCTTATGCAATACTCTTGTAGTCTTGCAACATGGTAACGATGA |
| | virus (RSV) | GTTAGCAACATGCCTTACAAGGAGAGAAAAAGCACCGTGCATGCCG |
| | promoter | ATTGGTGGAAGTAAGGTGGTACGATCGTGCCTTATTAGGAAGGCAA |
| | | CAGACGGGTCTGACATGGATTGGACGAACCACTGAATTGCCGCATT |
| | | GCAGAGATATTGTATTTAAGTGCCTAGCTCGATACAATAAACG |
| 16 | 5′ Long | GGTCTCTCTGGTTAGACCAGATCTGAGCCTGGGAGCTCTCTGGCTAA |
| | terminal | CTAGGGAACCCACTGCTTAAGCCTCAATAAAGCTTGCCTTGAGTGCT |
| | repeat (LTR) | TCAAGTAGTGTGTGCCCGTCTGTTGTGTGACTCTGGTAACTAGAGAT |
| | | CCCTCAGACCCTTTTAGTCAGTGTGGAAAATCTCTAGCA |
| 17 | Psi Packaging | TACGCCAAAAATTTTGACTAGCGGAGGCTAGAAGGAGAGAG |
| | signal | |
| 18 | Rev response | AGGAGCTTTGTTCCTTGGGTTCTTGGGAGCAGCAGGAAGCACTATGG |
| | element | GCGCAGCCTCAATGACGCTGACGGTACAGGCCAGACAATTATTGTCT |
| | (RRE) | GGTATAGTGCAGCAGCAGAACAATTTGCTGAGGGCTATTGAGGCGC |
| | | AACAGCATCTGTTGCAACTCACAGTCTGGGGCATCAAGCAGCTCCA |
| | | GGCAAGAATCCTGGCTGTGGAAAGATACCTAAAGGATCAACAGCTC |
| | | C |
| 19 | Central | TTTTAAAAGAAAAGGGGGGATTGGGGGGTACAGTGCAGGGGAAAG |
| | polypurine | AATAGTAGACATAATAGCAACAGACATACAAACTAAAGAATTACAA |
| | tract (cPPT) | AAACAAATTACAAAATTCAAAATTTTA |
| 20 | Long WPRE | AATCAACCTCTGATTACAAAATTTGTGAAAGATTGACTGGTATTCTT |
| | sequence | AACTATGTTGCTCCTTTTACGCTATGTGGATACGCTGCTTTAATGCCT |
| | | TTGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGTC |
| | | AGGCAACGTGGCGTGGTGTGCACTGTGTTTGCTGACGCAACCCCCAC |
| | | TGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTG |
| | | CCCGCTGCTGGACAGGGGCTCGGCTGTTGGGCACTGACAATTCCGTG |
| | | GTGTTGTCGGGGAAATCATCGTCCTTTCCTTGGCTGCTCGCCTGTGTT |
| | | GCCACCTGGATTCTGCGCGGGACGTCCTTCTGCTACGTCCCTTCGGC |
| | | CCTCAATCCAGCGGACCTTCCTTCCCGCGGCCTGCTGCCGGCTCTGC |
| | | GGCCTCTTCCGCGTCTTCGCCTTCGCCCTCAGACGAGTCGGATCTCC |
| | | CTTTGGGCCGCCTCCCCGCCT |
| 21 | delta U3 | TGGAAGGGCTAATTCACTCCCAACGAAGATAAGATCTGCTTTTTGCT |
| | 3′LTR | TGTACTGGGTCTCTCTGGTTAGACCAGATCTGAGCCTGGGAGCTCTC |
| | | TGGCTAACTAGGGAACCCACTGCTTAAGCCTCAATAAAGCTTGCCTT |
| | | GAGTGCTTCAAGTAGTGTGTGCCCGTCTGTTGTGTGACTCTGGTAAC |
| | | TAGAGATCCCTCAGACCCTTTTAGTCAGTGTGGAAAATCTCTAGCAG |
| | | TAGTAGTTCATGTCA |
| 22 | H1 Promoter | GAACGCTGACGTCATCAACCCGCTCCAAGGAATCGCGGGCCCAGTG |
| | | TCACTAGGCGGGAACACCCAGCGCGCGTGCGCCCTGGCAGGAAGAT |
| | | GGCTGTGAGGGACAGGGGAGTGGCGCCCTGCAATATTTGCATGTCG |
| | | CTATGTGTTCTGGGAAATCACCATAAACGTGAAATGTCTTTGGATTT |
| | | GGGAATCTTATAAGTTCTGTATGAGACCACTT |
| 23 | CMV | TAGTTATTAATAGTAATCAATTACGGGGTCATTAGTTCATAGCCCAT |
| | enhancer/ | ATATGGAGTTCCGCGTTACATAACTTACGGTAAATGGCCCGCCTGGC |
| | chicken | TGACCGCCCAACGACCCCCGCCCATTGACGTCAATAATGACGTATGT |
| | beta actin | TCCCATAGTAACGCCAATAGGGACTTTCCATTGACGTCAATGGGTGG |
| | promoter | ACTATTTACGGTAAACTGCCCACTTGGCAGTACATCAAGTGTATCAT |
| | | ATGCCAAGTACGCCCCCTATTGACGTCAATGACGGTAAATGGCCCGC |
| | | CTGGCATTATGCCCAGTACATGACCTTATGGGACTTTCCTACTTGGC |
| | | AGTACATCTACGTATTAGTCATCGCTATTACCATGGGTCGAGGTGAG |
| | | CCCCACGTTCTGCTTCACTCTCCCCATCTCCCCCCCCTCCCCACCCCC |
| | | AATTTTGTATTTATTTATTTTTTAATTATTTTGTGCAGCGATGGGGGC |
| | | GGGGGGGGGGGGGGCGCGCGCCAGGCGGGGCGGGGCGGGGCGAGG |
| | | GGCGGGGCGGGGCGAGGCGGAGAGGTGCGGCGGCAGCCAATCAGA |
| | | GCGGCGCGCTCCGAAAGTTTCCTTTTATGGCGAGGCGGCGGCGGCG |
| | | GCGGCCCTATAAAAAGCGAAGCGCGCGGCGGGCG |
| 24 | HIV Gag | ATGGGTGCGAGAGCGTCAGTATTAAGCGGGGGAGAATTAGATCGAT |
| | | GGGAAAAAATTCGGTTAAGGCCAGGGGGAAAGAAAAAATATAAAT |
| | | TAAAACATATAGTATGGGCAAGCAGGGAGCTAGAACGATTCGCAGT |
| | | TAATCCTGGCCTGTTAGAAACATCAGAAGGCTGTAGACAAATACTG |
| | | GGACAGCTACAACCATCCCTTCAGACAGGATCAGAAGAACTTAGAT |
| | | CATTATATAATACAGTAGCAACCCTCTATTGTGTGCATCAAAGGATA |
| | | GAGATAAAAGACACCAAGGAAGCTTTAGACAAGATAGAGGAAGAG |
| | | CAAAACAAAAGTAAGAAAAAAGCACAGCAAGCAGCAGCTGACACA |
| | | GGACACAGCAATCAGGTCAGCCAAAATTACCCTATAGTGCAGAACA |
| | | TCCAGGGGCAAATGGTACATCAGGCCATATCACCTAGAACTTTAAAT |
| | | GCATGGGTAAAAGTAGTAGAAGAGAAGGCTTTCAGCCCAGAAGTGA |
| | | TACCCATGTTTTCAGCATTATCAGAAGGAGCCACCCCACAAGATTTA |
| | | AACACCATGCTAAACACAGTGGGGGGACATCAAGCAGCCATGCAAA |
| | | TGTTAAAAGAGACCATCAATGAGGAAGCTGCAGAATGGGATAGAGT |
| | | GCATCCAGTGCATGCAGGGCCTATTGCACCAGGCCAGATGAGAGAA |
| | | CCAAGGGGAAGTGACATAGCAGGAACTACTAGTACCCTTCAGGAAC |
| | | AAATAGGATGGATGACACATAATCCACCTATCCCAGTAGGAGAAAT |
| | | CTATAAAAGATGGATAATCCTGGGATTAAATAAAATAGTAAGAATG |
| | | TATAGCCCTACCAGCATTCTGGACATAAGACAAGGACCAAAGGAAC |
| | | CCTTTAGAGACTATGTAGACCGATTCTATAAAACTCTAAGAGCCGAG |
| | | CAAGCTTCACAAGAGGTAAAAAATTGGATGACAGAAACCTTGTTGG |
| | | TCCAAAATGCGAACCCAGATTGTAAGACTATTTTAAAAGCATTGGG |
| | | ACCAGGAGCGACACTAGAAGAAATGATGACAGCATGTCAGGGAGTG |
| | | GGGGGACCCGGCCATAAAGCAAGAGTTTTGGCTGAAGCAATGAGCC |
| | | AAGTAACAAATCCAGCTACCATAATGATACAGAAAGGCAATTTTAG |
| | | GAACCAAAGAAAGACTGTTAAGTGTTTCAATTGTGGCAAAGAAGGG |
| | | CACATAGCCAAAAATTGCAGGGCCCCTAGGAAAAAGGGCTGTTGGA |
| | | AATGTGGAAAGGAAGGACACCAAATGAAAGATTGTACTGAGAGAC |
| | | AGGCTAATTTTTTAGGGAAGATCTGGCCTTCCCACAAGGGAAGGCC |
| | | AGGGAATTTTCTTCAGAGCAGACCAGAGCCAACAGCCCCACCAGAA |
| | | GAGAGCTTCAGGTTTGGGGAAGAGACAACAACTCCCTCTCAGAAGC |
| | | AGGAGCCGATAGACAAGGAACTGTATCCTTTAGCTTCCCTCAGATCA |
| | | CTCTTTGGCAGCGACCCCTCGTCACAATAA |
| 25 | HIV Pol | ATGAATTTGCCAGGAAGATGGAAACCAAAAATGATAGGGGGAATTG |
| | | GAGGTTTTATCAAAGTAGGACAGTATGATCAGATACTCATAGAAAT |
| | | CTGCGGACATAAAGCTATAGGTACAGTATTAGTAGGACCTACACCT |
| | | GTCAACATAATTGGAAGAAATCTGTTGACTCAGATTGGCTGCACTTT |
| | | AAATTTTCCCATTAGTCCTATTGAGACTGTACCAGTAAAATTAAAGC |
| | | CAGGAATGGATGGCCCAAAAGTTAAACAATGGCCATTGACAGAAGA |
| | | AAAAATAAAAGCATTAGTAGAAATTTGTACAGAAATGGAAAAGGAA |
| | | GGAAAAATTTCAAAAATTGGGCCTGAAAATCCATACAATACTCCAG |
| | | TATTTGCCATAAAGAAAAAAGACAGTACTAAATGGAGAAAATTAGT |
| | | AGATTTCAGAGAACTTAATAAGAGAACTCAAGATTTCTGGGAAGTT |
| | | CAATTAGGAATACCACATCCTGCAGGGTTAAAACAGAAAAAATCAG |
| | | TAACAGTACTGGATGTGGGCGATGCATATTTTTCAGTTCCCTTAGAT |
| | | AAAGACTTCAGGAAGTATACTGCATTTACCATACCTAGTATAAACAA |
| | | TGAGACACCAGGGATTAGATATCAGTACAATGTGCTTCCACAGGGA |
| | | TGGAAAGGATCACCAGCAATATTCCAGTGTAGCATGACAAAAATCT |
| | | TAGAGCCTTTTAGAAAACAAAATCCAGACATAGTCATCTATCAATAC |
| | | ATGGATGATTTGTATGTAGGATCTGACTTAGAAATAGGGCAGCATA |
| | | GAACAAAAATAGAGGAACTGAGACAACATCTGTTGAGGTGGGGATT |
| | | TACCACACCAGACAAAAAACATCAGAAAGAACCTCCATTCCTTTGG |
| | | ATGGGTTATGAACTCCATCCTGATAAATGGACAGTACAGCCTATAGT |
| | | GCTGCCAGAAAAGGACAGCTGGACTGTCAATGACATACAGAAATTA |
| | | GTGGGAAAATTGAATTGGGCAAGTCAGATTTATGCAGGGATTAAAG |
| | | TAAGGCAATTATGTAAACTTCTTAGGGGAACCAAAGCACTAACAGA |
| | | AGTAGTACCACTAACAGAAGAAGCAGAGCTAGAACTGGCAGAAAA |
| | | CAGGGAGATTCTAAAAGAACCGGTACATGGAGTGTATTATGACCCA |
| | | TCAAAAGACTTAATAGCAGAAATACAGAAGCAGGGGCAAGGCCAAT |
| | | GGACATATCAAATTTATCAAGAGCCATTTAAAAATCTGAAAACAGG |
| | | AAAATATGCAAGAATGAAGGGTGCCCACACTAATGATGTGAAACAA |
| | | TTAACAGAGGCAGTACAAAAAATAGCCACAGAAAGCATAGTAATAT |
| | | GGGGAAAGACTCCTAAATTTAAATTACCCATACAAAAGGAAACATG |
| | | GGAAGCATGGTGGACAGAGTATTGGCAAGCCACCTGGATTCCTGAG |
| | | TGGGAGTTTGTCAATACCCCTCCCTTAGTGAAGTTATGGTACCAGTT |
| | | AGAGAAAGAACCCATAATAGGAGCAGAAACTTTCTATGTAGATGGG |
| | | GCAGCCAATAGGGAAACTAAATTAGGAAAAGCAGGATATGTAACTG |
| | | ACAGAGGAAGACAAAAAGTTGTCCCCCTAACGGACACAACAAATCA |
| | | GAAGACTGAGTTACAAGCAATTCATCTAGCTTTGCAGGATTCGGGAT |
| | | TAGAAGTAAACATAGTGACAGACTCACAATATGCATTGGGAATCAT |
| | | TCAAGCACAACCAGATAAGAGTGAATCAGAGTTAGTCAGTCAAATA |
| | | ATAGAGCAGTTAATAAAAAAGGAAAAAGTCTACCTGGCATGGGTAC |
| | | CAGCACACAAAGGAATTGGAGGAAATGAACAAGTAGATGGGTTGGT |
| | | CAGTGCTGGAATCAGGAAAGTACTA |
| 26 | HIV Int | TTTTTAGATGGAATAGATAAGGCCCAAGAAGAACATGAGAAATATC |
| | | ACAGTAATTGGAGAGCAATGGCTAGTGATTTTAACCTACCACCTGTA |
| | | GTAGCAAAAGAAATAGTAGCCAGCTGTGATAAATGTCAGCTAAAAG |
| | | GGGAAGCCATGCATGGACAAGTAGACTGTAGCCCAGGAATATGGCA |
| | | GCTAGATTGTACACATTTAGAAGGAAAAGTTATCTTGGTAGCAGTTC |
| | | ATGTAGCCAGTGGATATATAGAAGCAGAAGTAATTCCAGCAGAGAC |
| | | AGGGCAAGAAACAGCATACTTCCTCTTAAAATTAGCAGGAAGATGG |
| | | CCAGTAAAAACAGTACATACAGACAATGGCAGCAATTTCACCAGTA |
| | | CTACAGTTAAGGCCGCCTGTTGGTGGGCGGGGATCAAGCAGGAATT |
| | | TGGCATTCCCTACAATCCCCAAAGTCAAGGAGTAATAGAATCTATGA |
| | | ATAAAGAATTAAAGAAAATTATAGGACAGGTAAGAGATCAGGCTGA |
| | | ACATCTTAAGACAGCAGTACAAATGGCAGTATTCATCCACAATTTTA |
| | | AAAGAAAAGGGGGGATTGGGGGGTACAGTGCAGGGGAAAGAATAG |
| | | TAGACATAATAGCAACAGACATACAAACTAAAGAATTACAAAAACA |
| | | AATTACAAAAATTCAAAATTTTCGGGTTTATTACAGGGACAGCAGA |
| | | GATCCAGTTTGGAAAGGACCAGCAAAGCTCCTCTGGAAAGGTGAAG |
| | | GGGCAGTAGTAATACAAGATAATAGTGACATAAAAGTAGTGCCAAG |
| | | AAGAAAAGCAAAGATCATCAGGGATTATGGAAAACAGATGGCAGG |
| | | TGATGATTGTGTGGCAAGTAGACAGGATGAGGATTAA |
| 27 | HIV RRE | AGGAGCTTTGTTCCTTGGGTTCTTGGGAGCAGCAGGAAGCACTATGG |
| | | GCGCAGCGTCAATGACGCTGACGGTACAGGCCAGACAATTATTGTC |
| | | TGGTATAGTGCAGCAGCAGAACAATTTGCTGAGGGCTATTGAGGCG |
| | | CAACAGCATCTGTTGCAACTCACAGTCTGGGGCATCAAGCAGCTCCA |
| | | GGCAAGAATCCTGGCTGTGGAAAGATACCTAAAGGATCAACAGCTC |
| | | CT |
| 28 | HIV Rev | ATGGCAGGAAGAAGCGGAGACAGCGACGAAGAACTCCTCAAGGCA |
| | | GTCAGACTCATCAAGTTTCTCTATCAAAGCAACCCACCTCCCAATCC |
| | | CGAGGGGACCCGACAGGCCCGAAGGAATAGAAGAAGAAGGTGGAG |
| | | AGAGAGACAGAGACAGATCCATTCGATTAGTGAACGGATCCTTAGC |
| | | ACTTATCTGGGACGATCTGCGGAGCCTGTGCCTCTTCAGCTACCACC |
| | | GCTTGAGAGACTTACTCTTGATTGTAACGAGGATTGTGGAACTTCTG |
| | | GGACGCAGGGGGTGGGAAGCCCTCAAATATTGGTGGAATCTCCTAC |
| | | AATATTGGAGTCAGGAGCTAAAGAATAG |
| 29 | CMV | ACATTGATTATTGACTAGTTATTAATAGTAATCAATTACGGGGTCAT |
| | Promoter | TAGTTCATAGCCCATATATGGAGTTCCGCGTTACATAACTTACGGTA |
| | | AATGGCCCGCCTGGCTGACCGCCCAACGACCCCCGCCCATTGACGTC |
| | | AATAATGACGTATGTTCCCATAGTAACGCCAATAGGGACTTTCCATT |
| | | GACGTCAATGGGTGGAGTATTTACGGTAAACTGCCCACTTGGCAGTA |
| | | CATCAAGTGTATCATATGCCAAGTACGCCCCCTATTGACGTCAATGA |
| | | CGGTAAATGGCCCGCCTGGCATTATGCCCAGTACATGACCTTATGGG |
| | | ACTTTCCTACTTGGCAGTACATCTACGTATTAGTCATCGCTATTACCA |
| | | TGGTGATGCGGTTTTGGCAGTACATCAATGGGCGTGGATAGCGGTTT |
| | | GACTCACGGGGATTTCCAAGTCTCCACCCCATTGACGTCAATGGGAG |
| | | TTTGTTTTGGCACCAAAATCAACGGGACTTTCCAAAATGTCGTAACA |
| | | ACTCCGCCCCATTGACGCAAATGGGCGGTAGGCGTGTACGGTGGGA |
| | | GGTCTATATAAGCAGAGCTCTCTGGCTAACTAGAGAACCCACTGCTT |
| | | ACTG |
| 30 | VSV-G | ATGAAGTGCCTTTTGTACTTAGCCTTTTTATTCATTGGGGTGAATTGC |
| | Envelope | AAGTTCACCATAGTTTTTCCACACAACCAAAAAGGAAACTGGAAAA |
| | Glycoprotein | ATGTTCCTTCTAATTACCATTATTGCCCGTCAAGCTCAGATTTAAATT |
| | | GGCATAATGACTTAATAGGCACAGCCTTACAAGTCAAAATGCCCAA |
| | | GAGTCACAAGGCTATTCAAGCAGACGGTTGGATGTGTCATGCTTCCA |
| | | AATGGGTCACTACTTGTGATTTCCGCTGGTATGGACCGAAGTATATA |
| | | ACACATTCCATCCGATCCTTCACTCCATCTGTAGAACAATGCAAGGA |
| | | AAGCATTGAACAAACGAAACAAGGAACTTGGCTGAATCCAGGCTTC |
| | | CCTCCTCAAAGTTGTGGATATGCAACTGTGACGGATGCCGAAGCAGT |
| | | GATTGTCCAGGTGACTCCTCACCATGTGCTGGTTGATGAATACACAG |
| | | GAGAATGGGTTGATTCACAGTTCATCAACGGAAAATGCAGCAATTA |
| | | CATATGCCCCACTGTCCATAACTCTACAACCTGGCATTCTGACTATA |
| | | AGGTCAAAGGGCTATGTGATTCTAACCTCATTTCCATGGACATCACC |
| | | TTCTTCTCAGAGGACGGAGAGCTATCATCCCTGGGAAAGGAGGGCA |
| | | CAGGGTTCAGAAGTAACTACTTTGCTTATGAAACTGGAGGCAAGGC |
| | | CTGCAAAATGCAATACTGCAAGCATTGGGGAGTCAGACTCCCATCA |
| | | GGTGTCTGGTTCGAGATGGCTGATAAGGATCTCTTTGCTGCAGCCAG |
| | | ATTCCCTGAATGCCCAGAAGGGTCAAGTATCTCTGCTCCATCTCAGA |
| | | CCTCAGTGGATGTAAGTCTAATTCAGGACGTTGAGAGGATCTTGGAT |
| | | TATTCCCTCTGCCAAGAAACCTGGAGCAAAATCAGAGCGGGTCTTCC |
| | | AATCTCTCCAGTGGATCTCAGCTATCTTGCTCCTAAAAACCCAGGAA |
| | | CCGGTCCTGCTTTCACCATAATCAATGGTACCCTAAAATACTTTGAG |
| | | ACCAGATACATCAGAGTCGATATTGCTGCTCCAATCCTCTCAAGAAT |
| | | GGTCGGAATGATCAGTGGAACTACCACAGAAAGGGAACTGTGGGAT |
| | | GACTGGGCACCATATGAAGACGTGGAAATTGGACCCAATGGAGTTC |
| | | TGAGGACCAGTTCAGGATATAAGTTTCCTTTATACATGATTGGACAT |
| | | GGTATGTTGGACTCCGATCTTCATCTTAGCTCAAAGGCTCAGGTGTT |
| | | CGAACATCCTCACATTCAAGACGCTGCTTCGCAACTTCCTGATGATG |
| | | AGAGTTTATTTTTTGGTGATACTGGGCTATCCAAAAATCCAATCGAG |
| | | CTTGTAGAAGGTTGGTTCAGTAGTTGGAAAAGCTCTATTGCCTCTTT |
| | | TTTCTTTATCATAGGGTTAATCATTGGACTATTCTTGGTTCTCCGAGT |
| | | TGGTATCCATCTTTGCATTAAATTAAAGCACACCAAGAAAAGACAG |
| | | ATTTATACAGACATAGAGATGAACCGACTTGGAAAGTGA |
| 31 | Left ITR | CCTGCAGGCAGCTGCGCGCTCGCTCGCTCACTGAGGCCGCCCGGGC |
| | | AAAGCCCGGGCGTCGGGCGACCTTTGGTCGCCCGGCCTCAGTGAGC |
| | | GAGCGAGCGCGCAGAGAGGGAGTGGCCAACTCCATCACTAGGGGTT |
| | | CCT |
| 32 | PolyA | GACTGTGCCTTCTAGTTGCCAGCCATCTGTTGTTTGCCCCTCCCCCGT |
| | | GCCTTCCTTGACCCTGGAAGGTGCCACTCCCACTGTCCTTTCCTAATA |
| | | AAATGAGGAAATTGCATCGCATTGTCTGAGTAGGTGTCATTCTATTC |
| | | TGGGGGGTGGGGTGGGGCAGGACAGCAAGGGGGAGGATTGGGAAG |
| | | ACAATAGCAGGCATGCTGGGGATGCGGTGGGCTCTATGGC |
| 33 | Right ITR | AGGAACCCCTAGTGATGGAGTTGGCCACTCCCTCTCTGCGCGCTCGC |
| | | TCGCTCACTGAGGCCGGGCGACCAAAGGTCGCCCGACGCCCGGGCT |
| | | TTGCCCGGGCGGCCTCAGTGAGCGAGCGAGCGCGCAGCTGCCTGCA |
| | | GG |
| 34 | E2A | TTAAAAGTCGAAGGGGTTCTCGCGCTCGTCGTTGTGCGCCGCGCTGG |
| | | GGAGGGCCACGTTGCGGAACTGGTACTTGGGCTGCCACTTGAACTC |
| | | GGGGATCACCAGTTTGGGCACTGGGGTCTCGGGGAAGGTCTCGCTC |
| | | CACATGCGCCGGCTCATCTGCAGGGCGCCCAGCATGTCAGGCGCGG |
| | | AGATCTTGAAATCGCAGTTGGGGCCGGTGCTCTGCGCGCGCGAGTTG |
| | | CGGTACACTGGGTTGCAGCACTGGAACACCATCAGACTGGGGTACT |
| | | TCACACTAGCCAGCACGCTCTTGTCGCTGATCTGATCCTTGTCCAGG |
| | | TCCTCGGCGTTGCTCAGGCCGAACGGGGTCATCTTGCACAGCTGGCG |
| | | GCCCAGGAAGGGCACGCTCTGAGGCTTGTGGTTACACTCGCAGTGC |
| | | ACGGGCATCAGCATCATCCCCGCGCCGCGCTGCATATTCGGGTAGA |
| | | GGGCCTTGACGAAGGCCGCGATCTGCTTGAAAGCTTGCTGGGCCTTG |
| | | GCCCCCTCGCTGAAAAACAGGCCGCAGCTCTTCCCGCTGAACTGATT |
| | | ATTCCCGCACCCGGCATCATGGACGCAGCAGCGCGCGTCATGGCTG |
| | | GTCAGTTGCACCACGCTCCGTCCCCAGCGGTTCTGGGTCACCTTGGC |
| | | CTTGCTGGGTTGCTCCTTCAGCGCACGCTGCCCGTTCTCACTGGTCAC |
| | | ATCCATCTCCACCACGTGGTCCTTGTGGATCATCACCGTCCCATGCA |
| | | GACACTTGAGCTGGCCTTCCACCTCGGTGCAGCCGTGGTCCCACAGG |
| | | GCACTGCCGGTGCACTCCCAGTTCTTGTGCGCGATCCCGCTGTGGCT |
| | | GAAGATGTAACCTTGCAACAGGCGACCCATGATGGTGCTAAAGCTC |
| | | TTCTGGGTGGTGAAGGTCAGTTGCAGACCGCGGGCCTCCTCGTTCAT |
| | | CCAGGTCTGGCACATCTTTTGGAAGATCTCGGTCTGCTCGGGCATGA |
| | | GCTTGTAAGCATCGCGCAGGCCGCTGTCGACGCGGTAGCGTTCCATC |
| | | AGCACATTCATGGTATCCATGCCCTTCTCCCAGGACGAGACCAGAGG |
| | | CAGACTCAGGGGGTTGCGCACGTTCAGGACACCGGGGGTCGCGGGC |
| | | TCGACGATGCGTTTTCCGTCCTTGCCTTCCTTCAACAGAACCGGCGG |
| | | CTGGCTGAATCCCACTCCCACGATCACGGCTTCTTCCTGGGGCATCT |
| | | CTTCGTCTGGGTCTACCTTGGTCACATGCTTGGTCTTTCTGGCTTGCT |
| | | TCTTTTTTGGAGGGCTGTCCACGGGGACCACGTCCTCCTCGGAAGAC |
| | | CCGGATCCCACCCGCTGATACTTTCGGCGCTTGGTTGGCAGAGGAGG |
| | | TGGCGGCGAGGGGCTCCTCTCCTGCTCCGGCGGATAGCGCGCTGAA |
| | | CCGTGGCCCCGGGGCGGAGTGGCCTCTCGGTCCATGAACCGGCGCA |
| | | CGTCCTGACTGCCGCCGGCCAT |
| 35 | E4 | TCATGTATCTTTATTGATTTTTACACCAGCACGGGTAGTCAGTCTCCC |
| | | ACCACCAGCCCATTTCACAGTGTAAACAATTCTCTCAGCACGGGTGG |
| | | CCTTAAATAGGGCAATATTCTGATTAGTGCGGGAACTGGACTTGGGG |
| | | TCTATAATCCACACAGTTTCCTGGCGAGCCAAACGGGGGTCGGTGAT |
| | | TGAGATGAAGCCGTCCTCTGAAAAGTCATCCAAGCGAGCCTCACAG |
| | | TCCAAGGTCACAGTATTATGATAATCTGCATGATCACAATCGGGCAA |
| | | CAGGGGATGTTGTTCAGTCAGTGAAGCCCTGGTTTCCTCATCAGATC |
| | | GTGGTAAACGGGCCCTGCGATATGGATGATGGCGGAGCGAGCTGGA |
| | | TTGAATCTCGGTTTGCAT |
| 36 | VA RNA | AGCGGGCACTCTTCCGTGGTCTGGTGGATAAATTCGCAAGGGTATCA |
| | | TGGCGGACGACCGGGGTTCGAGCCCCGTATCCGGCCGTCCGCCGTG |
| | | ATCCATGCGGTTACCGCCCGCGTGTCGAACCCAGGTGTGCGACGTCA |
| | | GACAACGGGGGAGTGCTCCTTT |
| 37 | AAV2 Rep | ATGGCTGCCGATGGTTATCTTCCAGATTGGCTCGAGGACACTCTCTC |
| | | TGAAGGAATAAGACAGTGGTGGAAGCTCAAACCTGGCCCACCACCA |
| | | CCAAAGCCCGCAGAGCGGCATAAGGACGACAGCAGGGGTCTTGTGC |
| | | TTCCTGGGTACAAGTACCTCGGACCCTTCAACGGACTCGACAAGGG |
| | | AGAGCCGGTCAACGAGGCAGACGCCGCGGCCCTCGAGCACGACAAA |
| | | GCCTACGACCGGCAGCTCGACAGCGGAGACAACCCGTACCTCAAGT |
| | | ACAACCACGCCGACGCGGAGTTTCAGGAGCGCCTTAAAGAAGATAC |
| | | GTCTTTTGGGGGCAACCTCGGACGAGCAGTCTTCCAGGCGAAAAAG |
| | | AGGGTTCTTGAACCTCTGGGCCTGGTTGAGGAACCTGTTAAGACGGC |
| | | TCCGGGAAAAAAGAGGCCGGTAGAGCACTCTCCTGTGGAGCCAGAC |
| | | TCCTCCTCGGGAACCGGAAAGGCGGGCCAGCAGCCTGCAAGAAAAA |
| | | GATTGAATTTTGGTCAGACTGGAGACGCAGACTCAGTACCTGACCCC |
| | | CAGCCTCTCGGACAGCCACCAGCAGCCCCCTCTGGTCTGGGAACTAA |
| | | TACGATGGCTACAGGCAGTGGCGCACCAATGGCAGACAATAACGAG |
| | | GGCGCCGACGGAGTGGGTAATTCCTCGGGAAATTGGCATTGCGATT |
| | | CCACATGGATGGGCGACAGAGTCATCACCACCAGCACCCGAACCTG |
| | | GGCCCTGCCCACCTACAACAACCACCTCTACAAACAAATTTCCAGCC |
| | | AATCAGGAGCCTCGAACGACAATCACTACTTTGGCTACAGCACCCCT |
| | | TGGGGGTATTTTGACTTCAACAGATTCCACTGCCACTTTTCACCACG |
| | | TGACTGGCAAAGACTCATCAACAACAACTGGGGATTCCGACCCAAG |
| | | AGACTCAACTTCAAGCTCTTTAACATTCAAGTCAAAGAGGTCACGCA |
| | | GAATGACGGTACGACGACGATTGCCAATAACCTTACCAGCACGGTT |
| | | CAGGTGTTTACTGACTCGGAGTACCAGCTCCCGTACGTCCTCGGCTC |
| | | GGCGCATCAAGGATGCCTCCCGCCGTTCCCAGCAGACGTCTTCATGG |
| | | TGCCACAGTATGGATACCTCACCCTGAACAACGGGAGTCAGGCAGT |
| | | AGGACGCTCTTCATTTTACTGCCTGGAGTACTTTCCTTCTCAGATGCT |
| | | GCGTACCGGAAACAACTTTACCTTCAGCTACACTTTTGAGGACGTTC |
| | | CTTTCCACAGCAGCTACGCTCACAGCCAGAGTCTGGACCGTCTCATG |
| | | AATCCTCTCATCGACCAGTACCTGTATTACTTGAGCAGAACAAACAC |
| | | TCCAAGTGGAACCACCACGCAGTCAAGGCTTCAGTTTTCTCAGGCCG |
| | | GAGCGAGTGACATTCGGGACCAGTCTAGGAACTGGCTTCCTGGACC |
| | | CTGTTACCGCCAGCAGCGAGTATCAAAGACATCTGCGGATAACAAC |
| | | AACAGTGAATACTCGTGGACTGGAGCTACCAAGTACCACCTCAATG |
| | | GCAGAGACTCTCTGGTGAATCCGGGCCCGGCCATGGCAAGCCACAA |
| | | GGACGATGAAGAAAAGTTTTTTCCTCAGAGCGGGGTTCTCATCTTTG |
| | | GGAAGCAAGGCTCAGAGAAAACAAATGTGGACATTGAAAAGGTCAT |
| | | GATTACAGACGAAGAGGAAATCAGGACAACCAATCCCGTGGCTACG |
| | | GAGCAGTATGGTTCTGTATCTACCAACCTCCAGAGAGGCAACAGAC |
| | | AAGCAGCTACCGCAGATGTCAACACACAAGGCGTTCTTCCAGGCAT |
| | | GGTCTGGCAGGACAGAGATGTGTACCTTCAGGGGCCCATCTGGGCA |
| | | AAGATTCCACACACGGACGGACATTTTCACCCCTCTCCCCTCATGGG |
| | | TGGATTCGGACTTAAACACCCTCCTCCACAGATTCTCATCAAGAACA |
| | | CCCCGGTACCTGCGAATCCTTCGACCACCTTCAGTGCGGCAAAGTTT |
| | | GCTTCCTTCATCACACAGTACTCCACGGGACAGGTCAGCGTGGAGAT |
| | | CGAGTGGGAGCTGCAGAAGGAAAACAGCAAACGCTGGAATCCCGA |
| | | AATTCAGTACACTTCCAACTACAACAAGTCTGTTAATGTGGACTTTA |
| | | CTGTGGACACTAATGGCGTGTATTCAGAGCCTCGCCCCATTGGCACC |
| | | AGATACCTGACTCGTAATCTGTAA |
| 38 | AAV2 Cap | ATGCCGGGGTTTTACGAGATTGTGATTAAGGTCCCCAGCGACCTTGA |
| | | CGAGCATCTGCCCGGCATTTCTGACAGCTTTGTGAACTGGGTGGCCG |
| | | AGAAGGAATGGGAGTTGCCGCCAGATTCTGACATGGATCTGAATCT |
| | | GATTGAGCAGGCACCCCTGACCGTGGCCGAGAAGCTGCAGCGCGAC |
| | | TTTCTGACGGAATGGCGCCGTGTGAGTAAGGCCCCGGAGGCCCTTTT |
| | | CTTTGTGCAATTTGAGAAGGGAGAGAGCTACTTCCACATGCACGTGC |
| | | TCGTGGAAACCACCGGGGTGAAATCCATGGTTTTGGGACGTTTCCTG |
| | | AGTCAGATTCGCGAAAAACTGATTCAGAGAATTTACCGCGGGATCG |
| | | AGCCGACTTTGCCAAACTGGTTCGCGGTCACAAAGACCAGAAATGG |
| | | CGCCGGAGGCGGGAACAAGGTGGTGGATGAGTGCTACATCCCCAAT |
| | | TACTTGCTCCCCAAAACCCAGCCTGAGCTCCAGTGGGCGTGGACTAA |
| | | TATGGAACAGTATTTAAGCGCCTGTTTGAATCTCACGGAGCGTAAAC |
| | | GGTTGGTGGCGCAGCATCTGACGCACGTGTCGCAGACGCAGGAGCA |
| | | GAACAAAGAGAATCAGAATCCCAATTCTGATGCGCCGGTGATCAGA |
| | | TCAAAAACTTCAGCCAGGTACATGGAGCTGGTCGGGTGGCTCGTGG |
| | | ACAAGGGGATTACCTCGGAGAAGCAGTGGATCCAGGAGGACCAGGC |
| | | CTCATACATCTCCTTCAATGCGGCCTCCAACTCGCGGTCCCAAATCA |
| | | AGGCTGCCTTGGACAATGCGGGAAAGATTATGAGCCTGACTAAAAC |
| | | CGCCCCCGACTACCTGGTGGGCCAGCAGCCCGTGGAGGACATTTCC |
| | | AGCAATCGGATTTATAAAATTTTGGAACTAAACGGGTACGATCCCCA |
| | | ATATGCGGCTTCCGTCTTTCTGGGATGGGCCACGAAAAAGTTCGGCA |
| | | AGAGGAACACCATCTGGCTGTTTGGGCCTGCAACTACCGGGAAGAC |
| | | CAACATCGCGGAGGCCATAGCCCACACTGTGCCCTTCTACGGGTGCG |
| | | TAAACTGGACCAATGAGAACTTTCCCTTCAACGACTGTGTCGACAAG |
| | | ATGGTGATCTGGTGGGAGGAGGGGAAGATGACCGCCAAGGTCGTGG |
| | | AGTCGGCCAAAGCCATTCTCGGAGGAAGCAAGGTGCGCGTGGACCA |
| | | GAAATGCAAGTCCTCGGCCCAGATAGACCCGACTCCCGTGATCGTC |
| | | ACCTCCAACACCAACATGTGCGCCGTGATTGACGGGAACTCAACGA |
| | | CCTTCGAACACCAGCAGCCGTTGCAAGACCGGATGTTCAAATTTGAA |
| | | CTCACCCGCCGTCTGGATCATGACTTTGGGAAGGTCACCAAGCAGG |
| | | AAGTCAAAGACTTTTTCCGGTGGGCAAAGGATCACGTGGTTGAGGT |
| | | GGAGCATGAATTCTACGTCAAAAAGGGTGGAGCCAAGAAAAGACCC |
| | | GCCCCCAGTGACGCAGATATAAGTGAGCCCAAACGGGTGCGCGAGT |
| | | CAGTTGCGCAGCCATCGACGTCAGACGCGGAAGCTTCGATCAACTA |
| | | CGCAGACAGGTACCAAAACAAATGTTCTCGTCACGTGGGCATGAAT |
| | | CTGATGCTGTTTCCCTGCAGACAATGCGAGAGAATGAATCAGAATTC |
| | | AAATATCTGCTTCACTCACGGACAGAAAGACTGTTTAGAGTGCTTTC |
| | | CCGTGTCAGAATCTCAACCCGTTTCTGTCGTCAAAAAGGCGTATCAG |
| | | AAACTGTGCTACATTCATCATATCATGGGAAAGGTGCCAGACGCTTG |
| | | CACTGCCTGCGATCTGGTCAATGTGGATTTGGATGACTGCATCTTTG |
| | | AACAATAA |
| 39 | AAV8 Cap | ATGGCTGCAGGCGGTGGCGCACCAATGGCAGACAATAACGAAGGCG |
| | | CCGACGGAGTGGGTAGTTCCTCGGGAAATTGGCATTGCGATTCCACA |
| | | TGGCTGGGCGACAGAGTCATCACCACCAGCACCCGAACCTGGGCCC |
| | | TGCCCACCTACAACAACCACCTCTACAAGCAAATCTCCAACGGGAC |
| | | ATCGGGAGGAGCCACCAACGACAACACCTACTTCGGCTACAGCACC |
| | | CCCTGGGGGTATTTTGACTTTAACAGATTCCACTGCCACTTTTCACCA |
| | | CGTGACTGGCAGCGACTCATCAACAACAACTGGGGATTCCGGCCCA |
| | | AGAGACTCAGCTTCAAGCTCTTCAACATCCAGGTCAAGGAGGTCAC |
| | | GCAGAATGAAGGCACCAAGACCATCGCCAATAACCTCACCAGCACC |
| | | ATCCAGGTGTTTACGGACTCGGAGTACCAGCTGCCGTACGTTCTCGG |
| | | CTCTGCCCACCAGGGCTGCCTGCCTCCGTTCCCGGCGGACGTGTTCA |
| | | TGATTCCCCAGTACGGCTACCTAACACTCAACAACGGTAGTCAGGCC |
| | | GTGGGACGCTCCTCCTTCTACTGCCTGGAATACTTTCCTTCGCAGAT |
| | | GCTGAGAACCGGCAACAACTTCCAGTTTACTTACACCTTCGAGGACG |
| | | TGCCTTTCCACAGCAGCTACGCCCACAGCCAGAGCTTGGACCGGCTG |
| | | ATGAATCCTCTGATTGACCAGTACCTGTACTACTTGTCTCGGACTCA |
| | | AACAACAGGAGGCACGGCAAATACGCAGACTCTGGGCTTCAGCCAA |
| | | GGTGGGCCTAATACAATGGCCAATCAGGCAAAGAACTGGCTGCCAG |
| | | GACCCTGTTACCGCCAACAACGCGTCTCAACGACAACCGGGCAAAA |
| | | CAACAATAGCAACTTTGCCTGGACTGCTGGGACCAAATACCATCTGA |
| | | ATGGAAGAAATTCATTGGCTAATCCTGGCATCGCTATGGCAACACAC |
| | | AAAGACGACGAGGAGCGTTTTTTTCCCAGTAACGGGATCCTGATTTT |
| | | TGGCAAACAAAATGCTGCCAGAGACAATGCGGATTACAGCGATGTC |
| | | ATGCTCACCAGCGAGGAAGAAATCAAAACCACTAACCCTGTGGCTA |
| | | CAGAGGAATACGGTATCGTGGCAGATAACTTGCAGCAGCAAAACAC |
| | | GGCTCCTCAAATTGGAACTGTCAACAGCCAGGGGGCCTTACCCGGT |
| | | ATGGTCTGGCAGAACCGGGACGTGTACCTGCAGGGTCCCATCTGGG |
| | | CCAAGATTCCTCACACGGACGGCAACTTCCACCCGTCTCCGCTGATG |
| | | GGCGGCTTTGGCCTGAAACATCCTCCGCCTCAGATCCTGATCAAGAA |
| | | CACGCCTGTACCTGCGGATCCTCCGACCACCTTCAACCAGTCAAAGC |
| | | TGAACTCTTTCATCACGCAATACAGCACCGGACAGGTCAGCGTGGA |
| | | AATTGAATGGGAGCTGCAGAAGGAAAACAGCAAGCGCTGGAACCCC |
| | | GAGATCCAGTACACCTCCAACTACTACAAATCTACAAGTGTGGACTT |
| | | TGCTGTTAATACAGAAGGCGTGTACTCTGAACCCCGCCCCATTGGCA |
| | | CCCGTTACCTCACCCGTAATCTGTAA |
| 40 | AAV DJ Cap | ATGGCTGCCGATGGTTATCTTCCAGATTGGCTCGAGGACACTCTCTC |
| | | TGAAGGAATAAGACAGTGGTGGAAGCTCAAACCTGGCCCACCACCA |
| | | CCAAAGCCCGCAGAGCGGCATAAGGACGACAGCAGGGGTCTTGTGC |
| | | TTCCTGGGTACAAGTACCTCGGACCCTTCAACGGACTCGACAAGGG |
| | | AGAGCCGGTCAACGAGGCAGACGCCGCGGCCCTCGAGCACGACAAA |
| | | GCCTACGACCGGCAGCTCGACAGCGGAGACAACCCGTACCTCAAGT |
| | | ACAACCACGCCGACGCCGAGTTCCAGGAGCGGCTCAAAGAAGATAC |
| | | GTCTTTTGGGGGCAACCTCGGGCGAGCAGTCTTCCAGGCCAAAAAG |
| | | AGGCTTCTTGAACCTCTTGGTCTGGTTGAGGAAGCGGCTAAGACGGC |
| | | TCCTGGAAAGAAGAGGCCTGTAGAGCACTCTCCTGTGGAGCCAGAC |
| | | TCCTCCTCGGGAACCGGAAAGGCGGGCCAGCAGCCTGCAAGAAAAA |
| | | GATTGAATTTTGGTCAGACTGGAGACGCAGACTCAGTCCCAGACCCT |
| | | CAACCAATCGGAGAACCTCCCGCAGCCCCCTCAGGTGTGGGATCTCT |
| | | TACAATGGCTGCAGGCGGTGGCGCACCAATGGCAGACAATAACGAG |
| | | GGCGCCGACGGAGTGGGTAATTCCTCGGGAAATTGGCATTGCGATT |
| | | CCACATGGATGGGCGACAGAGTCATCACCACCAGCACCCGAACCTG |
| | | GGCCCTGCCCACCTACAACAACCACCTCTACAAGCAAATCTCCAACA |
| | | GCACATCTGGAGGATCTTCAAATGACAACGCCTACTTCGGCTACAGC |
| | | ACCCCCTGGGGGTATTTTGACTTTAACAGATTCCACTGCCACTTTTCA |
| | | CCACGTGACTGGCAGCGACTCATCAACAACAACTGGGGATTCCGGC |
| | | CCAAGAGACTCAGCTTCAAGCTCTTCAACATCCAGGTCAAGGAGGT |
| | | CACGCAGAATGAAGGCACCAAGACCATCGCCAATAACCTCACCAGC |
| | | ACCATCCAGGTGTTTACGGACTCGGAGTACCAGCTGCCGTACGTTCT |
| | | CGGCTCTGCCCACCAGGGCTGCCTGCCTCCGTTCCCGGCGGACGTGT |
| | | TCATGATTCCCCAGTACGGCTACCTAACACTCAACAACGGTAGTCAG |
| | | GCCGTGGGACGCTCCTCCTTCTACTGCCTGGAATACTTTCCTTCGCA |
| | | GATGCTGAGAACCGGCAACAACTTCCAGTTTACTTACACCTTCGAGG |
| | | ACGTGCCTTTCCACAGCAGCTACGCCCACAGCCAGAGCTTGGACCG |
| | | GCTGATGAATCCTCTGATTGACCAGTACCTGTACTACTTGTCTCGGA |
| | | CTCAAACAACAGGAGGCACGACAAATACGCAGACTCTGGGCTTCAG |
| | | CCAAGGTGGGCCTAATACAATGGCCAATCAGGCAAAGAACTGGCTG |
| | | CCAGGACCCTGTTACCGCCAGCAGCGAGTATCAAAGACATCTGCGG |
| | | ATAACAACAACAGTGAATACTCGTGGACTGGAGCTACCAAGTACCA |
| | | CCTCAATGGCAGAGACTCTCTGGTGAATCCGGGCCCGGCCATGGCA |
| | | AGCCACAAGGACGATGAAGAAAAGTTTTTTCCTCAGAGCGGGGTTC |
| | | TCATCTTTGGGAAGCAAGGCTCAGAGAAAACAAATGTGGACATTGA |
| | | AAAGGTCATGATTACAGACGAAGAGGAAATCAGGACAACCAATCCC |
| | | GTGGCTACGGAGCAGTATGGTTCTGTATCTACCAACCTCCAGAGAGG |
| | | CAACAGACAAGCAGCTACCGCAGATGTCAACACACAAGGCGTTCTT |
| | | CCAGGCATGGTCTGGCAGGACAGAGATGTGTACCTTCAGGGGCCCA |
| | | TCTGGGCAAAGATTCCACACACGGACGGACATTTTCACCCCTCTCCC |
| | | CTCATGGGTGGATTCGGACTTAAACACCCTCCGCCTCAGATCCTGAT |
| | | CAAGAACACGCCTGTACCTGCGGATCCTCCGACCACCTTCAACCAGT |
| | | CAAAGCTGAACTCTTTCATCACCCAGTATTCTACTGGCCAAGTCAGC |
| | | GTGGAGATCGAGTGGGAGCTGCAGAAGGAAAACAGCAAGCGCTGG |
| | | AACCCCGAGATCCAGTACACCTCCAACTACTACAAATCTACAAGTGT |
| | | GGACTTTGCTGTTAATACAGAAGGCGTGTACTCTGAACCCCGCCCCA |
| | | TTGGCACCCGTTACCTCACCCGTAATCTGTAA |
| 41 | Chicken beta | GGAGTCGCTGCGTTGCCTTCGCCCCGTGCCCCGCTCCGCGCCGCCTC |
| | actin intron | GCGCCGCCCGCCCCGGCTCTGACTGACCGCGTTACTCCCACAGGTGA |
| | | GCGGGCGGGACGGCCCTTCTCCTCCGGGCTGTAATTAGCGCTTGGTT |
| | | TAATGACGGCTCGTTTCTTTTCTGTGGCTGCGTGAAAGCCTTAAAGG |
| | | GCTCCGGGAGGGCCCTTTGTGCGGGGGGGAGCGGCTCGGGGGGTGC |
| | | GTGCGTGTGTGTGTGCGTGGGGAGCGCCGCGTGCGGCCCGCGCTGC |
| | | CCGGCGGCTGTGAGCGCTGCGGGCGCGGCGCGGGGCTTTGTGCGCT |
| | | CCGCGTGTGCGCGAGGGGAGCGCGGCCGGGGGCGGTGCCCCGCGGT |
| | | GCGGGGGGGCTGCGAGGGGAACAAAGGCTGCGTGCGGGGTGTGTGC |
| | | GTGGGGGGGTGAGCAGGGGGTGTGGGCGCGGCGGTCGGGCTGTAAC |
| | | CCCCCCCTGCACCCCCCTCCCCGAGTTGCTGAGCACGGCCCGGCTTC |
| | | GGGTGCGGGGCTCCGTGCGGGGCGTGGCGCGGGGCTCGCCGTGCCG |
| | | GGCGGGGGGTGGCGGCAGGTGGGGGTGCCGGGCGGGGCGGGGCCG |
| | | CCTCGGGCCGGGGAGGGCTCGGGGGAGGGGCGCGGCGGCCCCGGA |
| | | GCGCCGGCGGCTGTCGAGGCGCGGCGAGCCGCAGCCATTGCCTTTT |
| | | ATGGTAATCGTGCGAGAGGGCGCAGGGACTTCCTTTGTCCCAAATCT |
| | | GGCGGAGCCGAAATCTGGGAGGCGCCGCCGCACCCCCTCTAGCGGG |
| | | CGCGGGCGAAGCGGTGCGGCGCCGGCAGGAAGGAAATGGGCGGGG |
| | | AGGGCCTTCGTGCGTCGCCGCGCCGCCGTCCCCTTCTCCATCTCCAG |
| | | CCTCGGGGCTGCCGCAGGGGGACGGCTGCCTTCGGGGGGGACGGGG |
| | | CAGGGCGGGGTTCGGCTTCTGGCGTGTGACCGGCGG |
| 42 | Rabbit beta | AGATCTTTTTCCCTCTGCCAAAAATTATGGGGACATCATGAAGCCCC |
| | globin poly A | TTGAGCATCTGACTTCTGGCTAATAAAGGAAATTTATTTTCATTGCA |
| | | ATAGTGTGTTGGAATTTTTTGTGTCTCTCACTCGGAAGGACATATGG |
| | | GAGGGCAAATCATTTAAAACATCAGAATGAGTATTTGGTTTAGAGTT |
| | | TGGCAACATATGCCATATGCTGGCTGCCATGAACAAAGGTGGCTAT |
| | | AAAGAGGTCATCAGTATATGAAACAGCCCCCTGCTGTCCATTCCTTA |
| | | TTCCATAGAAAAGCCTTGACTTGAGGTTAGATTTTTTTTATATTTTGT |
| | | TTTGTGTTATTTTTTTCTTTAACATCCCTAAAATTTTCCTTACATGTTT |
| | | TACTAGCCAGATTTTTCCTCCTCTCCTGACTACTCCCAGTCATAGCTG |
| | | TCCCTCTTCTCTTATGAAGATC |
| 43 | Primer | TAAGCAGAATTCATGAATTTGCCAGGAAGAT |
| 44 | Primer | CCATACAATGAATGGACACTAGGCGGCCGCACGAAT |
| 45 | Gag, Pol, | GAATTCATGAATTTGCCAGGAAGATGGAAACCAAAAATGATAGGGG |
| | Integrase | GAATTGGAGGTTTTATCAAAGTAAGACAGTATGATCAGATACTCATA |
| | fragment | GAAATCTGCGGACATAAAGCTATAGGTACAGTATTAGTAGGACCTA |
| | | CACCTGTCAACATAATTGGAAGAAATCTGTTGACTCAGATTGGCTGC |
| | | ACTTTAAATTTTCCCATTAGTCCTATTGAGACTGTACCAGTAAAATT |
| | | AAAGCCAGGAATGGATGGCCCAAAAGTTAAACAATGGCCATTGACA |
| | | GAAGAAAAAATAAAAGCATTAGTAGAAATTTGTACAGAAATGGAAA |
| | | AGGAAGGAAAAATTTCAAAAATTGGGCCTGAAAATCCATACAATAC |
| | | TCCAGTATTTGCCATAAAGAAAAAAGACAGTACTAAATGGAGAAAA |
| | | TTAGTAGATTTCAGAGAACTTAATAAGAGAACTCAAGATTTCTGGGA |
| | | AGTTCAATTAGGAATACCACATCCTGCAGGGTTAAAACAGAAAAAA |
| | | TCAGTAACAGTACTGGATGTGGGCGATGCATATTTTTCAGTTCCCTT |
| | | AGATAAAGACTTCAGGAAGTATACTGCATTTACCATACCTAGTATAA |
| | | ACAATGAGACACCAGGGATTAGATATCAGTACAATGTGCTTCCACA |
| | | GGGATGGAAAGGATCACCAGCAATATTCCAGTGTAGCATGACAAAA |
| | | ATCTTAGAGCCTTTTAGAAAACAAAATCCAGACATAGTCATCTATCA |
| | | ATACATGGATGATTTGTATGTAGGATCTGACTTAGAAATAGGGCAGC |
| | | ATAGAACAAAAATAGAGGAACTGAGACAACATCTGTTGAGGTGGGG |
| | | ATTTACCACACCAGACAAAAAACATCAGAAAGAACCTCCATTCCTTT |
| | | GGATGGGTTATGAACTCCATCCTGATAAATGGACAGTACAGCCTATA |
| | | GTGCTGCCAGAAAAGGACAGCTGGACTGTCAATGACATACAGAAAT |
| | | TAGTGGGAAAATTGAATTGGGCAAGTCAGATTTATGCAGGGATTAA |
| | | AGTAAGGCAATTATGTAAACTTCTTAGGGGAACCAAAGCACTAACA |
| | | GAAGTAGTACCACTAACAGAAGAAGCAGAGCTAGAACTGGCAGAA |
| | | AACAGGGAGATTCTAAAAGAACCGGTACATGGAGTGTATTATGACC |
| | | CATCAAAAGACTTAATAGCAGAAATACAGAAGCAGGGGCAAGGCC |
| | | AATGGACATATCAAATTTATCAAGAGCCATTTAAAAATCTGAAAAC |
| | | AGGAAAGTATGCAAGAATGAAGGGTGCCCACACTAATGATGTGAAA |
| | | CAATTAACAGAGGCAGTACAAAAAATAGCCACAGAAAGCATAGTAA |
| | | TATGGGGAAAGACTCCTAAATTTAAATTACCCATACAAAAGGAAAC |
| | | ATGGGAAGCATGGTGGACAGAGTATTGGCAAGCCACCTGGATTCCT |
| | | GAGTGGGAGTTTGTCAATACCCCTCCCTTAGTGAAGTTATGGTACCA |
| | | GTTAGAGAAAGAACCCATAATAGGAGCAGAAACTTTCTATGTAGAT |
| | | GGGGCAGCCAATAGGGAAACTAAATTAGGAAAAGCAGGATATGTA |
| | | ACTGACAGAGGAAGACAAAAAGTTGTCCCCCTAACGGACACAACAA |
| | | ATCAGAAGACTGAGTTACAAGCAATTCATCTAGCTTTGCAGGATTCG |
| | | GGATTAGAAGTAAACATAGTGACAGACTCACAATATGCATTGGGAA |
| | | TCATTCAAGCACAACCAGATAAGAGTGAATCAGAGTTAGTCAGTCA |
| | | AATAATAGAGCAGTTAATAAAAAAGGAAAAAGTCTACCTGGCATGG |
| | | GTACCAGCACACAAAGGAATTGGAGGAAATGAACAAGTAGATAAAT |
| | | TGGTCAGTGCTGGAATCAGGAAAGTACTATTTTTAGATGGAATAGAT |
| | | AAGGCCCAAGAAGAACATGAGAAATATCACAGTAATTGGAGAGCA |
| | | ATGGCTAGTGATTTTAACCTACCACCTGTAGTAGCAAAAGAAATAGT |
| | | AGCCAGCTGTGATAAATGTCAGCTAAAAGGGGAAGCCATGCATGGA |
| | | CAAGTAGACTGTAGCCCAGGAATATGGCAGCTAGATTGTACACATTT |
| | | AGAAGGAAAAGTTATCTTGGTAGCAGTTCATGTAGCCAGTGGATAT |
| | | ATAGAAGCAGAAGTAATTCCAGCAGAGACAGGGCAAGAAACAGCA |
| | | TACTTCCTCTTAAAATTAGCAGGAAGATGGCCAGTAAAAACAGTAC |
| | | ATACAGACAATGGCAGCAATTTCACCAGTACTACAGTTAAGGCCGC |
| | | CTGTTGGTGGGCGGGGATCAAGCAGGAATTTGGCATTCCCTACAATC |
| | | CCCAAAGTCAAGGAGTAATAGAATCTATGAATAAAGAATTAAAGAA |
| | | AATTATAGGACAGGTAAGAGATCAGGCTGAACATCTTAAGACAGCA |
| | | GTACAAATGGCAGTATTCATCCACAATTTTAAAAGAAAAGGGGGGA |
| | | TTGGGGGGTACAGTGCAGGGGAAAGAATAGTAGACATAATAGCAAC |
| | | AGACATACAAACTAAAGAATTACAAAAACAAATTACAAAAATTCAA |
| | | AATTTTCGGGTTTATTACAGGGACAGCAGAGATCCAGTTTGGAAAG |
| | | GACCAGCAAAGCTCCTCTGGAAAGGTGAAGGGGCAGTAGTAATACA |
| | | AGATAATAGTGACATAAAAGTAGTGCCAAGAAGAAAAGCAAAGAT |
| | | CATCAGGGATTATGGAAAACAGATGGCAGGTGATGATTGTGTGGCA |
| | | AGTAGACAGGATGAGGATTAA |
| 46 | DNA | TCTAGAATGGCAGGAAGAAGCGGAGACAGCGACGAAGAGCTCATC |
| | Fragment | AGAACAGTCAGACTCATCAAGCTTCTCTATCAAAGCAACCCACCTCC |
| | | CAATCCCGAGGGGACCCGACAGGCCCGAAGGAATAGAAGAAGAAG |
| | | GTGGAGAGAGAGACAGAGACAGATCCATTCGATTAGTGAACGGATC |
| | | CTTGGCACTTATCTGGGACGATCTGCGGAGCCTGTGCCTCTTCAGCT |
| | | ACCACCGCTTGAGAGACTTACTCTTGATTGTAACGAGGATTGTGGAA |
| | | CTTCTGGGACGCAGGGGGTGGGAAGCCCTCAAATATTGGTGGAATC |
| | | TCCTACAATATTGGAGTCAGGAGCTAAAGAATAGAGGAGCTTTGTTC |
| | | CTTGGGTTCTTGGGAGCAGCAGGAAGCACTATGGGCGCAGCGTCAA |
| | | TGACGCTGACGGTACAGGCCAGACAATTATTGTCTGGTATAGTGCAG |
| | | CAGCAGAACAATTTGCTGAGGGCTATTGAGGCGCAACAGCATCTGT |
| | | TGCAACTCACAGTCTGGGGCATCAAGCAGCTCCAGGCAAGAATCCT |
| | | GGCTGTGGAAAGATACCTAAAGGATCAACAGCTCCTAGATCTTTTTC |
| | | CCTCTGCCAAAAATTATGGGGACATCATGAAGCCCCTTGAGCATCTG |
| | | ACTTCTGGCTAATAAAGGAAATTTATTTTCATTGCAATAGTGTGTTG |
| | | GAATTTTTTGTGTCTCTCACTCGGAAGGACATATGGGAGGGCAAATC |
| | | ATTTAAAACATCAGAATGAGTATTTGGTTTAGAGTTTGGCAACATAT |
| | | GCCATATGCTGGCTGCCATGAACAAAGGTGGCTATAAAGAGGTCAT |
| | | CAGTATATGAAACAGCCCCCTGCTGTCCATTCCTTATTCCATAGAAA |
| | | AGCCTTGACTTGAGGTTAGATTTTTTTTATATTTTGTTTTGTGTTATTT |
| | | TTTTCTTTAACATCCCTAAAATTTTCCTTACATGTTTTACTAGCCAGA |
| | | TTTTTCCTCCTCTCCTGACTACTCCCAGTCATAGCTGTCCCTCTTCTCT |
| | | TATGAAGATCCCTCGACCTGCAGCCCAAGCTTGGCGTAATCATGGTC |
| | | ATAGCTGTTTCCTGTGTGAAATTGTTATCCGCTCACAATTCCACACA |
| | | ACATACGAGCCGGAAGCATAAAGTGTAAAGCCTGGGGTGCCTAATG |
| | | AGTGAGCTAACTCACATTAATTGCGTTGCGCTCACTGCCCGCTTTCC |
| | | AGTCGGGAAACCTGTCGTGCCAGCGGATCCGCATCTCAATTAGTCAG |
| | | CAACCATAGTCCCGCCCCTAACTCCGCCCATCCCGCCCCTAACTCCG |
| | | CCCAGTTCCGCCCATTCTCCGCCCCATGGCTGACTAATTTTTTTTATT |
| | | TATGCAGAGGCCGAGGCCGCCTCGGCCTCTGAGCTATTCCAGAAGT |
| | | AGTGAGGAGGCTTTTTTGGAGGCCTAGGCTTTTGCAAAAAGCTAACT |
| | | TGTTTATTGCAGCTTATAATGGTTACAAATAAAGCAATAGCATCACA |
| | | AATTTCACAAATAAAGCATTTTTTTCACTGCATTCTAGTTGTGGTTTG |
| | | TCCAAACTCATCAATGTATCTTATCAGCGGCCGCCCCGGG |
| 47 | DNA | ACGCGTTAGTTATTAATAGTAATCAATTACGGGGTCATTAGTTCATA |
| | fragment | GCCCATATATGGAGTTCCGCGTTACATAACTTACGGTAAATGGCCCG |
| | | CCTGGCTGACCGCCCAACGACCCCCGCCCATTGACGTCAATAATGAC |
| | | GTATGTTCCCATAGTAACGCCAATAGGGACTTTCCATTGACGTCAAT |
| | | GGGTGGACTATTTACGGTAAACTGCCCACTTGGCAGTACATCAAGTG |
| | | TATCATATGCCAAGTACGCCCCCTATTGACGTCAATGACGGTAAATG |
| | | GCCCGCCTGGCATTATGCCCAGTACATGACCTTATGGGACTTTCCTA |
| | | CTTGGCAGTACATCTACGTATTAGTCATCGCTATTACCATGGGTCGA |
| | | GGTGAGCCCCACGTTCTGCTTCACTCTCCCCATCTCCCCCCCCTCCCC |
| | | ACCCCCAATTTTGTATTTATTTATTTTTTAATTATTTTGTGCAGCGAT |
| | | GGGGGCGGGGGGGGGGGGGGCGCGCGCCAGGCGGGGCGGGGCGGG |
| | | GCGAGGGGCGGGGCGGGGCGAGGCGGAGAGGTGCGGCGGCAGCCA |
| | | ATCAGAGCGGCGCGCTCCGAAAGTTTCCTTTTATGGCGAGGCGGCG |
| | | GCGGCGGCGGCCCTATAAAAAGCGAAGCGCGCGGCGGGCGGGAGT |
| | | CGCTGCGTTGCCTTCGCCCCGTGCCCCGCTCCGCGCCGCCTCGCGCC |
| | | GCCCGCCCCGGCTCTGACTGACCGCGTTACTCCCACAGGTGAGCGGG |
| | | CGGGACGGCCCTTCTCCTCCGGGCTGTAATTAGCGCTTGGTTTAATG |
| | | ACGGCTCGTTTCTTTTCTGTGGCTGCGTGAAAGCCTTAAAGGGCTCC |
| | | GGGAGGGCCCTTTGTGCGGGGGGGAGCGGCTCGGGGGGTGCGTGCG |
| | | TGTGTGTGTGCGTGGGGAGCGCCGCGTGCGGCCCGCGCTGCCCGGC |
| | | GGCTGTGAGCGCTGCGGGCGCGGCGCGGGGCTTTGTGCGCTCCGCG |
| | | TGTGCGCGAGGGGAGCGCGGCCGGGGGCGGTGCCCCGCGGTGCGGG |
| | | GGGGCTGCGAGGGGAACAAAGGCTGCGTGCGGGGTGTGTGCGTGGG |
| | | GGGGTGAGCAGGGGGTGTGGGCGCGGCGGTCGGGCTGTAACCCCCC |
| | | CCTGCACCCCCCTCCCCGAGTTGCTGAGCACGGCCCGGCTTCGGGTG |
| | | CGGGGCTCCGTGCGGGGCGTGGCGCGGGGCTCGCCGTGCCGGGCGG |
| | | GGGGTGGCGGCAGGTGGGGGTGCCGGGCGGGGCGGGGCCGCCTCG |
| | | GGCCGGGGAGGGCTCGGGGGAGGGGCGCGGCGGCCCCGGAGCGCC |
| | | GGCGGCTGTCGAGGCGCGGCGAGCCGCAGCCATTGCCTTTTATGGTA |
| | | ATCGTGCGAGAGGGCGCAGGGACTTCCTTTGTCCCAAATCTGGCGG |
| | | AGCCGAAATCTGGGAGGCGCCGCCGCACCCCCTCTAGCGGGCGCGG |
| | | GCGAAGCGGTGCGGCGCCGGCAGGAAGGAAATGGGCGGGGAGGGC |
| | | CTTCGTGCGTCGCCGCGCCGCCGTCCCCTTCTCCATCTCCAGCCTCGG |
| | | GGCTGCCGCAGGGGGACGGCTGCCTTCGGGGGGGACGGGGCAGGGC |
| | | GGGGTTCGGCTTCTGGCGTGTGACCGGCGGGAATTC |
| 48 | RSV promoter | CAATTGCGATGTACGGGCCAGATATACGCGTATCTGAGGGGACTAG |
| | and HIV Rev | GGTGTGTTTAGGCGAAAAGCGGGGCTTCGGTTGTACGCGGTTAGGA |
| | | GTCCCCTCAGGATATAGTAGTTTCGCTTTTGCATAGGGAGGGGGAAA |
| | | TGTAGTCTTATGCAATACACTTGTAGTCTTGCAACATGGTAACGATG |
| | | AGTTAGCAACATGCCTTACAAGGAGAGAAAAAGCACCGTGCATGCC |
| | | GATTGGTGGAAGTAAGGTGGTACGATCGTGCCTTATTAGGAAGGCA |
| | | ACAGACAGGTCTGACATGGATTGGACGAACCACTGAATTCCGCATT |
| | | GCAGAGATAATTGTATTTAAGTGCCTAGCTCGATACAATAAACGCCA |
| | | TTTGACCATTCACCACATTGGTGTGCACCTCCAAGCTCGAGCTCGTT |
| | | TAGTGAACCGTCAGATCGCCTGGAGACGCCATCCACGCTGTTTTGAC |
| | | CTCCATAGAAGACACCGGGACCGATCCAGCCTCCCCTCGAAGCTAG |
| | | CGATTAGGCATCTCCTATGGCAGGAAGAAGCGGAGACAGCGACGAA |
| | | GAACTCCTCAAGGCAGTCAGACTCATCAAGTTTCTCTATCAAAGCAA |
| | | CCCACCTCCCAATCCCGAGGGGACCCGACAGGCCCGAAGGAATAGA |
| | | AGAAGAAGGTGGAGAGAGAGACAGAGACAGATCCATTCGATTAGT |
| | | GAACGGATCCTTAGCACTTATCTGGGACGATCTGCGGAGCCTGTGCC |
| | | TCTTCAGCTACCACCGCTTGAGAGACTTACTCTTGATTGTAACGAGG |
| | | ATTGTGGAACTTCTGGGACGCAGGGGGTGGGAAGCCCTCAAATATT |
| | | GGTGGAATCTCCTACAATATTGGAGTCAGGAGCTAAAGAATAGTCT |
| | | AGA |
| 49 | Elongation | CCGGTGCCTAGAGAAGGTGGCGCGGGGTAAACTGGGAAAGTGATGT |
| | Factor-1 alpha | CGTGTACTGGCTCCGCCTTTTTCCCGAGGGTGGGGGAGAACCGTATA |
| | (EF1-alpha) | TAAGTGCAGTAGTCGCCGTGAACGTTCTTTTTCGCAACGGGTTTGCC |
| | promoter | GCCAGAACACAGGTAAGTGCCGTGTGTGGTTCCCGCGGGCCTGGCC |
| | | TCTTTACGGGTTATGGCCCTTGCGTGCCTTGAATTACTTCCACGCCCC |
| | | TGGCTGCAGTACGTGATTCTTGATCCCGAGCTTCGGGTTGGAAGTGG |
| | | GTGGGAGAGTTCGAGGCCTTGCGCTTAAGGAGCCCCTTCGCCTCGTG |
| | | CTTGAGTTGAGGCCTGGCCTGGGCGCTGGGGCCGCCGCGTGCGAAT |
| | | CTGGTGGCACCTTCGCGCCTGTCTCGCTGCTTTCGATAAGTCTCTAGC |
| | | CATTTAAAATTTTTGATGACCTGCTGCGACGCTTTTTTTCTGGCAAGA |
| | | TAGTCTTGTAAATGCGGGCCAAGATCTGCACACTGGTATTTCGGTTT |
| | | TTGGGGCCGCGGGCGGCGACGGGGCCCGTGCGTCCCAGCGCACATG |
| | | TTCGGCGAGGCGGGGCCTGCGAGCGCGGCCACCGAGAATCGGACGG |
| | | GGGTAGTCTCAAGCTGGCCGGCCTGCTCTGGTGCCTGGCCTCGCGCC |
| | | GCCGTGTATCGCCCCGCCCTGGGCGGCAAGGCTGGCCCGGTCGGCA |
| | | CCAGTTGCGTGAGCGGAAAGATGGCCGCTTCCCGGCCCTGCTGCAG |
| | | GGAGCTCAAAATGGAGGACGCGGCGCTCGGGAGAGCGGGCGGGTG |
| | | AGTCACCCACACAAAGGAAAAGGGCCTTTCCGTCCTCAGCCGTCGCT |
| | | TCATGTGACTCCACGGAGTACCGGGCGCCGTCCAGGCACCTCGATTA |
| | | GTTCTCGAGCTTTTGGAGTACGTCGTCTTTAGGTTGGGGGGAGGGGT |
| | | TTTATGCGATGGAGTTTCCCCACACTGAGTGGGTGGAGACTGAAGTT |
| | | AGGCCAGCTTGGCACTTGATGTAATTCTCCTTGGAATTTGCCCTTTTT |
| | | GAGTTTGGATCTTGGTTCATTCTCAAGCCTCAGACAGTGGTTCAAAG |
| | | TTTTTTTCTTCCATTTCAGGTGTCGTGA |
| 50 | Promoter; | GGGGTTGGGGTTGCGCCTTTTCCAAGGCAGCCCTGGGTTTGCGCAGG |
| | PGK | GACGCGGCTGCTCTGGGCGTGGTTCCGGGAAACGCAGCGGCGCCGA |
| | | CCCTGGGTCTCGCACATTCTTCACGTCCGTTCGCAGCGTCACCCGGA |
| | | TCTTCGCCGCTACCCTTGTGGGCCCCCCGGCGACGCTTCCTGCTCCG |
| | | CCCCTAAGTCGGGAAGGTTCCTTGCGGTTCGCGGCGTGCCGGACGTG |
| | | ACAAACGGAAGCCGCACGTCTCACTAGTACCCTCGCAGACGGACAG |
| | | CGCCAGGGAGCAATGGCAGCGCGCCGACCGCGATGGGCTGTGGCCA |
| | | ATAGCGGCTGCTCAGCAGGGCGCGCCGAGAGCAGCGGCCGGGAAG |
| | | GGGCGGTGCGGGAGGCGGGGTGTGGGGCGGTAGTGTGGGCCCTGTT |
| | | CCTGCCCGCGCGGTGTTCCGCATTCTGCAAGCCTCCGGAGCGCACGT |
| | | CGGCAGTCGGCTCCCTCGTTGACCGAATCACCGACCTCTCTCCCCAG |
| 51 | Promoter; | GCGCCGGGTTTTGGCGCCTCCCGCGGGCGCCCCCCTCCTCACGGCGA |
| | UbC | GCGCTGCCACGTCAGACGAAGGGCGCAGGAGCGTTCCTGATCCTTC |
| | | CGCCCGGACGCTCAGGACAGCGGCCCGCTGCTCATAAGACTCGGCC |
| | | TTAGAACCCCAGTATCAGCAGAAGGACATTTTAGGACGGGACTTGG |
| | | GTGACTCTAGGGCACTGGTTTTCTTTCCAGAGAGCGGAACAGGCGA |
| | | GGAAAAGTAGTCCCTTCTCGGCGATTCTGCGGAGGGATCTCCGTGGG |
| | | GCGGTGAACGCCGATGATTATATAAGGACGCGCCGGGTGTGGCACA |
| | | GCTAGTTCCGTCGCAGCCGGGATTTGGGTCGCGGTTCTTGTTTGTGG |
| | | ATCGCTGTGATCGTCACTTGGTGAGTTGCGGGCTGCTGGGCTGGCCG |
| | | GGGCTTTCGTGGCCGCCGGGCCGCTCGGTGGGACGGAAGCGTGTGG |
| | | AGAGACCGCCAAGGGCTGTAGTCTGGGTCCGCGAGCAAGGTTGCCC |
| | | TGAACTGGGGGTTGGGGGGAGCGCACAAAATGGCGGCTGTTCCCGA |
| | | GTCTTGAATGGAAGACGCTTGTAAGGCGGGCTGTGAGGTCGTTGAA |
| | | ACAAGGTGGGGGGCATGGTGGGCGGCAAGAACCCAAGGTCTTGAGG |
| | | CCTTCGCTAATGCGGGAAAGCTCTTATTCGGGTGAGATGGGCTGGGG |
| | | CACCATCTGGGGACCCTGACGTGAAGTTTGTCACTGACTGGAGAACT |
| | | CGGGTTTGTCGTCTGGTTGCGGGGGCGGCAGTTATGCGGTGCCGTTG |
| | | GGCAGTGCACCCGTACCTTTGGGAGCGCGCGCCTCGTCGTGTCGTGA |
| | | CGTCACCCGTTCTGTTGGCTTATAATGCAGGGTGGGGCCACCTGCCG |
| | | GTAGGTGTGCGGTAGGCTTTTCTCCGTCGCAGGACGCAGGGTTCGGG |
| | | CCTAGGGTAGGCTCTCCTGAATCGACAGGCGCCGGACCTCTGGTGA |
| | | GGGGAGGGATAAGTGAGGCGTCAGTTTCTTTGGTCGGTTTTATGTAC |
| | | CTATCTTCTTAAGTAGCTGAAGCTCCGGTTTTGAACTATGCGCTCGG |
| | | GGTTGGCGAGTGTGTTTTGTGAAGTTTTTTAGGCACCTTTTGAAATGT |
| | | AATCATTTGGGTCAATATGTAATTTTCAGTGTTAGACTAGTAAA |
| 52 | Poly A; SV40 | GTTTATTGCAGCTTATAATGGTTACAAATAAAGCAATAGCATCACAA |
| | | ATTTCACAAATAAAGCATTTTTTTCACTGCATTCTAGTTGTGGTTTGT |
| | | CCAAACTCATCAATGTATCTTATCA |
| 53 | Poly A; bGH | GACTGTGCCTTCTAGTTGCCAGCCATCTGTTGTTTGCCCCTCCCCCGT |
| | | GCCTTCCTTGACCCTGGAAGGTGCCACTCCCACTGTCCTTTCCTAATA |
| | | AAATGAGGAAATTGCATCGCATTGTCTGAGTAGGTGTCATTCTATTC |
| | | TGGGGGGTGGGGTGGGGCAGGACAGCAAGGGGGAGGATTGGGAAG |
| | | ACAATAGCAGGCATGCTGGGGATGCGGTGGGCTCTATGG |
| 54 | Envelope; | ATGAAACTCCCAACAGGAATGGTCATTTTATGTAGCCTAATAATAGT |
| | RD114 | TCGGGCAGGGTTTGACGACCCCCGCAAGGCTATCGCATTAGTACAA |
| | | AAACAACATGGTAAACCATGCGAATGCAGCGGAGGGCAGGTATCCG |
| | | AGGCCCCACCGAACTCCATCCAACAGGTAACTTGCCCAGGCAAGAC |
| | | GGCCTACTTAATGACCAACCAAAAATGGAAATGCAGAGTCACTCCA |
| | | AAAAATCTCACCCCTAGCGGGGGAGAACTCCAGAACTGCCCCTGTA |
| | | ACACTTTCCAGGACTCGATGCACAGTTCTTGTTATACTGAATACCGG |
| | | CAATGCAGGGCGAATAATAAGACATACTACACGGCCACCTTGCTTA |
| | | AAATACGGTCTGGGAGCCTCAACGAGGTACAGATATTACAAAACCC |
| | | CAATCAGCTCCTACAGTCCCCTTGTAGGGGCTCTATAAATCAGCCCG |
| | | TTTGCTGGAGTGCCACAGCCCCCATCCATATCTCCGATGGTGGAGGA |
| | | CCCCTCGATACTAAGAGAGTGTGGACAGTCCAAAAAAGGCTAGAAC |
| | | AAATTCATAAGGCTATGCATCCTGAACTTCAATACCACCCCTTAGCC |
| | | CTGCCCAAAGTCAGAGATGACCTTAGCCTTGATGCACGGACTTTTGA |
| | | TATCCTGAATACCACTTTTAGGTTACTCCAGATGTCCAATTTTAGCCT |
| | | TGCCCAAGATTGTTGGCTCTGTTTAAAACTAGGTACCCCTACCCCTC |
| | | TTGCGATACCCACTCCCTCTTTAACCTACTCCCTAGCAGACTCCCTAG |
| | | CGAATGCCTCCTGTCAGATTATACCTCCCCTCTTGGTTCAACCGATG |
| | | CAGTTCTCCAACTCGTCCTGTTTATCTTCCCCTTTCATTAACGATACG |
| | | GAACAAATAGACTTAGGTGCAGTCACCTTTACTAACTGCACCTCTGT |
| | | AGCCAATGTCAGTAGTCCTTTATGTGCCCTAAACGGGTCAGTCTTCC |
| | | TCTGTGGAAATAACATGGCATACACCTATTTACCCCAAAACTGGACA |
| | | GGACTTTGCGTCCAAGCCTCCCTCCTCCCCGACATTGACATCATCCC |
| | | GGGGGATGAGCCAGTCCCCATTCCTGCCATTGATCATTATATACATA |
| | | GACCTAAACGAGCTGTACAGTTCATCCCTTTACTAGCTGGACTGGGA |
| | | ATCACCGCAGCATTCACCACCGGAGCTACAGGCCTAGGTGTCTCCGT |
| | | CACCCAGTATACAAAATTATCCCATCAGTTAATATCTGATGTCCAAG |
| | | TCTTATCCGGTACCATACAAGATTTACAAGACCAGGTAGACTCGTTA |
| | | GCTGAAGTAGTTCTCCAAAATAGGAGGGGACTGGACCTACTAACGG |
| | | CAGAACAAGGAGGAATTTGTTTAGCCTTACAAGAAAAATGCTGTTTT |
| | | TATGCTAACAAGTCAGGAATTGTGAGAAACAAAATAAGAACCCTAC |
| | | AAGAAGAATTACAAAAACGCAGGGAAAGCCTGGCATCCAACCCTCT |
| | | CTGGACCGGGCTGCAGGGCTTTCTTCCGTACCTCCTACCTCTCCTGG |
| | | GACCCCTACTCACCCTCCTACTCATACTAACCATTGGGCCATGCGTT |
| | | TTCAATCGATTGGTCCAATTTGTTAAAGACAGGATCTCAGTGGTCCA |
| | | GGCTCTGGTTTTGACTCAGCAATATCACCAGCTAAAACCCATAGAGT |
| | | ACGAGCCATGA |
| 55 | Envelope; | ATGCTTCTCACCTCAAGCCCGCACCACCTTCGGCACCAGATGAGTCC |
| | GALV | TGGGAGCTGGAAAAGACTGATCATCCTCTTAAGCTGCGTATTCGGAG |
| | | ACGGCAAAACGAGTCTGCAGAATAAGAACCCCCACCAGCCTGTGAC |
| | | CCTCACCTGGCAGGTACTGTCCCAAACTGGGGACGTTGTCTGGGACA |
| | | AAAAGGCAGTCCAGCCCCTTTGGACTTGGTGGCCCTCTCTTACACCT |
| | | GATGTATGTGCCCTGGCGGCCGGTCTTGAGTCCTGGGATATCCCGGG |
| | | ATCCGATGTATCGTCCTCTAAAAGAGTTAGACCTCCTGATTCAGACT |
| | | ATACTGCCGCTTATAAGCAAATCACCTGGGGAGCCATAGGGTGCAG |
| | | CTACCCTCGGGCTAGGACCAGGATGGCAAATTCCCCCTTCTACGTGT |
| | | GTCCCCGAGCTGGCCGAACCCATTCAGAAGCTAGGAGGTGTGGGGG |
| | | GCTAGAATCCCTATACTGTAAAGAATGGAGTTGTGAGACCACGGGT |
| | | ACCGTTTATTGGCAACCCAAGTCCTCATGGGACCTCATAACTGTAAA |
| | | ATGGGACCAAAATGTGAAATGGGAGCAAAAATTTCAAAAGTGTGAA |
| | | CAAACCGGCTGGTGTAACCCCCTCAAGATAGACTTCACAGAAAAAG |
| | | GAAAACTCTCCAGAGATTGGATAACGGAAAAAACCTGGGAATTAAG |
| | | GTTCTATGTATATGGACACCCAGGCATACAGTTGACTATCCGCTTAG |
| | | AGGTCACTAACATGCCGGTTGTGGCAGTGGGCCCAGACCCTGTCCTT |
| | | GCGGAACAGGGACCTCCTAGCAAGCCCCTCACTCTCCCTCTCTCCCC |
| | | ACGGAAAGCGCCGCCCACCCCTCTACCCCCGGCGGCTAGTGAGCAA |
| | | ACCCCTGCGGTGCATGGAGAAACTGTTACCCTAAACTCTCCGCCTCC |
| | | CACCAGTGGCGACCGACTCTTTGGCCTTGTGCAGGGGGCCTTCCTAA |
| | | CCTTGAATGCTACCAACCCAGGGGCCACTAAGTCTTGCTGGCTCTGT |
| | | TTGGGCATGAGCCCCCCTTATTATGAAGGGATAGCCTCTTCAGGAGA |
| | | GGTCGCTTATACCTCCAACCATACCCGATGCCACTGGGGGGCCCAAG |
| | | GAAAGCTTACCCTCACTGAGGTCTCCGGACTCGGGTCATGCATAGGG |
| | | AAGGTGCCTCTTACCCATCAACATCTTTGCAACCAGACCTTACCCAT |
| | | CAATTCCTCTAAAAACCATCAGTATCTGCTCCCCTCAAACCATAGCT |
| | | GGTGGGCCTGCAGCACTGGCCTCACCCCCTGCCTCTCCACCTCAGTT |
| | | TTTAATCAGTCTAAAGACTTCTGTGTCCAGGTCCAGCTGATCCCCCG |
| | | CATCTATTACCATTCTGAAGAAACCTTGTTACAAGCCTATGACAAAT |
| | | CACCCCCCAGGTTTAAAAGAGAGCCTGCCTCACTTACCCTAGCTGTC |
| | | TTCCTGGGGTTAGGGATTGCGGCAGGTATAGGTACTGGCTCAACCGC |
| | | CCTAATTAAAGGGCCCATAGACCTCCAGCAAGGCCTAACCAGCCTC |
| | | CAAATCGCCATTGACGCTGACCTCCGGGCCCTTCAGGACTCAATCAG |
| | | CAAGCTAGAGGACTCACTGACTTCCCTATCTGAGGTAGTACTCCAAA |
| | | ATAGGAGAGGCCTTGACTTACTATTCCTTAAAGAAGGAGGCCTCTGC |
| | | GCGGCCCTAAAAGAAGAGTGCTGTTTTTATGTAGACCACTCAGGTGC |
| | | AGTACGAGACTCCATGAAAAAACTTAAAGAAAGACTAGATAAAAGA |
| | | CAGTTAGAGCGCCAGAAAAACCAAAACTGGTATGAAGGGTGGTTCA |
| | | ATAACTCCCCTTGGTTTACTACCCTACTATCAACCATCGCTGGGCCC |
| | | CTATTGCTCCTCCTTTTGTTACTCACTCTTGGGCCCTGCATCATCAAT |
| | | AAATTAATCCAATTCATCAATGATAGGATAAGTGCAGTCAAAATTTT |
| | | AGTCCTTAGACAGAAATATCAGACCCTAGATAACGAGGAAAACCTT |
| | | TAA |
| 56 | Envelope; | ATGGTTCCGCAGGTTCTTTTGTTTGTACTCCTTCTGGGTTTTTCGTTGT |
| | FUG | GTTTCGGGAAGTTCCCCATTTACACGATACCAGACGAACTTGGTCCC |
| | | TGGAGCCCTATTGACATACACCATCTCAGCTGTCCAAATAACCTGGT |
| | | TGTGGAGGATGAAGGATGTACCAACCTGTCCGAGTTCTCCTACATGG |
| | | AACTCAAAGTGGGATACATCTCAGCCATCAAAGTGAACGGGTTCAC |
| | | TTGCACAGGTGTTGTGACAGAGGCAGAGACCTACACCAACTTTGTTG |
| | | GTTATGTCACAACCACATTCAAGAGAAAGCATTTCCGCCCCACCCCA |
| | | GACGCATGTAGAGCCGCGTATAACTGGAAGATGGCCGGTGACCCCA |
| | | GATATGAAGAGTCCCTACACAATCCATACCCCGACTACCACTGGCTT |
| | | CGAACTGTAAGAACCACCAAAGAGTCCCTCATTATCATATCCCCAAG |
| | | TGTGACAGATTTGGACCCATATGACAAATCCCTTCACTCAAGGGTCT |
| | | TCCCTGGCGGAAAGTGCTCAGGAATAACGGTGTCCTCTACCTACTGC |
| | | TCAACTAACCATGATTACACCATTTGGATGCCCGAGAATCCGAGACC |
| | | AAGGACACCTTGTGACATTTTTACCAATAGCAGAGGGAAGAGAGCA |
| | | TCCAACGGGAACAAGACTTGCGGCTTTGTGGATGAAAGAGGCCTGT |
| | | ATAAGTCTCTAAAAGGAGCATGCAGGCTCAAGTTATGTGGAGTTCTT |
| | | GGACTTAGACTTATGGATGGAACATGGGTCGCGATGCAAACATCAG |
| | | ATGAGACCAAATGGTGCCCTCCAGATCAGTTGGTGAATTTGCACGAC |
| | | TTTCGCTCAGACGAGATCGAGCATCTCGTTGTGGAGGAGTTAGTTAA |
| | | GAAAAGAGAGGAATGTCTGGATGCATTAGAGTCCATCATGACCACC |
| | | AAGTCAGTAAGTTTCAGACGTCTCAGTCACCTGAGAAAACTTGTCCC |
| | | AGGGTTTGGAAAAGCATATACCATATTCAACAAAACCTTGATGGAG |
| | | GCTGATGCTCACTACAAGTCAGTCCGGACCTGGAATGAGATCATCCC |
| | | CTCAAAAGGGTGTTTGAAAGTTGGAGGAAGGTGCCATCCTCATGTG |
| | | AACGGGGTGTTTTTCAATGGTATAATATTAGGGCCTGACGACCATGT |
| | | CCTAATCCCAGAGATGCAATCATCCCTCCTCCAGCAACATATGGAGT |
| | | TGTTGGAATCTTCAGTTATCCCCCTGATGCACCCCCTGGCAGACCCT |
| | | TCTACAGTTTTCAAAGAAGGTGATGAGGCTGAGGATTTTGTTGAAGT |
| | | TCACCTCCCCGATGTGTACAAACAGATCTCAGGGGTTGACCTGGGTC |
| | | TCCCGAACTGGGGAAAGTATGTATTGATGACTGCAGGGGCCATGAT |
| | | TGGCCTGGTGTTGATATTTTCCCTAATGACATGGTGCAGAGTTGGTA |
| | | TCCATCTTTGCATTAAATTAAAGCACACCAAGAAAAGACAGATTTAT |
| | | ACAGACATAGAGATGAACCGACTTGGAAAGTAA |
| 57 | Envelope; | ATGGGTCAGATTGTGACAATGTTTGAGGCTCTGCCTCACATCATCGA |
| | LCMV | TGAGGTGATCAACATTGTCATTATTGTGCTTATCGTGATCACGGGTA |
| | | TCAAGGCTGTCTACAATTTTGCCACCTGTGGGATATTCGCATTGATC |
| | | AGTTTCCTACTTCTGGCTGGCAGGTCCTGTGGCATGTACGGTCTTAA |
| | | GGGACCCGACATTTACAAAGGAGTTTACCAATTTAAGTCAGTGGAG |
| | | TTTGATATGTCACATCTGAACCTGACCATGCCCAACGCATGTTCAGC |
| | | CAACAACTCCCACCATTACATCAGTATGGGGACTTCTGGACTAGAAT |
| | | TGACCTTCACCAATGATTCCATCATCAGTCACAACTTTTGCAATCTG |
| | | ACCTCTGCCTTCAACAAAAAGACCTTTGACCACACACTCATGAGTAT |
| | | AGTTTCGAGCCTACACCTCAGTATCAGAGGGAACTCCAACTATAAG |
| | | GCAGTATCCTGCGACTTCAACAATGGCATAACCATCCAATACAACTT |
| | | GACATTCTCAGATCGACAAAGTGCTCAGAGCCAGTGTAGAACCTTC |
| | | AGAGGTAGAGTCCTAGATATGTTTAGAACTGCCTTCGGGGGGAAAT |
| | | ACATGAGGAGTGGCTGGGGCTGGACAGGCTCAGATGGCAAGACCAC |
| | | CTGGTGTAGCCAGACGAGTTACCAATACCTGATTATACAAAATAGA |
| | | ACCTGGGAAAACCACTGCACATATGCAGGTCCTTTTGGGATGTCCAG |
| | | GATTCTCCTTTCCCAAGAGAAGACTAAGTTCTTCACTAGGAGACTAG |
| | | CGGGCACATTCACCTGGACTTTGTCAGACTCTTCAGGGGTGGAGAAT |
| | | CCAGGTGGTTATTGCCTGACCAAATGGATGATTCTTGCTGCAGAGCT |
| | | TAAGTGTTTCGGGAACACAGCAGTTGCGAAATGCAATGTAAATCAT |
| | | GATGCCGAATTCTGTGACATGCTGCGACTAATTGACTACAACAAGGC |
| | | TGCTTTGAGTAAGTTCAAAGAGGACGTAGAATCTGCCTTGCACTTAT |
| | | TCAAAACAACAGTGAATTCTTTGATTTCAGATCAACTACTGATGAGG |
| | | AACCACTTGAGAGATCTGATGGGGGTGCCATATTGCAATTACTCAAA |
| | | GTTTTGGTACCTAGAACATGCAAAGACCGGCGAAACTAGTGTCCCC |
| | | AAGTGCTGGCTTGTCACCAATGGTTCTTACTTAAATGAGACCCACTT |
| | | CAGTGATCAAATCGAACAGGAAGCCGATAACATGATTACAGAGATG |
| | | TTGAGGAAGGATTACATAAAGAGGCAGGGGAGTACCCCCCTAGCAT |
| | | TGATGGACCTTCTGATGTTTTCCACATCTGCATATCTAGTCAGCATCT |
| | | TCCTGCACCTTGTCAAAATACCAACACACAGGCACATAAAAGGTGG |
| | | CTCATGTCCAAAGCCACACCGATTAACCAACAAAGGAATTTGTAGTT |
| | | GTGGTGCATTTAAGGTGCCTGGTGTAAAAACCGTCTGGAAAAGACG |
| | | CTGA |
| 58 | Envelope; | ATGAACACTCAAATCCTGGTTTTCGCCCTTGTGGCAGTCATCCCCAC |
| | FPV | AAATGCAGACAAAATTTGTCTTGGACATCATGCTGTATCAAATGGCA |
| | | CCAAAGTAAACACACTCACTGAGAGAGGAGTAGAAGTTGTCAATGC |
| | | AACGGAAACAGTGGAGCGGACAAACATCCCCAAAATTTGCTCAAAA |
| | | GGGAAAAGAACCACTGATCTTGGCCAATGCGGACTGTTAGGGACCA |
| | | TTACCGGACCACCTCAATGCGACCAATTTCTAGAATTTTCAGCTGAT |
| | | CTAATAATCGAGAGACGAGAAGGAAATGATGTTTGTTACCCGGGGA |
| | | AGTTTGTTAATGAAGAGGCATTGCGACAAATCCTCAGAGGATCAGG |
| | | TGGGATTGACAAAGAAACAATGGGATTCACATATAGTGGAATAAGG |
| | | ACCAACGGAACAACTAGTGCATGTAGAAGATCAGGGTCTTCATTCT |
| | | ATGCAGAAATGGAGTGGCTCCTGTCAAATACAGACAATGCTGCTTTC |
| | | CCACAAATGACAAAATCATACAAAAACACAAGGAGAGAATCAGCTC |
| | | TGATAGTCTGGGGAATCCACCATTCAGGATCAACCACCGAACAGAC |
| | | CAAACTATATGGGAGTGGAAATAAACTGATAACAGTCGGGAGTTCC |
| | | AAATATCATCAATCTTTTGTGCCGAGTCCAGGAACACGACCGCAGAT |
| | | AAATGGCCAGTCCGGACGGATTGATTTTCATTGGTTGATCTTGGATC |
| | | CCAATGATACAGTTACTTTTAGTTTCAATGGGGCTTTCATAGCTCCA |
| | | AATCGTGCCAGCTTCTTGAGGGGAAAGTCCATGGGGATCCAGAGCG |
| | | ATGTGCAGGTTGATGCCAATTGCGAAGGGGAATGCTACCACAGTGG |
| | | AGGGACTATAACAAGCAGATTGCCTTTTCAAAACATCAATAGCAGA |
| | | GCAGTTGGCAAATGCCCAAGATATGTAAAACAGGAAAGTTTATTAT |
| | | TGGCAACTGGGATGAAGAACGTTCCCGAACCTTCCAAAAAAAGGAA |
| | | AAAAAGAGGCCTGTTTGGCGCTATAGCAGGGTTTATTGAAAATGGTT |
| | | GGGAAGGTCTGGTCGACGGGTGGTACGGTTTCAGGCATCAGAATGC |
| | | ACAAGGAGAAGGAACTGCAGCAGACTACAAAAGCACCCAATCGGC |
| | | AATTGATCAGATAACCGGAAAGTTAAATAGACTCATTGAGAAAACC |
| | | AACCAGCAATTTGAGCTAATAGATAATGAATTCACTGAGGTGGAAA |
| | | AGCAGATTGGCAATTTAATTAACTGGACCAAAGACTCCATCACAGA |
| | | AGTATGGTCTTACAATGCTGAACTTCTTGTGGCAATGGAAAACCAGC |
| | | ACACTATTGATTTGGCTGATTCAGAGATGAACAAGCTGTATGAGCGA |
| | | GTGAGGAAACAATTAAGGGAAAATGCTGAAGAGGATGGCACTGGTT |
| | | GCTTTGAAATTTTTCATAAATGTGACGATGATTGTATGGCTAGTATA |
| | | AGGAACAATACTTATGATCACAGCAAATACAGAGAAGAAGCGATGC |
| | | AAAATAGAATACAAATTGACCCAGTCAAATTGAGTAGTGGCTACAA |
| | | AGATGTGATACTTTGGTTTAGCTTCGGGGCATCATGCTTTTTGCTTCT |
| | | TGCCATTGCAATGGGCCTTGTTTTCATATGTGTGAAGAACGGAAACA |
| | | TGCGGTGCACTATTTGTATATAA |
| 59 | Envelope; | AGTGTAACAGAGCACTTTAATGTGTATAAGGCTACTAGACCATACCT |
| | RRV | AGCACATTGCGCCGATTGCGGGGACGGGTACTTCTGCTATAGCCCAG |
| | | TTGCTATCGAGGAGATCCGAGATGAGGCGTCTGATGGCATGCTTAA |
| | | GATCCAAGTCTCCGCCCAAATAGGTCTGGACAAGGCAGGCACCCAC |
| | | GCCCACACGAAGCTCCGATATATGGCTGGTCATGATGTTCAGGAATC |
| | | TAAGAGAGATTCCTTGAGGGTGTACACGTCCGCAGCGTGCTCCATAC |
| | | ATGGGACGATGGGACACTTCATCGTCGCACACTGTCCACCAGGCGA |
| | | CTACCTCAAGGTTTCGTTCGAGGACGCAGATTCGCACGTGAAGGCAT |
| | | GTAAGGTCCAATACAAGCACAATCCATTGCCGGTGGGTAGAGAGAA |
| | | GTTCGTGGTTAGACCACACTTTGGCGTAGAGCTGCCATGCACCTCAT |
| | | ACCAGCTGACAACGGCTCCCACCGACGAGGAGATTGACATGCATAC |
| | | ACCGCCAGATATACCGGATCGCACCCTGCTATCACAGACGGCGGGC |
| | | AACGTCAAAATAACAGCAGGCGGCAGGACTATCAGGTACAACTGTA |
| | | CCTGCGGCCGTGACAACGTAGGCACTACCAGTACTGACAAGACCAT |
| | | CAACACATGCAAGATTGACCAATGCCATGCTGCCGTCACCAGCCAT |
| | | GACAAATGGCAATTTACCTCTCCATTTGTTCCCAGGGCTGATCAGAC |
| | | AGCTAGGAAAGGCAAGGTACACGTTCCGTTCCCTCTGACTAACGTCA |
| | | CCTGCCGAGTGCCGTTGGCTCGAGCGCCGGATGCCACCTATGGTAAG |
| | | AAGGAGGTGACCCTGAGATTACACCCAGATCATCCGACGCTCTTCTC |
| | | CTATAGGAGTTTAGGAGCCGAACCGCACCCGTACGAGGAATGGGTT |
| | | GACAAGTTCTCTGAGCGCATCATCCCAGTGACGGAAGAAGGGATTG |
| | | AGTACCAGTGGGGCAACAACCCGCCGGTCTGCCTGTGGGCGCAACT |
| | | GACGACCGAGGGCAAACCCCATGGCTGGCCACATGAAATCATTCAG |
| | | TACTATTATGGACTATACCCCGCCGCCACTATTGCCGCAGTATCCGG |
| | | GGCGAGTCTGATGGCCCTCCTAACTCTGGCGGCCACATGCTGCATGC |
| | | TGGCCACCGCGAGGAGAAAGTGCCTAACACCGTACGCCCTGACGCC |
| | | AGGAGCGGTGGTACCGTTGACACTGGGGCTGCTTTGCTGCGCACCG |
| | | AGGGCGAATGCA |
| 60 | Envelope; | ATGGAAGGTCCAGCGTTCTCAAAACCCCTTAAAGATAAGATTAACC |
| | MLV 10A1 | CGTGGAAGTCCTTAATGGTCATGGGGGTCTATTTAAGAGTAGGGATG |
| | | GCAGAGAGCCCCCATCAGGTCTTTAATGTAACCTGGAGAGTCACCA |
| | | ACCTGATGACTGGGCGTACCGCCAATGCCACCTCCCTTTTAGGAACT |
| | | GTACAAGATGCCTTCCCAAGATTATATTTTGATCTATGTGATCTGGT |
| | | CGGAGAAGAGTGGGACCCTTCAGACCAGGAACCATATGTCGGGTAT |
| | | GGCTGCAAATACCCCGGAGGGAGAAAGCGGACCCGGACTTTTGACT |
| | | TTTACGTGTGCCCTGGGCATACCGTAAAATCGGGGTGTGGGGGGCC |
| | | AAGAGAGGGCTACTGTGGTGAATGGGGTTGTGAAACCACCGGACAG |
| | | GCTTACTGGAAGCCCACATCATCATGGGACCTAATCTCCCTTAAGCG |
| | | CGGTAACACCCCCTGGGACACGGGATGCTCCAAAATGGCTTGTGGC |
| | | CCCTGCTACGACCTCTCCAAAGTATCCAATTCCTTCCAAGGGGCTAC |
| | | TCGAGGGGGCAGATGCAACCCTCTAGTCCTAGAATTCACTGATGCA |
| | | GGAAAAAAGGCTAATTGGGACGGGCCCAAATCGTGGGGACTGAGAC |
| | | TGTACCGGACAGGAACAGATCCTATTACCATGTTCTCCCTGACCCGC |
| | | CAGGTCCTCAATATAGGGCCCCGCATCCCCATTGGGCCTAATCCCGT |
| | | GATCACTGGTCAACTACCCCCCTCCCGACCCGTGCAGATCAGGCTCC |
| | | CCAGGCCTCCTCAGCCTCCTCCTACAGGCGCAGCCTCTATAGTCCCT |
| | | GAGACTGCCCCACCTTCTCAACAACCTGGGACGGGAGACAGGCTGC |
| | | TAAACCTGGTAGAAGGAGCCTATCAGGCGCTTAACCTCACCAATCCC |
| | | GACAAGACCCAAGAATGTTGGCTGTGCTTAGTGTCGGGACCTCCTTA |
| | | TTACGAAGGAGTAGCGGTCGTGGGCACTTATACCAATCATTCTACCG |
| | | CCCCGGCCAGCTGTACGGCCACTTCCCAACATAAGCTTACCCTATCT |
| | | GAAGTGACAGGACAGGGCCTATGCATGGGAGCACTACCTAAAACTC |
| | | ACCAGGCCTTATGTAACACCACCCAAAGTGCCGGCTCAGGATCCTAC |
| | | TACCTTGCAGCACCCGCTGGAACAATGTGGGCTTGTAGCACTGGATT |
| | | GACTCCCTGCTTGTCCACCACGATGCTCAATCTAACCACAGACTATT |
| | | GTGTATTAGTTGAGCTCTGGCCCAGAATAATTTACCACTCCCCCGAT |
| | | TATATGTATGGTCAGCTTGAACAGCGTACCAAATATAAGAGGGAGC |
| | | CAGTATCGTTGACCCTGGCCCTTCTGCTAGGAGGATTAACCATGGGA |
| | | GGGATTGCAGCTGGAATAGGGACGGGGACCACTGCCCTAATCAAAA |
| | | CCCAGCAGTTTGAGCAGCTTCACGCCGCTATCCAGACAGACCTCAAC |
| | | GAAGTCGAAAAATCAATTACCAACCTAGAAAAGTCACTGACCTCGT |
| | | TGTCTGAAGTAGTCCTACAGAACCGAAGAGGCCTAGATTTGCTCTTC |
| | | CTAAAAGAGGGAGGTCTCTGCGCAGCCCTAAAAGAAGAATGTTGTT |
| | | TTTATGCAGACCACACGGGACTAGTGAGAGACAGCATGGCCAAACT |
| | | AAGGGAAAGGCTTAATCAGAGACAAAAACTATTTGAGTCAGGCCAA |
| | | GGTTGGTTCGAAGGGCAGTTTAATAGATCCCCCTGGTTTACCACCTT |
| | | AATCTCCACCATCATGGGACCTCTAATAGTACTCTTACTGATCTTACT |
| | | CTTTGGACCCTGCATTCTCAATCGATTGGTCCAATTTGTTAAAGACA |
| | | GGATCTCAGTGGTCCAGGCTCTGGTTTTGACTCAACAATATCACCAG |
| | | CTAAAACCTATAGAGTACGAGCCATGA |
| 61 | Envelope; | ATGGGTGTTACAGGAATATTGCAGTTACCTCGTGATCGATTCAAGAG |
| | Ebola | GACATCATTCTTTCTTTGGGTAATTATCCTTTTCCAAAGAACATTTTC |
| | | CATCCCACTTGGAGTCATCCACAATAGCACATTACAGGTTAGTGATG |
| | | TCGACAAACTGGTTTGCCGTGACAAACTGTCATCCACAAATCAATTG |
| | | AGATCAGTTGGACTGAATCTCGAAGGGAATGGAGTGGCAACTGACG |
| | | TGCCATCTGCAACTAAAAGATGGGGCTTCAGGTCCGGTGTCCCACCA |
| | | AAGGTGGTCAATTATGAAGCTGGTGAATGGGCTGAAAACTGCTACA |
| | | ATCTTGAAATCAAAAAACCTGACGGGAGTGAGTGTCTACCAGCAGC |
| | | GCCAGACGGGATTCGGGGCTTCCCCCGGTGCCGGTATGTGCACAAA |
| | | GTATCAGGAACGGGACCGTGTGCCGGAGACTTTGCCTTCCACAAAG |
| | | AGGGTGCTTTCTTCCTGTATGACCGACTTGCTTCCACAGTTATCTACC |
| | | GAGGAACGACTTTCGCTGAAGGTGTCGTTGCATTTCTGATACTGCCC |
| | | CAAGCTAAGAAGGACTTCTTCAGCTCACACCCCTTGAGAGAGCCGG |
| | | TCAATGCAACGGAGGACCCGTCTAGTGGCTACTATTCTACCACAATT |
| | | AGATATCAAGCTACCGGTTTTGGAACCAATGAGACAGAGTATTTGTT |
| | | CGAGGTTGACAATTTGACCTACGTCCAACTTGAATCAAGATTCACAC |
| | | CACAGTTTCTGCTCCAGCTGAATGAGACAATATATACAAGTGGGAA |
| | | AAGGAGCAATACCACGGGAAAACTAATTTGGAAGGTCAACCCCGAA |
| | | ATTGATACAACAATCGGGGAGTGGGCCTTCTGGGAAACTAAAAAAA |
| | | CCTCACTAGAAAAATTCGCAGTGAAGAGTTGTCTTTCACAGCTGTAT |
| | | CAAACAGAGCCAAAAACATCAGTGGTCAGAGTCCGGCGCGAACTTC |
| | | TTCCGACCCAGGGACCAACACAACAACTGAAGACCACAAAATCATG |
| | | GCTTCAGAAAATTCCTCTGCAATGGTTCAAGTGCACAGTCAAGGAA |
| | | GGGAAGCTGCAGTGTCGCATCTGACAACCCTTGCCACAATCTCCACG |
| | | AGTCCTCAACCCCCCACAACCAAACCAGGTCCGGACAACAGCACCC |
| | | ACAATACACCCGTGTATAAACTTGACATCTCTGAGGCAACTCAAGTT |
| | | GAACAACATCACCGCAGAACAGACAACGACAGCACAGCCTCCGACA |
| | | CTCCCCCCGCCACGACCGCAGCCGGACCCCTAAAAGCAGAGAACAC |
| | | CAACACGAGCAAGGGTACCGACCTCCTGGACCCCGCCACCACAACA |
| | | AGTCCCCAAAACCACAGCGAGACCGCTGGCAACAACAACACTCATC |
| | | ACCAAGATACCGGAGAAGAGAGTGCCAGCAGCGGGAAGCTAGGCTT |
| | | AATTACCAATACTATTGCTGGAGTCGCAGGACTGATCACAGGCGGG |
| | | AGGAGAGCTCGAAGAGAAGCAATTGTCAATGCTCAACCCAAATGCA |
| | | ACCCTAATTTACATTACTGGACTACTCAGGATGAAGGTGCTGCAATC |
| | | GGACTGGCCTGGATACCATATTTCGGGCCAGCAGCCGAGGGAATTT |
| | | ACATAGAGGGGCTGATGCACAATCAAGATGGTTTAATCTGTGGGTT |
| | | GAGACAGCTGGCCAACGAGACGACTCAAGCTCTTCAACTGTTCCTG |
| | | AGAGCCACAACCGAGCTACGCACCTTTTCAATCCTCAACCGTAAGGC |
| | | AATTGATTTCTTGCTGCAGCGATGGGGCGGCACATGCCACATTTTGG |
| | | GACCGGACTGCTGTATCGAACCACATGATTGGACCAAGAACATAAC |
| | | AGACAAAATTGATCAGATTATTCATGATTTTGTTGATAAAACCCTTC |
| | | CGGACCAGGGGGACAATGACAATTGGTGGACAGGATGGAGACAAT |
| | | GGATACCGGCAGGTATTGGAGTTACAGGCGTTATAATTGCAGTTATC |
| | | GCTTTATTCTGTATATGCAAATTTGTCTTTTAG |
| 62 | Thyroxin | CTTTCTCTTTTGTTTTACATGAAGGGTCTGGCAGCCAAAGCAATCAC |
| | binding | TCAAAGTTCAAACCTTATCATTTTTTGCTTTGTTCCTCTTGGCCTTGG |
| | globulin | TTTTGTACATCAGCTTTGAAAATACCATCCCAGGGTTAATGCTGGGG |
| | promoter | TTAATTTATAACTAAGAGTGCTCTAGTTTTGCAATACAGGACATGCT |
| | (TBG) | ATAAAAATGGAAAGATGTTGCTTTCTGAG |
| 63 | DNA | GCGAGAACTTGTGCCTCCCCGTGTTCCTGCTCTTTGTCCCTCTGTCCT |
| | fragment | ACTTAGACTAATATTTGCCTTGGGTACTGCAAACAGGAAATGGGGG |
| | containing | AGGGACAGGAGTAGGGCGGAGGGTAGCCCGGGGATCTTGCTACCAG |
| | prothrombin | TGGAACAGCCACTAAGGATTCTGCAGTGAGAGCAGAGGGCCAGCTA |
| | enhancer and | AGTGGTACTCTCCCAGAGACTGTCTGACTCACGCCACCCCCTCCACC |
| | human alpha-1 | TTGGACACAGGACGCTGTGGTTTCTGAGCCAGGTACAATGACTCCTT |
| | anti-trypsin | TCGGTAAGTGCAGTGGAAGCTGTACACTGCCCAGGCAAAGCGTCCG |
| | promoter | GGCAGCGTAGGCGGGCGACTCAGATCCCAGCCAGTGGACTTAGCCC |
| | | CTGTTTGCTCCTCCGATAACTGGGGTGACCTTGGTTAATATTCACCA |
| | | GCAGCCTCCCCCGTTGCCCCTCTGGATCCACTGCTTAAATACGGACG |
| | | AGGACAGGGCCCTGTCTCCTCAGCTTCAGGCACCACCACTGACCTGG |
| | | GACAGTGAAT |
| 64 | DNA | GTTAATCATTAACGTTAATCATTAACGTTAATCATTAACGTTAATCA |
| | fragment | TTAACGTTAATCATTAACATCGATGCGAGAACTTGTGCCTCCCCGTG |
| | containing | TTCCTGCTCTTTGTCCCTCTGTCCTACTTAGACTAATATTTGCCTTGG |
| | prothrombin | GTACTGCAAACAGGAAATGGGGGAGGGACAGGAGTAGGGCGGAGG |
| | enhancer, | GTAGGATTCTGCAGTGAGAGCAGAGGGCCAGCTAAGTGGTACTCTC |
| | human alpha-1 | CCAGAGACTGTCTGACTCACGCCACCCCCTCCACCTTGGACACAGGA |
| | anti-trypsin | CGCTGTGGTTTCTGAGCCAGGTACAATGACTCCTTTCGGTAAGTGCA |
| | promoter, and | GTGGAAGCTGTACACTGCCCAGGCAAAGCGTCCGGGCAGCGTAGGC |
| | five HNF1 | GGGCGACTCAGATCCCAGCCAGTGGACTTAGCCCCTGTTTGCTCCTC |
| | binding sites | CGATAACTGGGGTGACCTTGGTTAATATTCACCAGCAGCCTCCCCCG |
| | | TTGCCCCTCTGGATCCACTGCTTAAATACGGACGAGGACAGGGCCCT |
| | | GTCTCCTCAGCTTCAGGCACCACCACTGACCTGGGACAGTGAAT |
| 65 | DNA | GTTAATCATTAACGCTTGTACTTTGGTACAGTTAATCATTAACGCTTG |
| | fragment | TACTTTGGTACAGTTAATCATTAACGCTTGTACTTTGGTACAATCGAT |
| | containing | GCGAGAACTTGTGCCTCCCCGTGTTCCTGCTCTTTGTCCCTCTGTCCT |
| | prothrombin | ACTTAGACTAATATTTGCCTTGGGTACTGCAAACAGGAAATGGGGG |
| | enhancer, | AGGGACAGGAGTAGGGCGGAGGGTAGCCCGGGGATTCTGCAGTGA |
| | human alpha- | GAGCAGAGGGCCAGCTAAGTGGTACTCTCCCAGAGACTGTCTGACT |
| | 1, anti-trypsin | CACGCCACCCCCTCCACCTTGGACACAGGACGCTGTGGTTTCTGAGC |
| | promoter, and | CAGGTACAATGACTCCTTTCGGTAAGTGCAGTGGAAGCTGTACACTG |
| | three | CCCAGGCAAAGCGTCCGGGCAGCGTAGGCGGGCGACTCAGATCCCA |
| | HNF1/HNF4 | GCCAGTGGACTTAGCCCCTGTTTGCTCCTCCGATAACTGGGGTGACC |
| | binding sites | TTGGTTAATATTCACCAGCAGCCTCCCCCGTTGCCCCTCTGGATCCA |
| | | CTGCTTAAATACGGACGAGGACAGGGCCCTGTCTCCTCAGCTTCAGG |
| | | CACCACCACTGACCTGGGACAGTGAAT |
| 66 | Taqman Probe | TCGTGAAAGCTCATGGACAGTGGC |
| 67 | Taqman | AGATCTTGAGGCATGACATTGG |
| | Forward | |
| | Primer | |
| 68 | Taqman | GTCCAGCTCTTGAATGGTTCTT |
| | Reverse | |
| | Primer | |
| 69 | Actin Probe | AGCGGGAAATCGTGCGTGAC |
| 70 | Actin Forward | GGACCTGACTGACTACCTCAT |
| | Primer | |
| 71 | Actin Reverse | CGTAGCACAGCTTCTCCTTAAT |
| | Primer | |

1. A viral vector comprising a therapeutic cargo portion, wherein the therapeutic cargo portion comprises:
a PAH sequence or a variant thereof,
a promoter; and
a liver-specific enhancer,
wherein the PAH sequence or the variant thereof is operatively controlled by both the promoter and the liver-specific enhancer.

2. The viral vector of claim 1, wherein the liver-specific enhancer comprises a prothrombin enhancer.

3. The viral vector of claim 2, wherein the promoter comprises a liver-specific promoter.

4. The viral vector of claim 3, wherein the liver-specific promoter comprises a hAAT promoter.

5. The viral vector of claim 1, wherein the PAH sequence or the variant thereof is truncated.

6. The viral vector of claim 5, wherein the PAH sequence or the variant thereof is truncated at the 3′ untranslated region (UTR) of the PAH sequence or the variant thereof.

7. The viral vector of claim 1, wherein the therapeutic cargo portion further comprises a beta globin intron.

8. The viral vector of claim 2, wherein the therapeutic cargo portion further comprises at least one hepatocyte nuclear factor binding site.

9. The viral vector of claim 8, wherein the at least one hepatocyte nuclear factor binding site is disposed upstream of the prothrombin enhancer or downstream of the prothrombin enhancer.

10. (canceled)

11. The viral vector of claim 1, wherein the PAH sequence or the variant thereof comprises a sequence having at least 80% identity with SEQ ID NO: 1; SEQ ID NO: 2; SEQ ID NO: 3; or SEQ ID NO: 4.

12. (canceled)

13. The viral vector of claim 2, wherein the prothrombin enhancer comprises a sequence having at least 80% identity with SEQ ID NO: 5.

14. (canceled)

15. The viral vector of claim 4, wherein the hAAT promoter comprises a sequence having at least 80% identity with SEQ ID NO: 6.

16. The viral vector of claim 7, wherein the beta globin intron comprises a sequence having at least 80% identity with SEQ ID NO: 7 or SEQ ID NO: 8.

17. The viral vector of claim 8, wherein the at least one hepatocyte nuclear factor binding site comprises a sequence having at least 80% identity with any one of SEQ ID NO: 9; SEQ ID NO: 10; SEQ ID NO: 11; or SEQ ID NO: 12.

18. The viral vector of claim 1, wherein the therapeutic cargo portion further comprises at least one small RNA sequence that is capable of binding to at least one pre-determined complementary mRNA sequence.

19. The viral vector of claim 18, wherein the at least one pre-determined complementary mRNA sequence comprises a full-length UTR.

20. The viral vector of claim 19, wherein the at least one pre-determined complementary mRNA sequence comprises a PAH mRNA sequence.

21. The viral vector of claim 18, wherein the at least one small RNA sequence comprises a shRNA.

22. The viral vector of claim 18, wherein the at least one small RNA sequence is under the control of a first promoter and the PAH sequence or the variant thereof is under the control of a second promoter.

23. The viral vector of claim 22, wherein the first promoter comprises a H1 promoter and the second promoter comprises a liver-specific promoter.

24. (canceled)

25. The viral vector of claim 23, wherein the liver-specific promoter comprises a hAAT promoter.

26. The viral vector of claim 18, wherein the at least one small RNA sequence comprises a sequence having at least 80% identity with SEQ ID NO: 13 or SEQ ID NO: 14.

27. (canceled)

28. The viral vector of claim 1, wherein the viral vector is a lentiviral vector.

29. A lentiviral particle capable of infecting a target cell, the lentiviral particle comprising:
an envelope protein; and
the viral vector according to claim 1.

30. The lentiviral particle of claim 29, wherein the target cell is at least one of a hepatic cell, a muscle cell, an epithelial cell, an endothelial cell, a neural cell, a neuroendocrine cell, an endocrine cell, a lymphocyte, a myeloid cell, a cell present within a solid organ, or cell of a hematopoietic lineage, a hematopoietic stem cell, or a precursor hematopoietic stem cell.

31. A method of treating or preventing phenylketonuria in a subject, comprising administering to the subject a therapeutically effective amount of the lentiviral particle of claim 29.

32. (canceled)

33. The method of claim 31 further comprising diagnosing a PKU genotype in the subject that correlates with a PKU phenotype.

34. The method of claim 31, wherein the subject is in utero.

35. The method of claim 33, wherein the diagnosing occurs during prenatal screening of the subject.

36. The method of claim 33, wherein the diagnosing occurs in vitro.

37. The method of claim 31, wherein the therapeutically effective amount of the lentiviral particle comprises at least one of a single dose or a plurality of single doses of the lentiviral particle.

38. (canceled)
